SEQUENCE LISTING (21 sequences)

SEQ ID NO: 1 - DNA, 22 bp, Artificial Sequence
  Synthetic Construct
cctttcttctgtcgagatcagg

SEQ ID NO: 2 - DNA, 25 bp, Artificial Sequence
  Synthetic Construct
ccctgttgacttctctaatctcttc

SEQ ID NO: 3 - DNA, 20 bp, Artificial Sequence
  Synthetic Construct
cctgcagcccactgataaat

SEQ ID NO: 4 - DNA, 19 bp, Artificial Sequence
  Synthetic Construct
gggaccacaacctggattc

SEQ ID NO: 5 - DNA, 19 bp, Artificial Sequence
  Synthetic Construct
gacacagagtccggcattg

SEQ ID NO: 6 - DNA, 20 bp, Artificial Sequence
  Synthetic Construct
gcgagggatcctaactctca

SEQ ID NO: 7 - DNA, 19 bp, Artificial Sequence
  Synthetic Construct
caaggtgcgcgtatgctac

SEQ ID NO: 8 - DNA, 18 bp, Artificial Sequence
  Synthetic Construct
ggcttggagggaagttgg

SEQ ID NO: 9 - DNA, 19 bp, Artificial Sequence
  Synthetic Construct
cctccatgagaggcttcct

SEQ ID NO: 10 - DNA, 19 bp, Artificial Sequence
  Synthetic Construct
taggggccggtctaggact

SEQ ID NO: 11 - DNA, 18 bp, Artificial Sequence
  Synthetic Construct
ttctggttccgcatcagg

SEQ ID NO: 12 - DNA, 20 bp, Artificial Sequence
  Synthetic Construct
aaggccagagtaaccgagtg

SEQ ID NO: 13 - DNA, 20 bp, Artificial Sequence
  Synthetic Construct
gcttagctgggagtccacat

SEQ ID NO: 14 - DNA, 18 bp, Artificial Sequence
  Synthetic Construct
gcagccttgtcagcacac

SEQ ID NO: 15 - DNA, 18 bp, Artificial Sequence
  Synthetic Construct
atggctctggggacacac

SEQ ID NO: 16 - DNA, 21 bp, Artificial Sequence
  Synthetic Construct
tgtagaaggagtggtgccaga

SEQ ID NO: 17 - DNA, 22 bp, Artificial Sequence
  Synthetic Construct
gctcattgatgatccgcaacac

SEQ ID NO: 18 - DNA, 18 bp, Artificial Sequence
  Synthetic Construct
agcctgcctgaaatgcac

SEQ ID NO: 19 - DNA, 19 bp, Artificial Sequence
  Synthetic Construct
ctcactctggtggcaaagg

SEQ ID NO: 20 - DNA, 19 bp, Artificial Sequence
  Synthetic Construct
ccttcccacctctgagcat

SEQ ID NO: 21 - protein, 2555 aa, Artificial Sequence
  Synthetic Construct
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

(19) United States
(12) Patent Application Publication
(10) Pub. No.: US 2023/0103142 A1
(43) Pub. Date: Mar. 30, 2023

(54) SMALL MOLECULE REGULATORS OF NOTCH1 AND USES THEREOF

(71) Applicants: THE PENN STATE RESEARCH FOUNDATION, University Park, PA (US); UNIVERSITY OF LOUISVILLE RESEARCH FOUNDATION, INC., Louisville, KY (US)

(72) Inventors: Arun K. SHARMA, Hummelstown, PA (US); Ashish TYAGI, College Station, TX (US); Chendil DAMODARAN, College Station, TX (US); Shantu AMIN, Union City, NJ (US)

(21) Appl. No.: 17/799,795

(22) PCT Filed: Feb. 15, 2021
(86) PCT No.: PCT/US2021/018119

§ 371 Date: Aug. 15, 2022

Related U.S. Application Data
(60) Provisional application No. 62/976,880, filed on Feb. 14, 2020.

(51) Int. Cl. C07J 17/00; C07J 33/00; C07J 43/00; A61P 25/28

(52) U.S. Cl. CPC C07J 17/00; C07J 33/002; C07J 43/003; A61P 25/28

(57) ABSTRACT

Disclosed herein are compositions and methods relating to treating, preventing, reducing, and/or inhibiting cancers, infectious diseases, and/or neurological disorders.

### CROSS-REFERENCE TO RELATED APPLICATIONS

This application claims the benefit of U.S. Provisional Application No. 62/976,880, filed Feb. 14, 2020, which is expressly incorporated herein by reference in its entirety.

### STATEMENT REGARDING FEDERALLY SPONSORED RESEARCH OR DEVELOPMENT

This invention was made with Government Support under Grant NO: CA185972 awarded by the National Institutes of Health. The Government has certain rights in the invention.

### BACKGROUND

Cancer is the second leading cause of death in the United States, exceeded only by heart disease. In 2017, the latest year for which incidence data are available, in the United States, 1,701,315 new cases of cancer were reported, and 599,099 people died of cancer. For every 100,000 people, 438 new cancer cases were reported and 153 people died of cancer. Although great strides have been made in antibody engineering and cancer therapy, production cost is estimated at twice of that required for conventional drugs, which makes its use restrictive to large number of users. For example, the calculated per patient cost of treatment of colorectal cancer with CmAbs (bevacizumab, cetuximab and panitumumab) is US$30,400 in comparison to US$17,500 for the use of conventional chemotherapeutic drugs (for examples, oxaliplatin, irinotecan, fluorouracil and leucovorin). Moreover, while the introduction of small drug molecules provides cost savings of 80% to USA medical expense, no such benefit occurs with the biological substitutes (biosimilars) such as mAbs, where the savings amounts to 30% at best. What is needed are new small molecules for treating cancers.

### SUMMARY

Disclosed herein are compositions and methods for treating, preventing, reducing, and/or inhibiting cancers, infectious diseases, and/or neurological disorders.

The details of one or more embodiments of the invention are set forth in the accompanying drawings and the description below. Other features, objects, and advantages of the invention will be apparent from the description and drawings, and from the claims.

In one aspect, disclosed herein is a compound provided of Formula I, Formula II, or Formula III:

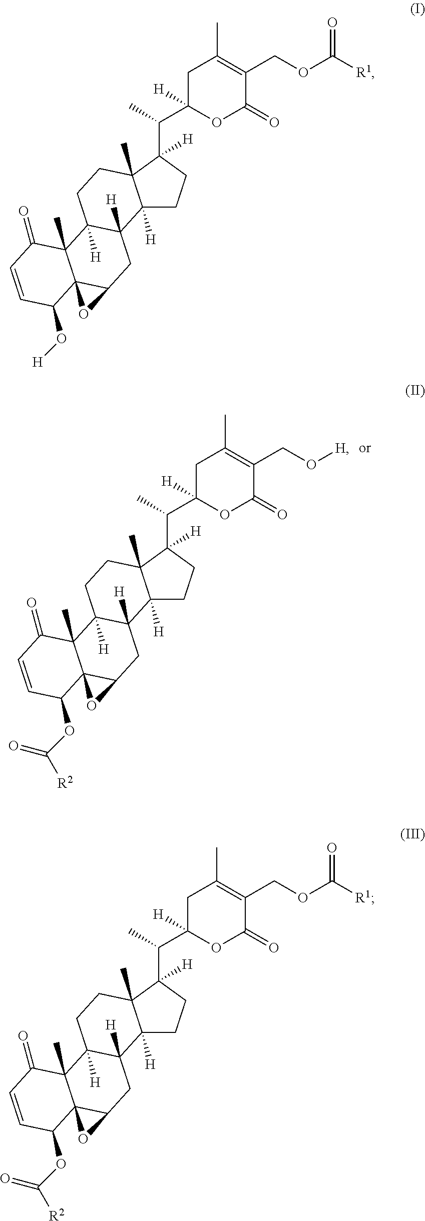

or a pharmaceutically acceptable salt thereof;
wherein:
R¹ and R² are independently selected from C₁-C₆ haloalkyl, N(R³)(R⁴), 3- to 6-membered monocyclic heterocyclyl, and 5- to 10-membered monocyclic or bicyclic heteroaryl, each of which may be optionally substituted with one or more (for example, 1, 2, 3, or 4) R⁵ groups;
R³ and R⁴ are independently selected at each occurrence from hydrogen, C₁-C₆ alkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, each of which may be substituted with one or more (for example 1, 2, 3, or 4) R⁵ groups;
Or R³ and R⁴ are brought together with the nitrogen to which they are attached to form a 3- to 6-membered monocyclic heterocycle ring optionally substituted with one or more R⁵ groups;
R⁵ is independently selected at each occurrence from halo, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀- C₃ alkyl)-, R^{z}C(O)—, R^{z}S(O)—, and R^{z}S(O)₂—;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more (for example, 1, 2, 3, or 4) Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR′, —SR^{x}, and —NR^{x}R^{y}, each of which may be optionally substituted with one or more (for example 1, 2, 3, or 4) Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

Also disclosed herein are pharmaceutical compositions comprising the compound of any preceding aspect.

The compounds and compositions disclosed shown herein are effective in treating, inhibiting, reducing, decreasing, ameliorating and/or preventing cancers, and/or metastasis in a subject comprising administering to the subject a therapeutically effective amount of the compound of any preceding aspect or the pharmaceutical composition of any preceding aspect. In some examples, the cancer is selected from the group consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, breast cancer, epithelial cancer, renal cancer, genitourinary cancer, esophageal carcinoma, head and neck carcinoma, large bowel cancer, hematopoietic cancers, testicular cancer, colon cancer, rectal cancer, prostate cancer, and pancreatic cancer.

In one aspect, disclosed herein are methods of treating, inhibiting, reducing, decreasing, ameliorating and/or preventing a neurological disorders (such as, for example, Alzheimer's disease (AD), Parkinson's disease (PD), Huntington's Disease, Amyotrophic Lateral Sclerosis, Multiple Sclerosis (MS), dentatorubropallidoluysian atrophy, Kennedy's disease, spinocerebellar ataxia, fragile X syndrome, fragile XE mental retardation, Friedreich's ataxia, myotonic dystrophy, spinocerebellar ataxia type 8, spinocerebellar ataxia type 12, Alexander disease, Alper's disease, ataxia telangiectasia, Batten disease, Canavan disease, Cockayne syndrome, corticobasal degeneration, Creutzfeldt-Jakob disease, ischemia stroke, Krabbe disease, Lewy body dementia, multiple system atrophy, Pelizaeus-Merzbacher disease, Pick's disease, primary lateral sclerosis, Refsum's disease, Sandhoff disease, Schilder's disease, spinal cord injury, spinal muscular atrophy (SMA), Steele-Richardson-Olszewski disease, or Tabes dorsalis) in a subject comprising administering to the subject a therapeutically effective amount of the compound of any preceding aspect or the pharmaceutical composition of any preceding aspect.

Also disclosed herein are methods for treating, inhibiting, reducing, decreasing, ameliorating and/or preventing infectious diseases (e.g., infectious diseases caused by infection of a virus, a bacterium, a fungus, or a parasite) in a subject comprising administering to the subject a therapeutically effective amount of the compound of any preceding aspect or the pharmaceutical composition of any preceding aspect.

In one aspect, disclosed herein are methods inhibiting, reducing, decreasing, ameliorating and/or preventing Notch1 signaling in a cell with increased levels of Notch1 signaling and/or activity comprising contacting the cell with a therapeutically effective amount of the compound of any preceding aspect or the pharmaceutical composition of any preceding aspect. In one aspect, the cell with increased Notch1 signaling and/or activity can be in a subject. Thus, in one aspect, disclosed herein are methods of treating, inhibiting, reducing, decreasing, ameliorating and/or preventing increased Notch1 signaling and/or activity in a subject with increased signaling and/or activity comprising administering to the subject a therapeutically effective amount of the compound of any preceding aspect or the pharmaceutical composition of any preceding aspect.

### DESCRIPTION OF DRAWINGS

FIG. 1A shows structure of ASR488. FIGS. 1B, 1C, and 1H show that TCCSUP and HT1376 cells were treated with the indicated concentration of ASR488 or vehicle (DMSO) for 24, 48, and 72 h, followed by MTT assay for assessing cell viability. FIG. 1D shows that ASR488-treated cells show up-regulation in the expression of Cleaved PARP and Bax in a time-dependent manner. FIG. 1E shows that ASR488-treated cells show down-regulation in the expression of Bcl2 and P65 in a time-dependent manner. FIG. 1F shows that apoptosis was quantified using flow cytometry of Annexin V-FITC and PI-stained, ASR488-treated TCCSUP cells. FIG. 1G shows representative dot blots of the apoptosis assay performed using flow cytometry of Annexin V-FITC and PI-stained, ASR488-treated TCCSUP cells. The Student's t-test was used to identify statistically significant differences between vehicle and treatment at each concentration. *p<0.05, **p<0.01 and ***p<0.001.

FIG. 2A shows distribution of DEGs demonstrated by Volcano diagram. The upregulated genes in ASR488-treated TCCSUP cells relative to TCCSUP cells treated with vehicle (DMSO) are presented in red, whereas the green dots represent the downregulated genes. The blue dots represent the genes that are without any significant diversity. FIG. 2B shows Venn diagram. The sum of the numbers in each large circle are the total number of genes in ASR488-treated or vehicle-treated TCCSUP cells, and the common genes among the samples are represented in the overlapping part. FIG. 2C shows that ASR488 treated TCCSUP cells subjected to immunoblotting and CPEB1 and IL11 genes were analyzed. FIG. 2D shows qRT-PCR analysis of top upregulated genes are displayed as fold difference between ASR488-treated TCCSUP cells and TCCSUP cells treated with vehicle (DMSO).

FIGS. 3A-3C show the gene ontology (GO) enrichment analysis. The top 20 downregulated enriched DEGs grouped into functional groups, presented as (FIG. 3A) biological process (BP), (FIG. 3B) cellular component (CC), and (FIG. 3C) molecular function (MF).

FIGS. 4A-4C show the gene ontology (GO) enrichment analysis. The top 20 upregulated enriched DEGs are grouped into functional groups, which have been presented as (FIG. 4A) biological process (BP), (FIG. 4B) cellular component (CC), and (FIG. 4C) molecular function (MF).

FIGS. 5A-5C show KEGG pathway analysis. Fisher exact test was used to perform KEGG pathway enrichment analysis. KEGG pathways (p<0.05) that are significantly enriched are presented as (FIG. 5A) Top 20 significantly enriched pathways in the KEGG enrichment analysis results. FIG. 5B shows the 20 enriched pathways that are significantly upregulated in the KEGG enrichment analysis results. FIG. 5C shows Top 20 significantly downregulated enriched pathways in the KEGG enrichment analysis results. For each KEGG pathway, the fold enrichment of the pathway is indicated by the bar.

FIGS. 6A-6C show reactome pathway analysis. The Reactome pathway enrichment analysis of DEGs. Twenty Pathways that were significantly affected are listed. These pathways include both the upregulated and downregulated pathways.

FIG. 7 shows Cluster Analysis Control (Treat) and ASR488 treated TCCSUP cells (Untreat). Hierarchical cluster of DEGs. Hierarchical cluster analysis for differential expression of genes using a fold change <0.5 or >2 and p<0.05. The color scale represent the relative levels of DEGs. Blue represents low levels, medium levels are represented by white while the red represents high levels. Samples represented are TCCSUP cells and ASR488-treated TCCSUP cells.

FIGS. 8A-8D show that advance bladder cancer has significantly low CPEB1 expression. ASR488 induces CPEB 1 expression in Bladder Cancer cells. Bladder cancer patient tissue samples were analyzed for CPEB 1 expression by immunohistochemistry and qPCR analysis CPEB 1 expression is low in bladder cancer patient cohort (FIGS. 8A-8B) and in advance bladder cancer cell lines (FIGS. 8C-8D).

FIGS. 9A-9E show that ASR488 induces CPEB1 mediated cell cycle arrest in bladder cancer cells. (FIG. 9A) The scanning flourimetery analysis of ASR458 and purified CPEB1 protein showed that ASR488 binds directly with CPEB1 and changes the Tm of bound form by 9° C. (FIG. 9B) ASR488 treatment induces p53 independent p27 upregulation in Bladder Cancer cells. (FIG. 9C) The result is GO/G1 cell cycle arrest in ASR488 treated Bladder Cancer Cells. (FIG. 9D) p27 upregulation is due to CPEB1 induction by overexpressing CPEB1 in TCCSUP cells. (FIG. 9E) CPEB1 overexpression significantly upregulated (13%; p<0.01) apoptosis in TCCSUP cells.

FIGS. 10A-10C show that ASR488 changes p27/Kip1 localization and induces apoptosis in bladder cancer cells. (FIGS. 10A-10B) Upregulation and nuclear localization of p27 in ASR488 treated cells induces GO/G1 cell cycle arrest and (FIG. 10C) subsequent apoptosis in TCCSUP cells.

FIGS. 11A-11B show that CPEB1 knockdown negates ASR488 induced cell death in bladder cancer cells. To analyze whether ASR488 treatment can induce p27 mediated cell death in CPEB1 knockdown bladder cancer cells, we knocked down CPEB1 with SiRNA in TCCSUP cells. (FIG. 11A) ASR488 treatment was unable to restore CPEB1 expression in CPEB1 knockdown cells. Moreover, P27 expression corresponded with CPEB1 expression pattern in the Knockdown cells. (FIG. 11B) ASR488 treatment did not induced cell death in CPEB1 knockdown cells. These results confirmed that ASR488 induced cell death is through activation of CPEB1/p27 signaling axis.

FIGS. 12A-12E show that change in p27 localization reduces invasive and migratory capability of advance bladder cancer cells. Low P27 expression has been directly linked with invasiveness of bladder cancer cells and also with different stages of bladder cancer. As ASR488 treatment increased CPEB1 mediated p27 expression, treatment effect on invasive and migratory potential of TCCSUP and HT1376 cells was analyzed. (FIGS. 12A-12C) The invasive and migratory potential were significantly reduced. (FIG. 12D). Moreover, overexpression of CPEB1 in TCCSUP cells also had similar effect on invasive and migratory potential of TCCSUP cells.

FIG. 13A-13I show that ASR490 specifically inhibits Notch1-mediated survival of CRC cells. (FIG. 13A) Structure of ASR490. (FIG. 13B & 13C) ASR490 or vehicle were used at indicated concentrations to treat HCT 116 and SW-620 cells for 24 h and 48 h followed by the MTT assay for cell viability. (FIG. 13D) Immunoblot analysis of cell lysates from HCT 116 and SW620 cells treated with the IC₅₀ concentration of ASR490 or vehicle (DMSO) for 12 and 24 h. (FIG. 13E) Docking study with Medusa Dock was performed with NRR domain and ASR490, the blue dotted lines (H-bond), Notch1-NRR domain with α-helices (cyan), (3-sheets (magenta), loops (salmon) and ASR490 (green licorice) are represented with water molecules (red spheres). (FIG. 13F) Protein run melt profile with temperature plotted against first derivative of fluorescence curve (−f/dt) and the lowest curve points taken at respective melting temperature (Tm) for NRR+ASR490 and NRR+Vehicle samples. (FIG. 13G) The CETSA assay was performed on ASR490-treated HCT 116 cells at the indicated temperatures followed by ELISA with NRR antibody and temperature was plotted against the absorbance (450 nm) changes. (FIG. 13H) Analysis of ASR490-treated cells for p65/Bcl-2 expression in a time-dependent manner. Data are presented as the mean±standard deviation (SEM/SD) of three independent experiments. (FIG. 13I) ASR490-treated cells show downregulation of Notch1 and HES1 transcripts in a time-dependent manner. Statistical significance between vehicle and treatment at each concentration was calculated with the Student's t-test. *p<0.05, **p<0.01 and ***p<0.001.

FIGS. 14A-14H show inhibition of Notch1-mediated oncogenic signaling in CRC cells (FIGS. 14A & 14B) HCT116 as well as SW620 cells treated with IC₅₀ concentration of ASR490 or vehicle, were stained with Annexin V-FITC and PI. Values, mean±SEM. *P<0.05; **P<0.01 (Student t test). (FIGS. 14C & 14D) Cell lysates from ASR490-treated HCT 116 and SW620 cells were analyzed for cleaved PARP and Bax expression. (FIGS. 14E & 14F) Migration assay was performed for HCT 116 and SW620 cells that are treated with ASR490 and vehicle. Values, mean±SEM. *P<0.05; **P<0.01 (Student t test). (FIGS. 14G & 14H) Immunoblotting analysis of ASR490-treated and vehicle-treated HCT 116 and SW620 cell lysates for E-cadherin, N-cadherin, and β-catenin expression.

FIGS. 15A-15E show that ASR490 overcomes Notch1 overexpression and inhibits the growth of HCT/Notch1 transfectants. (FIG. 15A) Western blot analysis of Notch1 basal expression in HCT116 (pCMV-transfected and Notch1-transfected) cells. (FIG. 15B) For assessment of cell viability with MTT assay, IC₅₀ concentration of ASR490 or Vehicle (DMSO) was used to treat HCT116 stable transfectants C₄ and C₅ for 24 h. One way ANOVA with multiple comparison test was used to calculate the statistical significance between different experimental groups. *p<0.05 and **p<0.01. (FIG. 15C) Colony-forming assay on pCMV/HCT116 (vector transfected) and HCT116 stable transfectants C₄ and C₅ treated with ASR490 or vehicle (DMSO) were performed. All experiments were performed in triplicate. One way ANOVA with multiple comparison test was used to calculate the statistical significance between different experimental groups. *p<0.05 and **p<0.01. (FIG. 15D) Immunoblot analysis of ASR490-treated pCMV/HCT, C₄, and C₅ cells for Notch1 and HES1 expression in a time-dependent manner (12 and 24 h). (FIG. 15E) Densitometry analysis was performed with ImageJ software for the immunoblots. Values, mean±SEM. *P<0.05; **P<0.01 (Student t test).

FIGS. 16A-16D show suppression of Notch1-mediated survival and induction of apoptosis in Notch1 transfectants. (FIG. 16A) pCMV/HCT116 and stable clones (C₄ and C₅) were treated with the indicated concentration of ASR490 or vehicle (DMSO) for the indicated time points and total protein lysates were analyzed for the pro-survival markers NF-κB (p65) and Bcl-2. (FIG. 16B) Densitometry analysis was performed with ImageJ software for the immunoblots. Values, mean±SEM. Statistical significance between vehicle and treatment at each concentration was calculated with the Student's t-test. *p<0.05. (FIG. 16C) Total protein lysates from ASR490-treated pCMV/HCT 116, C₄, and C₅ cells were analyzed for expression of the pro-apoptotic markers cleaved PARP and Bax. (FIG. 16D) FACS analysis was performed (Annexin V-FITC and PI staining) in non-transfected and Notch1-overexpressing HCT116 transfectants that were treated with the IC₅₀ concentration of ASR490 or vehicle (DMSO). Values, mean±SEM. *P<0.05; **P<0.01 (Student t test).

FIGS. 17A-17D show that ASR490 overcomes Notch1-induced EMT and decreases tumorigenicity of CRC cells. (FIG. 17A) A trans-well invasion assay was performed for pCMV/HCT and Notch1 overexpressing HCT116 transfectants (C₄ and C₅) that were treated with either ASR490 or vehicle (DMSO). (FIG. 17B) Migration assays were performed in vector-transfected and Notch1-overexpressing cells (C₄ and C₅) that were treated with ASR490 or vehicle. Analysis was carried out with Image J software and values are presented as mean±SEM. *P<0.05; **P<0.01 (Student t test). (FIG. 17C) Cell lysates from ASR490-treated and vehicle-treated pCMV/HCT, C₄, and C₅ cells were subjected to western blot analysis using E-cadherin, N-cadherin, Snail, 0-catenin, MMP-9, and Snail antibodies. (FIG. 17D) Densitometry analysis was performed with ImageJ software for the immunoblots. Values plotted are mean±SEM. Statistical significance between vehicle and treatment at each concentration was calculated by the Student's t-test. *p<0.05 and **p<0.01.

FIGS. 18A-18C show that ASR490 reduces Notch1-mediated tumor burden in xenografts pCMV/HCT116 and C₄ (1×10⁶) were injected subcutaneously into separate flanks of the mice (n=6-8). ASR490 (5 mg/kg) or 1% DMSO (Vehicle) (100 μl volume) was administered intraperitoneally thrice a week. (FIG. 18A) Weekly thrice the tumor volume (mm3) was measured in both ASR490 and vehicle treated mice. Mean tumor volumes±SEM are shown. *p<0.05 and **p<0.01 by two-tailed Student t test. (FIG. 18B) IHC analysis of Ki-67, Notch1, HES1, and NFκB (p65) (FIG. 18C). Protein isolated from tissue samples taken from HCT/Notch1 xenografts was subjected to immunoblot analysis with Notch1 and HES1 antibodies.

FIG. 19A shows that synthesis scheme for ASR490. FIG. 19B shows that HCT 116 and SW620 cells were treated with the IC₅₀ concentration of ASR490 or vehicle (DMSO) for 3, 6, 12 and 24 h and total cell lysates were subjected to immunoblotting with Notch1 and HES1 antibodies. FIG. 19C that HCT 116 cells were treated with the IC₅₀ concentration of ASR490 or vehicle (DMSO) for 12 h and 24 h and extracted RNA were subjected to qPCR with Notch1 and HES1 specific primers. Data are presented as the mean±standard deviation (SEM/SD) of three independent experiments. The Student's t-test was used to calculate the statistical significance between the vehicle and treatment at each concentration. *p<0.05 and **p<0.01. FIG. 19D shows that HCT 116 cells were treated with the IC₅₀ concentration of ASR490 or vehicle (DMSO) for 12 h and 24 h and total cell lysates were subjected to immunoblotting with Notch2 and Notch3 antibodies.

FIG. 20A shows that the whole cell lysates of HCT116/pCMV and HCT116/Notch1-cells was subjected to immunoblotting with Notch1 9NICD) antibody. FIG. 20B shows MTT assay of HCT116/pCMV and HCT116/Notch1-cells treated with indicated concentrations of ASR490 or vehicle for 24 h. Six biological replicates used for MTT assays and it was repeated twice for each experiment. Data are presented, from independent experiments, as the mean±standard deviation (SEM/SD). The Student's t-test was used to calculate the statistical significance between the vehicle and treatment at each concentration. *p<0.05, **p<0.01 and ***p<0.001. FIG. 20C shows that whole cell lysates from bladder cancer cells was subjected to western blotting and basal level of Notch1 (NICD) was analyzed. FIG. 20D shows MTT assay of TCCSUP and HT1376 cells treated with the indicated concentration of ASR490 or vehicle (DMSO) for 24 h. Six biological replicates used for MTT assays and it was repeated twice for each experiment. Data are presented, from independent experiments, as the mean±standard deviation (SEM/SD). The Student's t-test was used to calculate the statistical significance between the vehicle and treatment at each concentration. *p<0.05 and # Not significant.

FIGS. 21A-21D show that ASR490 abrogates Breast Cancer Stem Cell (BCSC) growth by targeting Notch1 signaling. Earlier studies have shown that ASR490 targets NRR region of Notch and downregulates Notch1 signaling (PMID: 33087513). Studies have shown that Notch1 signaling is mainstay of breast cancer stem cell (BCSC) growth. The effect of ASR490 was assessed on both ALDH++(BCSC) and ALDH−−(BC) cells and cell viability assays showed that ASR490 effectively suppressed ALDH+ cells' growth (IC 50: 770 nM at 24 h, and 443 nM at 48 h (compared to its effects on ALDH cells (IC 50: 1.6 μM at 24 h and 836 nM at 48 h (FIG. 21A). Immunoblot analysis was performed to determine the molecular mechanism by which ASR490 inhibited the growth of BCSCs. Results showed significant time dependent downregulation of NICD expression and its downstream effector HES1 protein in ALDH+ and ALDH lysates following treatment with ASR490 (FIG. 21B). Moreover, the downregulation of pro survival genes (p65 & BCl 2; FIG. 21C).

FIGS. 22A-22E show that ASR490 inhibits the stem cell attributes such as Sphere forming, colony forming and invasive ability of BCSCs. Spheroid formation and colonogenic abilities are major attributes of stem cells that impart invasive behavior to these cells. ASR490 treatment significantly reduced the sphere formation (FIG. 22A) as well as colonogenic ability (FIG. 22B) of both ALDH+ and ALDH− cells. As shown in FIG. 22A, ALDH+ spheres expressed higher NICD expression levels, and ASR490 treated ALDH+ spheres showed a significantly lesser expression of NICD. Although, a lower expression of NICD was observed in ALDHBC cells, after ASR490 treatment, it was entirely abolished. Migration and invasion assays (FIGS. 22C, 22D) revealed that ASR490 significantly decreased the migratory and invasive ability of ALDH+ and ALDH− cells. (FIG. 22E) The effect of ASR490 was found to be more profound on suppressing the migratory and invasive capabilities of ALDH+ cells than the ALDH− cells by suppressing mesenchymal marker b-catenin and inducing epithelial marker E-cadherin.

FIG. 23 shows that ASR490 induces ubiquitination of the intracellular Notch1 domain (NICD) by binding to NRR domain of Notch1. ASR 490 treatment can be inducing degradation of NRR of Notch 1 albeit independent of its transcription in ALDH cells. ASR 490 can induce NRR degradation by the ubiquitin pathway. ASR 490 being another proteasome inhibitor like MG-132 was analyzed by measuring proteasomal activity using a chymotrypsin-like compound with a 7-amido-4-methylcoumarin (AMC)-tagged peptide substrate an induction of proteasome activity was seen at 15 and 30 min and no significant changes were noted in ASR 490 treated ALDH+ cells (FIG. 23A). Commercially available positive and negative controls were used for these experiments. These results indicate that ASR 490 not a proteasomal inhibitor. Then, we analyzed whether ASR 490 induce ubiquitination of NRR a time dependent increased ubiquitin expression was seen in ASR 490 treated ALDH+ cells (FIG. 23B) and MG-132 was used as a positive control for our experiment, which showed a higher level of ubiquitin. Next, ubiquitination-associated NRR of Notch 1 degradation were examined. ALDH+ cells were treated with MG-132, ASR490 or in combinations and the cell lysates were IPed with NRR of Notch1 and western blot with ubiquitin antibody ASR490 induced NRR ubiquitination in ALDH+ cells.

FIGS. 24A-24C show that ASR490 reduces tumor burden in ALDH+ xenotransplanted mice by downregulating the NICD and proliferation markers. (FIG. 24A) ASR490 administration significantly reduced tumor burden in ALDH+ xenotransplanted mice. Moreover, (FIGS. 24B, 24C) the proliferative markers and ICN-Notch1 expression was also downregulated in tumors from mice administered with ASR490.

FIGS. 25A-25D show that ASR490 abrogates Triple Negative Breast Cancer (TNBC) cell growth by targeting Notch1 signaling. ASR 490 effectively suppressed growth (FIG. 25A) and colony formation ability (FIG. 25B) of TNBC cells (MDA-MB-231). IC₅₀ of ASR 490 is in nanomolar concentrations 760 nM at 24 h and 444 nM at 48 h). Apoptosis assays (FIG. 25C) confirmed that ASR 490 induced approximately 20 and 43 of apoptosis in MDA MB-231 cells at 24 and 48 h, respectively. Immunoblot results (FIG. 25D) showed a significant time-dependent downregulation of NICD expression and its downstream effector HES 1 in MDA-MB-231 cells following treatment with ASR 490. Downregulation of pro-survival genes (p65 and BCl-2) and concomitant induction of pro-apoptotic machinery were observed in ASR490 treated TNBC cells *p 0.001, p<0.002, **** 0.0001.

FIGS. 26A-26C show that Doxorubicin inhibits TNBC cell growth but induces Notch1 signaling. As a proof of principle, DOX was selected for the preliminary experiments. (FIG. 26A) Western blots were performed to confirm DNA damage potential of DOX by analyzing expression profiles of phosphorylated Ataxia telangiectasia mutated (ATM) and Ataxia telangiectasia and Rad3 related (ATR) in DOX treated MDA-MB-231 TNBC cells. Increased expressions of apoptotic marker cleaved PARP, and a concomitant downregulation of BCL-2 expression was also observed. (FIG. 26B) Results also demonstrated an induction of Notch 1 activation, as observed by increased levels of NICD and HES 1 compared to vehicle treated controls. (FIG. 26C) Effects of chemotherapeutic agents (Doxorubicin [DOX], Docetaxel [DTX], Fluorouracil [5-FU]) on inhibiting the growth of MDA-MB-231 TNBC cells was examined using cell viability assays and the IC₅₀ concentrations listed in FIG. 26C were found to effectively suppress cell growth.

FIGS. 27A-27B show that ASR490 sensitizes TNBC cells for Doxorubicin treatment and synergistically inhibits TNBC cell growth at low doses. Cell viability assays was used to determine the effects of IC₅₀ of ASR490 in combination with various doses ( 1/10th, 1/20th, 1/40th, and 1/80th of the IC₅₀) of DOX in inhibiting TNBC cell proliferation. Similarly, the effect of various concentrations of ASR490 (⅕th, 1/10th, 1/15th, 1/20th, and 1/25th of the IC₅₀ dose) in combination with the IC₅₀ concentration of DOX was performed. Synergistic effects of combinations were calculated via isobologram analysis. Based on this data, the ASR490 (⅕th of IC₅₀) and DOX ( 1/20th of IC₅₀) concentrations were selected, which demonstrated the best synergy in each experiment. Cell viability assays and isobologram analysis showed that treatment with ⅕th of IC₅₀ of ASR490 (152 nM) in combination with 1/20th of IC₅₀ of DOX (46.4 nM) had a synergistic effect in inhibiting proliferation of MDA-MB0231 TNBC cells (30% inhibition; p<0.001) FIGS. 27A, 27B). In contrast, treatment with ⅕th of IC₅₀ of ASR490 and 1/20th of IC₅₀ of DOX alone did not inhibit the growth of the MDA-MB-231 cells.

FIG. 28 shows that a combination of low doses of ASR490 and Doxorubicin works synergistically to downregulate Notch1 signaling. Western blots demonstrated that while neither ⅕th of ASR490 nor 1/20th of DOX treatment altered expression of their targets, their combination significantly inhibited NICD and downstream HES1 expression, as well as downregulated the expression of pro survival markers, while increasing cleaved PARP expression. This combination also retained the DNA damaging effect of DOX as determined by elevated expression of phos ATR^{Ser428} and phos ATM^{Ser1981}. As observed in the cell viability assays, treatment with ⅕th of IC₅₀ of ASR490 and 1/20th of IC₅₀ of DOX alone did not affect DNA damaging response of the TNBC cells.

FIGS. 29A-29E show that ASR458 inhibits p53 wild type (HCT116) and p53 mutant (SW620) cell growth. A small molecule library was screened and ASR458 was identified (FIG. 29A), a novel small molecule inhibitor, that inhibits p53 wt HCT116 (IC₅₀; 24 h 950 nM, 48 h 935 nM, 72 h 620 nM) (FIG. 29B) as well as p53mut SW620 (IC50; 24 h 1.2 μM, 48 h 620 nM, 72 h 250 nM) (FIG. 29C) CRC cell growth with IC₅₀ in nM concentrations. The pro apoptotic markers were significantly induced in ASR458 treated HCT116 cells (FIG. 29D). Furthermore, an annexin/FITC apoptosis assay confirmed over 30% apoptosis in ASR458 treated HCT116 cells (FIG. 29E). To restore cellular homeostasis in stress conditions p53 induces apoptosis through p21.

FIGS. 30A-30D show that ASR458 treatment restored p53 function in HCT116 cells and induced a time dependent increase in expression of p21 thereby causing a cell cycle arrest (FIGS. 30A-30B). However, in SW620, a R273H p53mut cell line, ASR458 treatment did not activate p53 function as indicated by a time dependent decrease in p21 expression (FIGS. 30C-30D).

FIGS. 31A-31D show that ASR458 overcomes AKT induces pro-survival signaling in HCT116 cells. (FIG. 31A) ASR458 overcomes AKT overexpression in HCT116 cells and significantly downregulates cell survival markers in ASR458 treated pCMV/HCT116, AKT6, AKT12 cells. (FIG. 31B) ASR458 treatment inhibits cell growth in HCT116 (400 nm), AKT6 (750 nm) and AKT12 (750 nm). (FIG. 31C) ASR458 inhibits cell growth in pCMV/HCT116, AKT6, AKT12 by inducing expression of pro-apoptotic marker such as C1 PARP. (FIG. 31D) The ASR458 treated pCMV/HCT116, AKT6, AKT12 cells were stained with Propidium Iodide and FITC and subjected the cells to FACS analysis. The treatment was able to induce significant apoptosis (p<0.05) in all the pCMV/HCT116, AKT6, AKT12 cells.

FIGS. 32A-32E show that ASR458 overcomes AKT induced colonogenic and invasive capability of HCT116 cells. (FIGS. 32A, 32B) AKT overexpression resulted in higher number of colonies in AKT 6 AKT 12 cells ASR 458 treatment overcame the AKT induced colony forming ability AKT 6 AKT 12 cells. (FIGS. 32C, 32D) Invasive capacity of pCMV/HCT 116 AKT 6 AKT 12 was abrogated and (FIG. 32E) EMT markers were downregulated in ASR 458 treated pCMV/HCT116 AKT 6 and AKT 12 cells.

FIGS. 33A-33C show that ASR458 inhibits tumor growth in HCT116, and HCT/AKT xenograft mice models by inhibiting key survival and cell proliferation regulators. (FIGS. 33A-33B) ASR 458 treatment reduced tumor burden in HCT 116 and AKT overexpressing HCT 116 cells. (FIG. 33C) IHC analysis showed that ASR 458 treatment overcame pAKT and Notch 1 overexpression and downregulated pro survival signaling in AKT overexpressing HCT 116 cells.

FIGS. 34A-34F show that ASR458 induces high ROS and ER stress in p53 mut SW620 cells In SW620 cells (p53 mutant CRC), (FIG. 34A) ASR458 treatment induced ROS and (FIG. 34B) subsequent ER stress when the cells were stained with calnexin dye. (FIG. 34C) ASR458 treatment induced ER stress signaling (i.e., phosphorylation of eIF2 α) in p53 mut SW620 cells, which triggered ATF4 activation and (FIG. 34E) subsequent induction of cascade of autophagy events (ATG5, LC3B and Lamp1), (FIG. 34F) causing formation of autophagosomes and autophagy mediated cell death.

FIG. 35 shows that ATF4 mediates ASR458 induced autophagy in SW620 cells. Silencing ER stress marker ATF 4, a key regulator of autophagy, caused resistance to ASR458 and abrogated autophagy signaling in SW620 cells. This suggested that induction of ER stress is critical for the cytotoxic effects of ASR458 in p53 mut CRC. Preliminary knockdown studies indicate that silencing of ER markers causes resistance to ASR458 in vitro, further confirming ER stress as the mechanism of ASR458 action in p53 mut CRC.

FIGS. 36A-36B show that ASR458 inhibits tumor growth of HCT116 and SW620 Xenografted mice. (FIG. 36A) ASR458 inhibits tumor growth in HCT116 and SW620 Xenografts. (FIG. 36B) IHC analysis of HCT116 and SW620 for proliferation marker.

FIGS. 37A-37F show that AKT negatively regulates ATF4 mediated autophagy. (FIG. 37A) Total protein lysates from ASR-treated SW620 cells were used for western blotting to analyze the effect on Pakt expression at indicated time points. (FIG. 37B) Western Blot analysis of AKT overexpressing and ASR458 treated SW620 cells. (FIG. 37C) ASR458 treated AKT overexpressing SW620 transfectants were subjected to ROS analysis (FIG. 37D) SW620 cells and AKT transfectants were treated with IC₅₀ dose of ASR458 or vehicle (DMSO) and MTT assay was performed (FIG. 37E) Confocal imaging of vehicle treated and ASR458 treated AKT overexpressing SW620 cells for ER stress (ER-ID® Green assay kit—Enzo Life Sciences) (FIG. 37F) Autophagy signaling is not activated in HCT116.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

The terms “patient,” “subject” and “individual” are used interchangeably herein, and mean an animal (e.g., mammalian (such as human, equine, bovine, ovine, porcine, canine, etc.), reptilian, piscine, etc.) to be treated, diagnosed and/or to obtain a biological sample from.

As used herein, “bind,” “binds,” or “interacts with” means that one molecule recognizes and adheres to a particular second molecule in a sample or organism, but does not substantially recognize or adhere to other structurally unrelated molecules in the sample. Generally, a first molecule that “specifically binds” a second molecule has a binding affinity greater than about 10⁸ to 10¹² moles/liter for that second molecule and involves precise “hand-in-a-glove” docking interactions that can be covalent and noncovalent (hydrogen bonding, hydrophobic, ionic, and van der Waals).

As used herein, “protein” and “polypeptide” are used synonymously to mean any peptide-linked chain of amino acids, regardless of length or post-translational modification, e.g., glycosylation or phosphorylation.

By the term “gene” is meant a nucleic acid molecule that codes for a particular protein, or in certain cases, a functional or structural RNA molecule.

As used herein, a “nucleic acid” or a “nucleic acid molecule” means a chain of two or more nucleotides such as RNA (ribonucleic acid) and DNA (deoxyribonucleic acid).

As used herein, the terms “therapeutic,” and “therapeutic agent” are used interchangeably, and are meant to encompass any molecule, chemical entity, composition, drug, cell(s), therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, cells, natural or synthetic compounds and the like.

As used herein, the term “treatment” is defined as the application or administration of a therapeutic agent to a patient or subject, or application or administration of the therapeutic agent to an isolated tissue or cell line from a patient or subject, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, secbutyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Haloalkoxy” indicates a haloalkyl group as defined herein attached through an oxygen bridge (oxygen of an alcohol radical).

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydroisoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 3, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfone, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, Pa., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

In one aspect, a compound is provided of Formula I, Formula II, or Formula III:

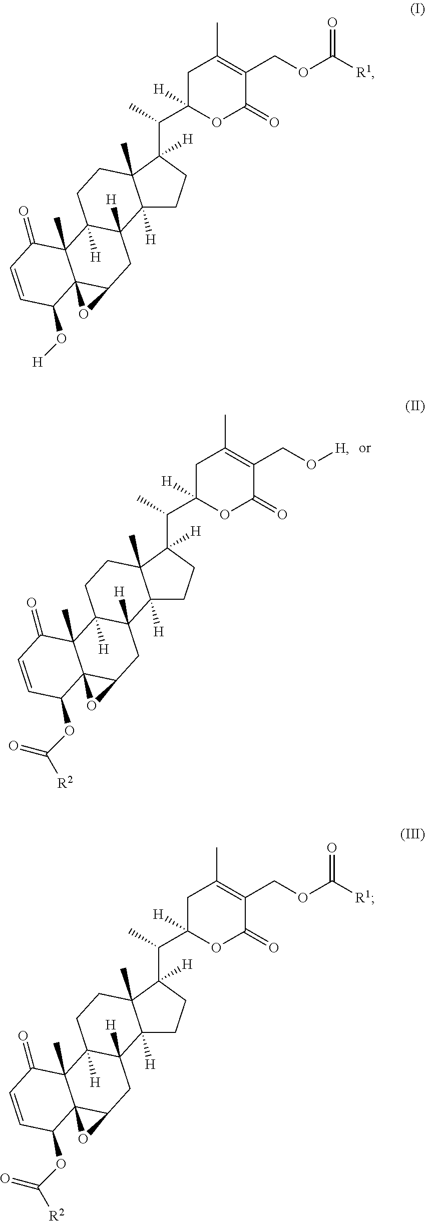

or a pharmaceutically acceptable salt thereof;
wherein:
R¹ and R² are independently selected from C₁-C₆ haloalkyl, N(R³)(R⁴), 3- to 6-membered monocyclic heterocyclyl, and 5- to 10-membered monocyclic or bicyclic heteroaryl, each of which may be optionally substituted with one or more (for example, 1, 2, 3, or 4) R⁵ groups;
R³ and R⁴ are independently selected at each occurrence from hydrogen, C₁-C₆ alkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, each of which may be substituted with one or more (for example 1, 2, 3, or 4) R⁵ groups;
Or R³ and R⁴ are brought together with the nitrogen to which they are attached to form a 3- to 6-membered monocyclic heterocycle ring optionally substituted with one or more R⁵ groups;
R⁵ is independently selected at each occurrence from halo, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀- C₃ alkyl)-, R^{z}C(O)—, R^{z}S(O)—, and R^{z}S(O)₂—;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more (for example, 1, 2, 3, or 4) Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more (for example 1, 2, 3, or 4) Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some embodiments of Formula I or Formula III, R¹ is selected from C₁-C₃ fluoroalkyl or C₁-C₃ haloalkyl. In some embodiments of Formula I or Formula III, R¹ is dichloromethyl.

In some embodiments of Formula I or Formula III, R¹ is N(R³)(R⁴). In some embodiments of Formula I or Formula III, R¹ is 3- to 6-membered monocyclic heterocyclyl optionally substituted with 1, 2, 3, or 4 R⁵ groups as allowed by valency. In some embodiments of Formula I or Formula III, R¹ is 5- to 10-membered monocyclic or bicyclic heteroaryl optionally substituted with 1, 2, 3, or 4 R⁵ groups as allowed by valency.

In some embodiments of Formula II or Formula III, R² is selected from C₁-C₃ fluoroalkyl or C₁-C₃ haloalkyl. In some embodiments of Formula II or Formula III, R² is dichloromethyl.

In some embodiments of Formula II or Formula III, R² is N(R³)(R⁴). In some embodiments of Formula II or Formula III, R² is 3- to 6-membered monocyclic heterocyclyl optionally substituted with 1, 2, 3, or 4 R⁵ groups as allowed by valency. In some embodiments of Formula II or Formula III, R² is 5- to 10-membered monocyclic or bicyclic heteroaryl optionally substituted with 1, 2, 3, or 4 R⁵ groups as allowed by valency.

In some embodiments of Formula I, Formula II, or Formula III, R³ is hydrogen. In some embodiments of Formula I, Formula II, or Formula III, R³ is C₁-C₃ alkyl. In some embodiments of Formula I, Formula II, or Formula III, R³ is methyl.

In some embodiments of Formula I, Formula II, or Formula III, R⁴ is hydrogen. In some embodiments of Formula I, Formula II, or Formula III, R⁴ is C₁-C₃ alkyl. In some embodiments of Formula I, Formula II, or Formula III, R⁴ is methyl.

In some embodiments of Formula I, Formula II, or Formula III, R³ and R⁴ are each methyl.

In some embodiments of Formula I or Formula III, R¹ is selected from pyrrolyl, furanyl, thienyl, pyridyl, benzofuranyl, or quinolinyl optionally substituted with 1, 2, 3, or 4 R⁵ groups as allowed by valency.

In some embodiments of Formula I or Formula III, R¹ is

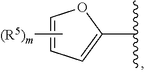

wherein m is 0, 1, 2, or 3.

In some embodiments of Formula I or Formula III, R¹ is

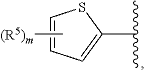

wherein m is 0, 1, 2, or 3.

In some embodiments of Formula I or Formula III, R¹ is

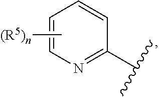

wherein n is 0, 1, 2, 3, or 4.

In some embodiments of Formula I or Formula III, R¹ is

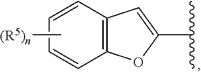

wherein n is 0, 1, 2, 3, or 4.

In some embodiments of Formula I or Formula III, R¹ is

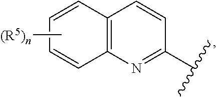

wherein n is 0, 1, 2, 3, or 4.

In some embodiments of Formula II or Formula III, R² is selected from pyrrolyl, furanyl, thienyl, pyridyl, benzofuranyl, or quinolinyl optionally substituted with 1, 2, 3, or 4 R⁵ groups as allowed by valency.

In some embodiments of Formula II or Formula III, R² is

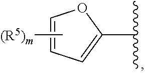

wherein m is 0, 1, 2, or 3.

In some embodiments of Formula II or Formula III, R² is

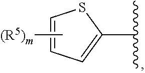

wherein m is 0, 1, 2, or 3.

In some embodiments of Formula II or Formula III, R² is

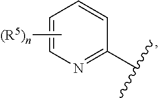

wherein n is 0, 1, 2, 3, or 4.

In some embodiments of Formula II or Formula III, R² is

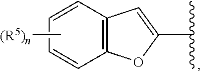

wherein n is 0, 1, 2, 3, or 4.

In some embodiments of Formula II or Formula III, R² is

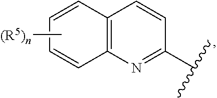

wherein n is 0, 1, 2, 3, or 4.

Representative examples of compounds of the present disclosure include, but are not limited to:

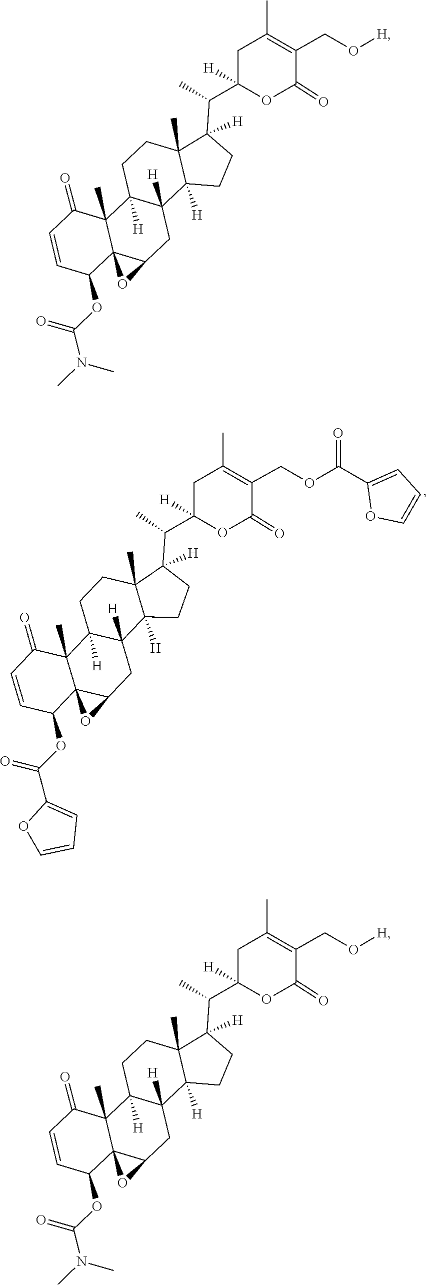

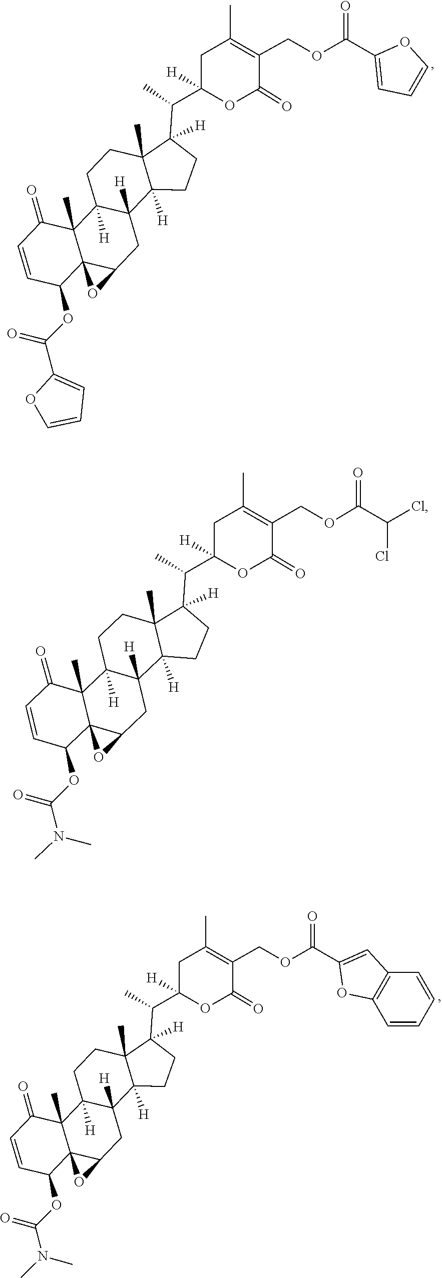

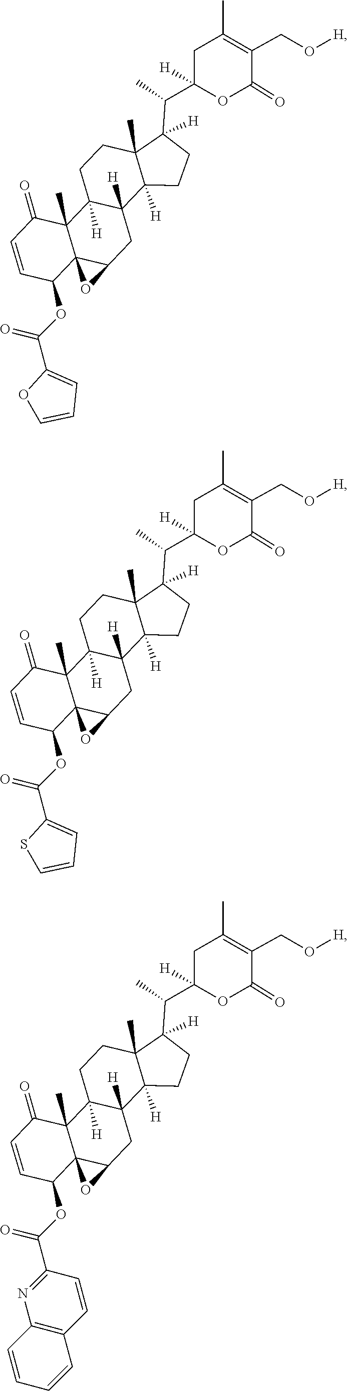

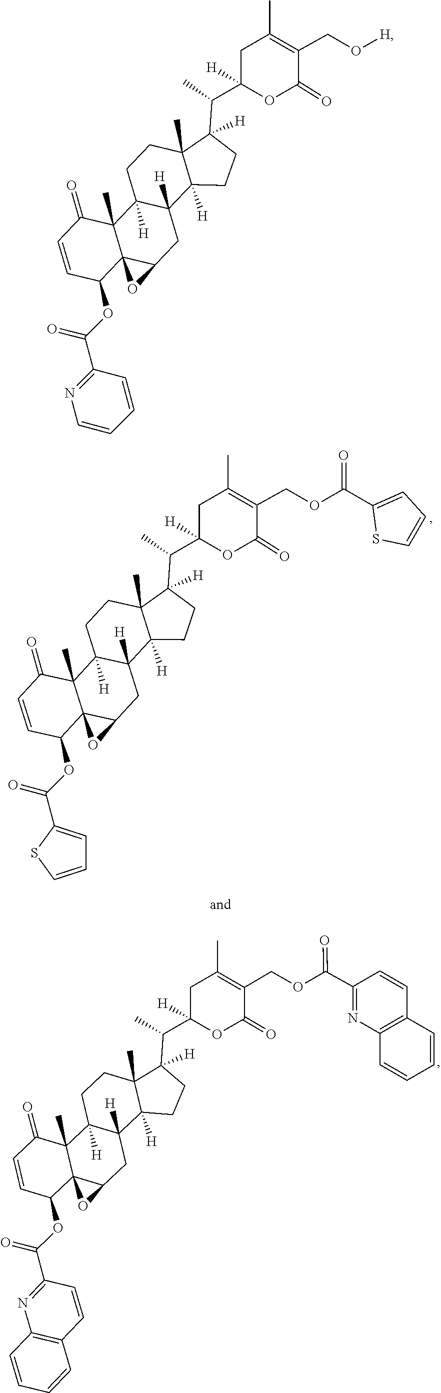

or a pharmaceutically acceptable salt thereof 103. The present disclosure also includes compounds of Formula I, Formula II, or Formula III with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d6-acetone, or d6-DMSO. A solvate can be in a liquid or solid form.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment, inhibition, decrease, reduction, amelioration, and/or prevention of a cancer, an infection, or a neurological disorder or any disorder with elevated levels of Notch1 signaling and/or activity in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an antioxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### Methods

It should be understood and herein contemplated that the composition disclosed herein can inhibit Notch1 activation. Binding of ligands to the extracellular domain of Notch1 triggers activation, driving sequential cleavages of the receptor, dimerization and translocation of the Notch receptor intracellular domain (NICD) to the nucleus to induce transcriptional activation. Notch1 activation triggers significant oncogenic signaling that manifests as enhanced metastatic potential and tumorigenesis in a variety types of cancers. The current landscape of Notch1 inhibitors, particularly gamma secretase inhibitors (GSI) such as LY-411,575 or DAPT, can have unintended biological implications (toxic and harmful side effects) because of broad substrate profile of gamma secretase. Recently, mAbs have been used to target NRR region in order to stabilize the region and prevent ligand-independent activation and wild-type Notch1 activation and thus decrease in NICD expression. However, small molecules that inhibit Notch1 activation through targeting negative regulatory region (NRR), has not been reported. The compositions disclosed herein target NRR region of Notch1 and inhibit NICD dimerization. The compositions disclosed herein exhibit low toxicity and improved efficacy.

Accordingly, in one aspect, disclosed herein are method of treating/inhibiting/reducing cancer in a subject comprising administering to the subject a therapeutically effective amount of the compound or the pharmaceutical composition disclosed herein. In some embodiments, the compound is provided of Formula I, Formula II, or Formula III:

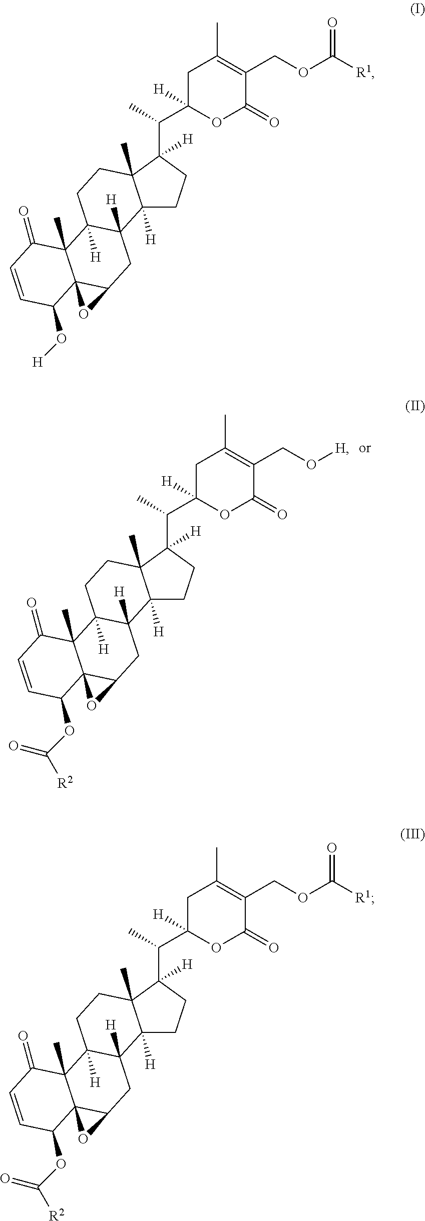

or a pharmaceutically acceptable salt thereof;
wherein:
R¹ and R² are independently selected from C₁-C₆ haloalkyl, N(R³)(R⁴), 3- to 6-membered monocyclic heterocyclyl, and 5- to 10-membered monocyclic or bicyclic heteroaryl, each of which may be optionally substituted with one or more (for example, 1, 2, 3, or 4) R⁵ groups;
R³ and R⁴ are independently selected at each occurrence from hydrogen, C₁-C₆ alkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, each of which may be substituted with one or more (for example 1, 2, 3, or 4) R⁵ groups;
Or R³ and R⁴ are brought together with the nitrogen to which they are attached to form a 3- to 6-membered monocyclic heterocycle ring optionally substituted with one or more R⁵ groups;
R⁵ is independently selected at each occurrence from halo, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀- C₃ alkyl)-, R^{z}C(O)—, R^{z}S(O)—, and R^{z}S(O)₂—;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more (for example, 1, 2, 3, or 4) Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more (for example 1, 2, 3, or 4) Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some embodiments of Formula I or Formula III, R¹ is selected from C₁-C₃ fluoroalkyl or C₁-C₃ haloalkyl. In some embodiments of Formula I or Formula III, R¹ is dichloromethyl.

In some embodiments of Formula I or Formula III, R¹ is N(R³)(R⁴). In some embodiments of Formula I or Formula III, R¹ is 3- to 6-membered monocyclic heterocyclyl optionally substituted with 1, 2, 3, or 4 R⁵ groups as allowed by valency. In some embodiments of Formula I or Formula III, R¹ is 5- to 10-membered monocyclic or bicyclic heteroaryl optionally substituted with 1, 2, 3, or 4 R⁵ groups as allowed by valency.

In some embodiments of Formula II or Formula III, R² is selected from C₁-C₃ fluoroalkyl or C₁-C₃ haloalkyl. In some embodiments of Formula II or Formula III, R² is dichloromethyl.

In some embodiments of Formula II or Formula III, R² is N(R³)(R⁴). In some embodiments of Formula II or Formula III, R² is 3- to 6-membered monocyclic heterocyclyl optionally substituted with 1, 2, 3, or 4 R⁵ groups as allowed by valency. In some embodiments of Formula II or Formula III, R² is 5- to 10-membered monocyclic or bicyclic heteroaryl optionally substituted with 1, 2, 3, or 4 R⁵ groups as allowed by valency.

In some embodiments of Formula I, Formula II, or Formula III, R³ is hydrogen. In some embodiments of Formula I, Formula II, or Formula III, R³ is C₁-C₃ alkyl. In some embodiments of Formula I, Formula II, or Formula III, R³ is methyl.

In some embodiments of Formula I, Formula II, or Formula III, R⁴ is hydrogen. In some embodiments of Formula I, Formula II, or Formula III, R⁴ is C₁-C₃ alkyl. In some embodiments of Formula I, Formula II, or Formula III, R⁴ is methyl.

In some embodiments of Formula I, Formula II, or Formula III, R³ and R⁴ are each methyl.

In some embodiments of Formula I or Formula III, R¹ is selected from pyrrolyl, furanyl, thienyl, pyridyl, benzofuranyl, or quinolinyl optionally substituted with 1, 2, 3, or 4 R⁵ groups as allowed by valency.

In some embodiments of Formula I or Formula III, R¹ is

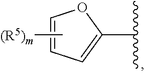

wherein m is 0, 1, 2, or 3.

In some embodiments of Formula I or Formula III, R¹ is

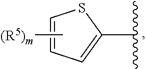

wherein m is 0, 1, 2, or 3.

In some embodiments of Formula I or Formula III, R¹ is

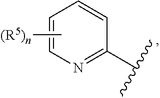

wherein n is 0, 1, 2, 3, or 4.

In some embodiments of Formula I or Formula III, R¹ is

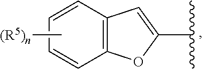

wherein n is 0, 1, 2, 3, or 4.

In some embodiments of Formula I or Formula III, R¹ is

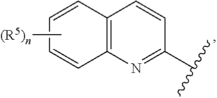

wherein n is 0, 1, 2, 3, or 4.

In some embodiments of Formula II or Formula III, R² is selected from pyrrolyl, furanyl, thienyl, pyridyl, benzofuranyl, or quinolinyl optionally substituted with 1, 2, 3, or 4 R⁵ groups as allowed by valency.

In some embodiments of Formula II or Formula III, R² is

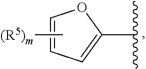

wherein m is 0, 1, 2, or 3.

In some embodiments of Formula II or Formula III, R² is

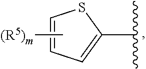

wherein m is 0, 1, 2, or 3.

In some embodiments of Formula II or Formula III, R² is

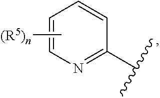

wherein n is 0, 1, 2, 3, or 4.

In some embodiments of Formula II or Formula III, R² is

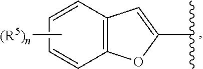

wherein n is 0, 1, 2, 3, or 4.

In some embodiments of Formula II or Formula III, R² is

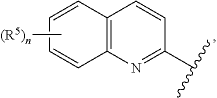

wherein n is 0, 1, 2, 3, or 4.

Representative examples of compounds of the present disclosure include, but are not limited to:

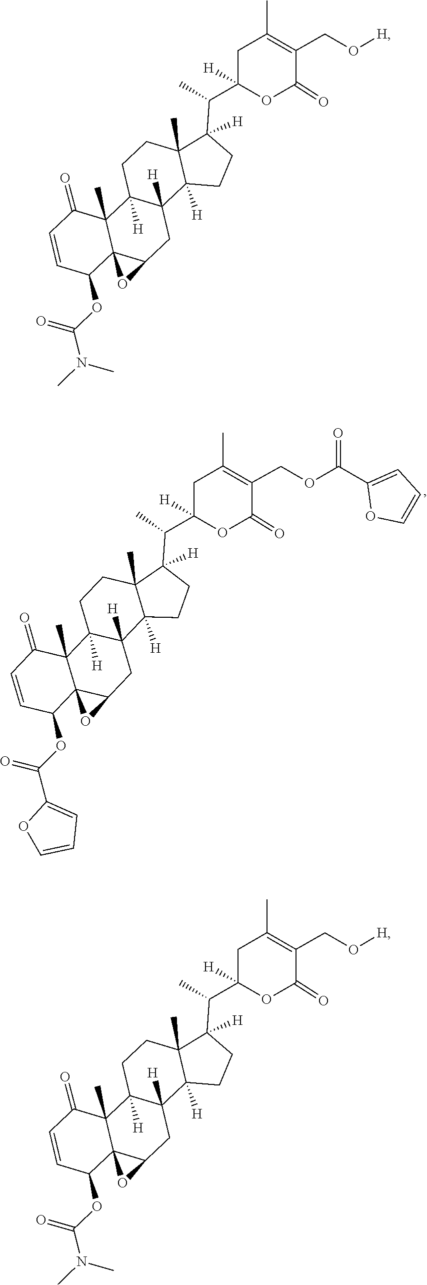

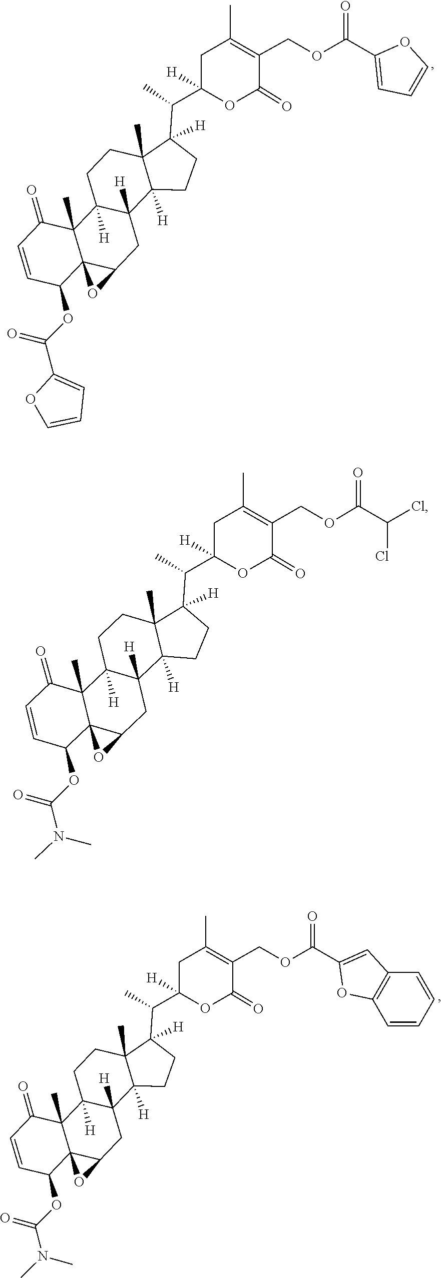

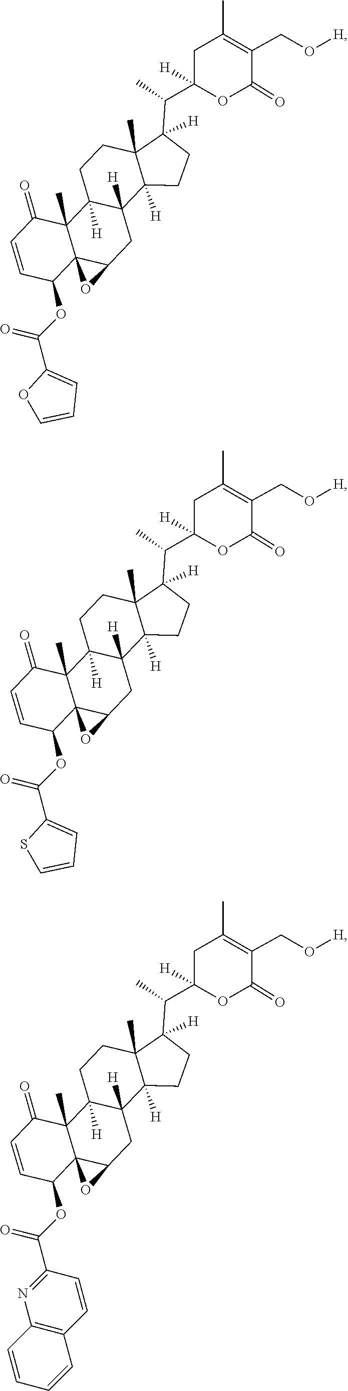

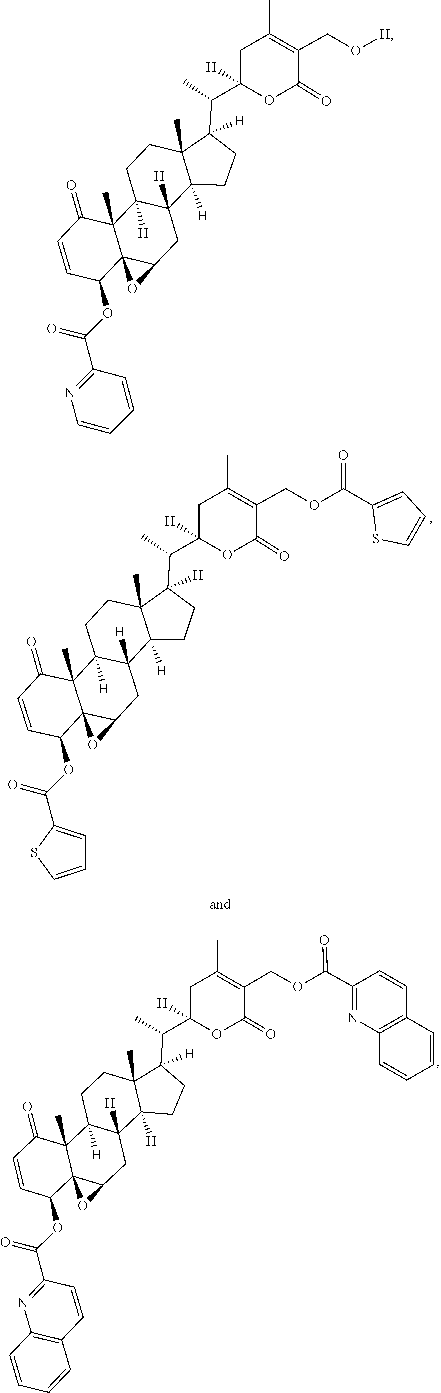

or a pharmaceutically acceptable salt thereof.

“Notch1” refers herein a polypeptide encoded by NOTCH1 gene (Reference No.: HGNC: 7881; Entrez Gene: 4851; Ensembl: ENSG00000148400; OMIM: 190198; UniProtKB: P46531). In some embodiments, the Notch1 polypeptide comprises the sequence of SEQ ID NO: 21, or a polypeptide sequence having at or greater than about 80%, about 85%, about 90%, about 95%, or about 98% homology with SEQ ID NO: 21, or a polypeptide comprising a portion of SEQ ID NO: 21. The Notch1 polypeptide of SEQ ID NO: 21 may represent an immature or preprocessed form of mature Notch1, and accordingly, included herein are mature or processed portions of the Notch1 polypeptide in SEQ ID NO: 21.

It is understood and herein contemplated that the disclosed compounds and pharmaceutical compositions can be used to treat, inhibit, reduce, ameliorate, decrease, and/or prevent any disease or disorder where uncontrolled cellular proliferation occurs such as cancers. Thus, in one aspect are methods of treating, reducing, decreasing, inhibiting, and/or preventing a cancer and/or metastasis in a subject comprising administering to the subject a therapeutically effective amount of any of the compounds or the pharmaceutical compositions disclosed herein.

As used herein more examples of neoplastic disorders and cancers that can be treated using the disclosed methods and/or compositions include but are not limited to lymphoma, PTEN hamartoma syndrome, Familial adenomatous polyposis, Tuberous sclerosis complex, Von Hippel-Lindau disease, ovarian teratomas, meningiomas, osteochondromas, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, breast cancer, and epithelial cancer, renal cancer, genitourinary cancer, lung cancer, esophageal carcinoma, head and neck carcinoma, large bowel cancer, hematopoietic cancers; testicular cancer; colon cancer, rectal cancer, prostatic cancer, and pancreatic cancer. In some embodiments, methods and/or compositions can be applied for treating colorectal cancer, prostate cancer, bladder cancer (e.g., muscle-invasive bladder cancer, or breast cancer (e.g., triple-negative breast cancer).

In some embodiments, the cancer cell or tumor cell has an increased level or activation of Notch1 (e.g., an increased level of phosphorylated Notch1). Accordingly, the methods and/or compositions disclosed herein can decreases a level of Notch1 or phosphorylated Notch 1 in the cell (e.g., a cancer or tumor cell) derived from the subject as compared to a reference level. Thus, in one aspect, disclosed herein are methods of treating, reducing, decreasing, inhibiting, and/or preventing a cancer and/or metastasis in a subject comprising administering to the subject a therapeutically effective amount of any of the compounds or the pharmaceutical compositions disclosed herein; wherein the cancer expresses increased/elevated levels of Notch1 activation.

Given the ability of the compounds disclosed herein to reduce Notch1 signaling and/or activity, it is understood and herein contemplated that the disclosed compositions can be used in any situation where lower levels of Notch1 signaling or activity are desired. Thus, in one aspect, disclosed herein are methods of inhibiting, decreasing, reducing, and/or preventing Notch1 signaling comprising contacting a cell with increased levels of Notch1 signaling and/or activity with any of the compositions disclosed herein. It is further understood that the cell comprising the elevated levels of Notch1 signaling and/or activity can be in a subject and thus the subject in need of decreased levels of Notch1 signaling and/or activity. Accordingly, disclosed herein are methods of inhibiting, decreasing, reducing, and/or preventing Notch1 signaling in a subject in need thereof comprising administering to the subject a therapeutically effective amount of any of the compounds or the pharmaceutical compositions disclosed herein.

Also disclosed herein are method of treating, reducing, decreasing, inhibiting, and/or preventing an infectious disease in a subject comprising administering to the subject a therapeutically effective amount of any of the compounds or the pharmaceutical compositions disclosed herein.

In some embodiments, the infectious disease is caused by infection of a virus, a bacterium, a fungus, or a parasite. Thus, in one aspect, disclosed herein are methods of treating, reducing, decreasing, inhibiting, and/or preventing an infectious disease; wherein the infectious disease is caused by a viral infection, such as, for example, an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERSCoV), Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are methods of treating, reducing, decreasing, inhibiting, and/or preventing an infectious disease; wherein the infectious disease is caused by a bacterial infection, wherein the bacterial infection is an infection with a bacterium selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsia′ species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

In one aspect, disclosed herein are methods of treating, reducing, decreasing, inhibiting, and/or preventing an infectious disease; wherein the infectious disease is caused by a fungal infection, wherein the fungal infection is an infection with a fungus selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

Also disclosed herein are methods of treating, reducing, decreasing, inhibiting, and/or preventing an infectious disease; wherein the infectious disease is caused by a parasitic infection, wherein the parasitic infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

Also disclosed herein are method of treating, reducing, decreasing, inhibiting, and/or preventing a neurological disorder in a subject comprising administering to the subject a therapeutically effective amount of any of the compounds or the pharmaceutical compositions disclosed herein. In some embodiments, the neurological disorder is selected from depression, post-traumatic stress disorder (PTSD), anxiety, and a neurodegenerative disease.

As used herein, the term “neurodegenerative disease” refers to a varied assortment of central nervous system disorders characterized by gradual and progressive loss of neural tissue and/or neural tissue function. A neurodegenerative disease is a class of neurological disorder or disease, and where the neurological disease is characterized by a gradual and progressive loss of neural tissue, and/or altered neurological function, typically reduced neurological function as a result of a gradual and progressive loss of neural tissue. Examples of neurodegenerative diseases include for example, but are not limited to, Alzheimer's disease (AD), Parkinson's disease (PD), Huntington's Disease, Amyotrophic Lateral Sclerosis (ALS, also termed Lou Gehrig's disease) and Multiple Sclerosis (MS), polyglutamine expansion disorders (e.g., HD, dentatorubropallidoluysian atrophy, Kennedy's disease (also referred to as spinobulbar muscular atrophy), spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple system atrophy, Pelizaeus-Merzbacher disease, Pick's disease, primary lateral sclerosis, Refsum's disease, Sandhoff disease, Schilder's disease, spinal cord injury, spinal muscular atrophy (SMA), Steele-Richardson-Olszewski disease, Tabes dorsalis, and the like. In some embodiments, the neurodegenerative disease is Alzheimer's disease. In some embodiments, the neurodegenerative disease is Parkinson's disease (PD). In some embodiments, the neurodegenerative disease is Huntington's Disease.

“Alzheimer's disease” or “AD” as used herein refers to all form of dementia, identified as a degenerative and terminal cognitive disorder. The disease may be static, the result of a unique global brain injury, or progressive, resulting in long-term decline in cognitive function due to damage or disease in the body beyond what might be expected from normal aging. Particularly, it has been identified that Alzheimer's disease is associated with the accumulation of the beta-amyloid protein (or Aβ) and tau, and it has been known that the induction of the degeneration and death of nerve cells caused by the amyloid protein is involved in the mechanism of Alzheimer's disease. The beta-amyloid protein involved in Alzheimer's has several different molecular forms that collect between neurons. One form, beta-amyloid 42, is thought to be especially toxic. Increased levels and/or accumulation of Aβ, tau (e.g., total tau, phospho-tau), or both proteins is a pathological hallmark of AD.

In one example, the methods and/or the compositions disclosed herein can increase the degradation of Aβ protein (e.g., Aβ 42 or Aβ 40) and/or tau protein (e.g., total tau or phosphor-tau) inside a cell or in extracellular fluid of a subject. In one example, the methods and/or the compositions disclosed herein can reduce a level of Aβ protein (e.g., Aβ 42 or Aβ 40) and/or tau protein (e.g., total tau or phosphor-tau) inside a cell or in extracellular fluid of a subject. In one example, the methods and/or the compositions disclosed herein can reduce and/or prevent the accumulation of Aβ protein (e.g., Aβ 42 or Aβ 40) and/or tau protein (e.g., total tau or phosphor-tau) inside a cell or in extracellular fluid of a subject.

It should be understood that a treatment of Alzheimer's disease may be a treatment of one or more of memory loss, poor judgment leading to bad decisions, loss of spontaneity and sense of initiative, repeating questions, having difficulties to organize thoughts, mood and personality changes, and/or increased anxiety and/or aggression. Treatment can be indicated by one or more of mental status and neuropsychological testing indicating improvement in memory, mitigation of memory loss, and/or improvement in other thinking skills, and/or brain imaging (e.g., using magnetic resonance imaging (MRI), computerized tomography (CT), or positron emission tomography (PET)) indicating mitigation of brain shrinkage, amyloid deposits, or neurofibrillary tangles, and/or improvement in nutrient metabolism in brain as compared with prior to treatment of the subject or as compared with the incidence of such symptom in a study population.

Parkinson's disease (PD) refers to a neurodegenerative disorder that is characterized by the loss of dopaminergic neurons and accumulation of Lewy's bodies leading to imbalance in the levels of dopamine. Symptoms of PD include tremors, bradykinesia, muscle stiffness, impaired posture and gait, loss of movement, changes in speech, changes in writing, thinking difficulties, constipation, depression, sleep problems, changes in blood pressure, smell dysfunction and pain and fatigue. The levels of norepinephrine at neuron ends are also affected, leading to non-movement features.

“Depression” or “major depressive disorder” refers to a mood disorder that causes a persistent feeling of sadness and loss of interest. It should be understood that a treatment of depression may be a treatment of one or more of change in depressed mood or loss of interest and pleasure, indifference or apathy, or change in a number of neurovegetative functions (for example, sleep patterns, appetite and body weight, motor agitation or retardation, or fatigue), impairment in concentration and decision making, constant feelings of shame or guilt, and thoughts of death or dying.

“Post-traumatic stress disorder” or “PTSD” refers to a psychiatric disorder that is triggered by a terrifying event, either experiencing it or witnessing it. Treatment of PTSD can be indicated by mitigation of flashbacks, nightmares, anxiety, negative changes in thinking and mood as compared with prior to treatment of the subject or as compared with the incidence of such symptom in a study population.

In some embodiments, the composition described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

As the timing of a cancer can often not be predicted, it should be understood the disclosed methods of treating, inhibiting, reducing, ameliorating, and/or preventing the disease or disorder described herein can be used prior to or following the onset of the disease or disorder, to treat, prevent, inhibit, and/or reduce the disease or disorder or symptoms thereof. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of the disease or disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the disease or disorder.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1: ASR488, a Novel Small Molecule, Activates an mRNA Binding Protein, CPEB1, and Inhibits the Growth of Bladder Cancer

### Introduction

Bladder cancer (BCa) is one of the major cause of cancer related morbidity in the US and worldwide. In 2019, around 80,270 new cases of BCa were expected, and 17,670 estimated BCa patient deaths in the US. Most cases of BCa are non-muscle invasive and transurethral resection is commonly performed but with a high recurrence rate. However, nearly 25% of newly diagnosed BCa patients have muscle invasive bladder cancer (MIBC), and approximately half of the patients with MIBC recurrence eventually die from BCa because of the lack of treatment options. Radical cystectomy followed with pelvic lymphadenectomy is the gold standard treatment for MIBC. Bladder preserving trimodal therapies (TMT) are also evolving as an effective alternate, however, combined with current platinum-based chemotherapy (such as MVAC-methotrexate, vinblastine, adriamycin, and cisplatin), 25-30% patients still require salvage cystectomy. The high morbidity of definitive therapy for BCa along with poor prognosis of advanced BCa warrants identification of novel targets and subsequent therapeutic interventions to achieve complete remission of BCa.

To overcome the disadvantages of traditional chemotherapy and radiotherapy, the research paradigm has shifted towards elimination of cancer cells specifically by targeting specific molecular targets. To achieve this for use in clinical practice, the current preferred approaches are search for novel and targeted small-molecule agents and monoclonal antibodies (mAbs). mAbs are usually large molecular weight proteins (˜150 kDa), whereas small molecule cancer drugs can transfer through the plasma membranes owing to their much smaller in size (≤500 Da). The cost-effectiveness and their amenable nature to oral administration make them a better choice than mAbs, which are mostly administered intravenously.

Small molecules are being extensively used to target oncogenic pathways that are aberrantly activated in most cancers. These molecules function by targeting the kinases that include receptors as well as their downstream regulators thus inhibiting cancer cell survival and proliferation. A better understanding of oncogenic mechanisms, as well as identification of specific genes/proteins can help design novel strategies to not only improve the efficacy of current drugs but can also be a major aid in the identification of novel agents. Advancement of systems biology can help us analyze and identify interactions between important gene networks that can provide significant insight into biological pathways. The genetic processes are very complex, and these interactions change significantly when a cancer cell is treated with a small molecule.

Based on the structure-activity relationship (SAR) studies focused on the Withaferin A (a dietary compound which exhibits anti-cancer effect against many cancer types) analogs, a novel small molecule, ASR488, was designed by protecting —OH group at 4-position of Withaferin A by thiophene-2-carbonyl functionality. ASR-488 demonstrated cell growth arrest in BCa cells and, more importantly, is non-toxic to normal BCa cells. In the current study, differential gene network analysis was performed to detect the changes in gene expression in ASR488 treated MIBC cells. Also, functional annotation and network analyses were performed to identify differential gene expression (DEGs). Analysis of the biological functions and networks of ASR488-treated MIBC cells helps gain a better understanding of the effect of small molecules and to explore the candidate BCa treatments.

Materials and Methods

Synthesis of ASR488. ASR488 was synthesized starting from Withaferin A according to a synthetic strategy. Briefly, to a mixture of Withaferin A and trimethylamine in methylene chloride at 0° C. was added 2-thiophenecarbonyl chloride and the resulting reaction mixture was stirred overnight at room temperature. The reaction mixture was quenched with saturated NaHCO₃ solution, extracted with methylene chloride, and purified by column chromatography. The compound was characterized by NMR and MS and its purity (>98%) was determined by HPLC.

Cell culture and viability assay. BCa cell lines TCCSUP (ATCC® HTB5™), and HT1376 (ATCC® CRL-1472™) were purchased from ATCC (American type culture collection; Manassas, Va., USA). Cell lines were maintained in Eagle's Minimum Essential Medium at 37° C. and 5% CO₂. The anti-proliferative effect of ASR488 was determined by the MTT (3-[4, 5-dimethylthiazol-2-yl]-2, 5-diphenyltetrazolium bromide) assay. TCCSUP and HT1376 cells were treated with varying concentrations of ASR488 (0.2-12.5 μM) for 24, 48, and 72 h.

Detection of apoptosis by flow cytometry and immunoblotting. Annexin V-fluorescein isothiocyanate (FITC) against propidium iodide (PI) assay (FITC Annexin V Apoptosis Detection Kit I, BD Pharminogen, San Diego, Calif., USA) was used for detecting apoptosis as described previously. Total protein extracts from TCCSUP cells were prepared with the Mammalian Protein Extraction Reagent (Thermo Scientific, Rockford, Ill., USA) according to the manufacturer's instructions. Western blotting was performed using specific antibodies against Cleaved PARP (Cat #5625), BAX (Cat #5023) (Cell signaling Technology), and β-actin (Santa Cruz Biotechnologies, Dallas, Tex., USA). The positive bands were detected using enhanced chemiluminescence.

RNA isolation, cDNA library construction, and DNA sequencing. TCCSUP cells treated with vehicle (DMSO) or ASR488 were subjected to RNA isolation using TRIzol® (Invitrogen; Thermo Fisher Scientific, Inc., Waltham, Mass., USA) according to the manufacturer's protocol. Nano Photometer® spectrophotometer (Implen, Inc., Westlake Village, Calif., USA) was used to measure the RNA concentration and purity of the samples. A cDNA library was then constructed using an NEB Next® Ultra™ RNA Library Prep kit for Illumina® (New England Biolabs, Inc., Ipswich, Mass., USA) by Novogene Bioinformatics Technologies Co. Ltd. (Beijing, China) following the manufacturer's protocols. Subsequently, polymerase chain reaction (PCR) was, Universal PCR primers, and Index (X) Primer for double-stranded cDNA amplification. The PCR products generated for PCR performed using Phusion High Fidelity DNA polymerase were purified using the AMPure XP system, and Agilent Bioanalyzer 2100 system was used to analyze the library quality. The cDNA library was sequenced using an Illumina Hiseq 2000/2500 platform and 100 bp/50 bp single-end reads were generated.

Data Analysis. Bioinformatics analysis was performed using a combination of programs including STAR, HTseq, Cufflink, and our wrapped scripts. Tophat program was used to parse the alignments and DESeq2/edgeR was utilized to ascertain differential expressions. To determine GO and KEGG enrichment Cluster Profiler was used.

Clustering. Fragments Per Kilobase of transcript per Million mapped reads (FPKM) expression level was used to cluster different samples to identify the correlation between differences. To analyze the difference between DEGs and the correlation, hierarchical clustering distance method was used with default parameters in R with the function of the heatmap, SOM (Self-organization mapping), and kmeans using silhouette coefficient.

GO and KEGG enrichment analysis of differentially expressed genes. Cluster profiler R package with corrected gene length bias was used for analyzing GO enrichment of DEGs. P<0.05 were considered significantly enriched by DEGs. Cluster profiler R package was also used to examine the statistical enrichment of differential expression genes in KEGG pathways for understanding functions as well as molecular level information of the dataset generated by the RNASeq.

Differential Expression Analysis

For DESeq2 with biological replicates: DESeq2 R package (2_1.6.3) was used to analyze differential expression between the ASR488 treated and control groups (two biological replicates per condition). The p-values obtained from the analysis were adjusted using the Benjamini and Hochberg's approach so that the false discovery rate (FDR) can be controlled. The genes having adjusted p-value <0.05 were assigned as differentially expressed.

For edge R without biological replicates: EdgeR program package (3.16.5) was used to adjust the read counts for each sequenced library before differential gene expression analysis, through one scaling normalized factor. The p-values were adjusted using the Benjamini and Hochberg method. A corrected p-value of 0.05 and absolute fold change of 1 were set as the thresholds for significantly differential expression. The Venn diagrams were prepared using the function Venn diagram in R based on the gene list for different groups.

ASR488 Treatment Inhibits MIBC Cell Growth.

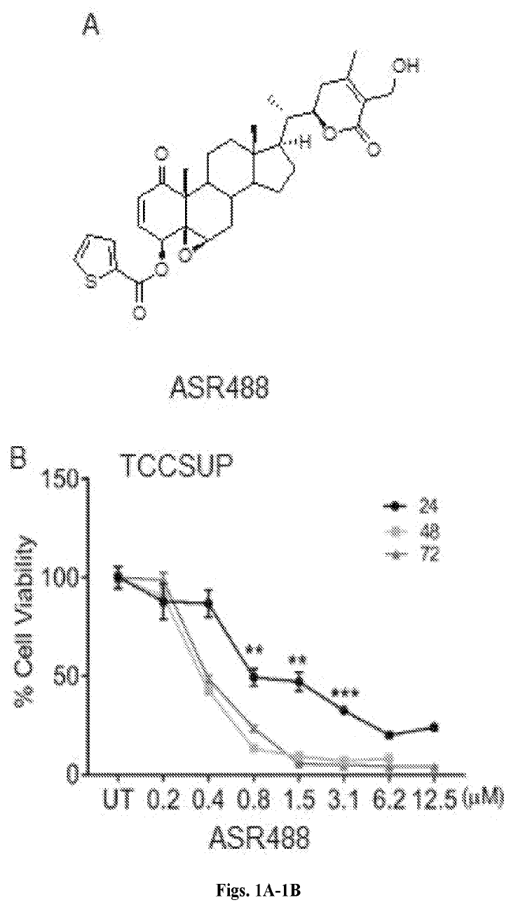

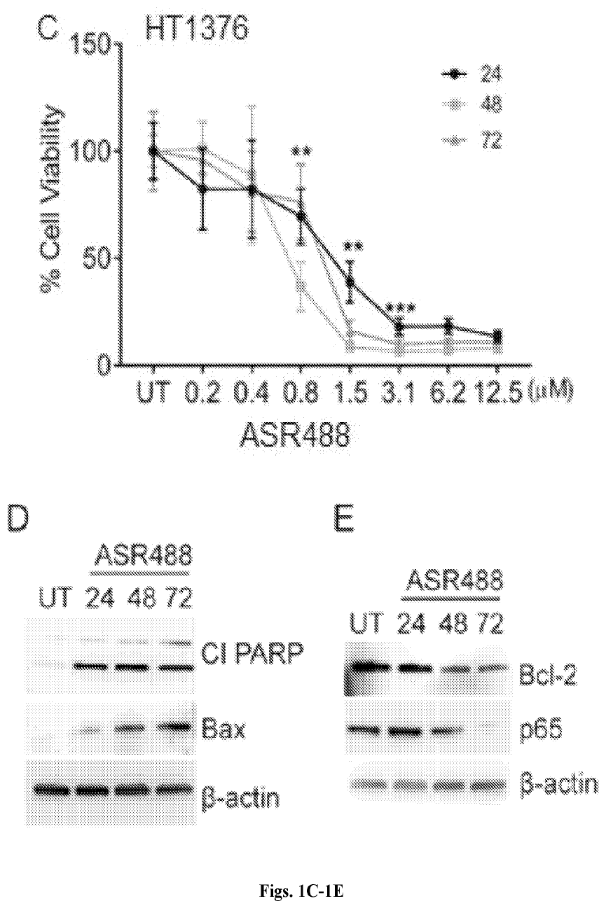

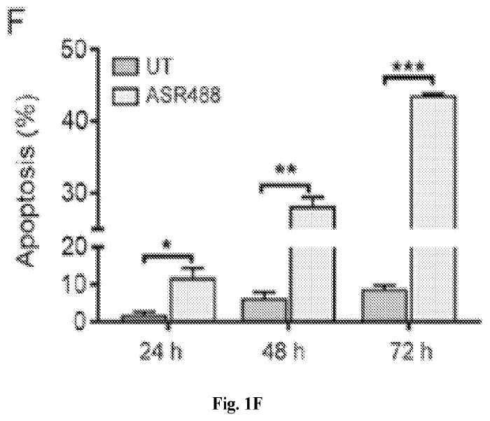

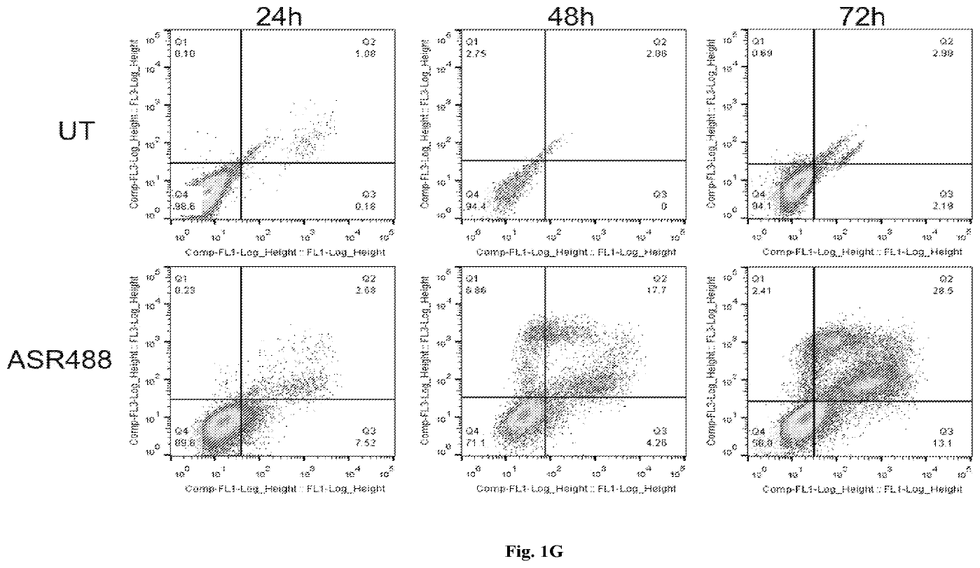

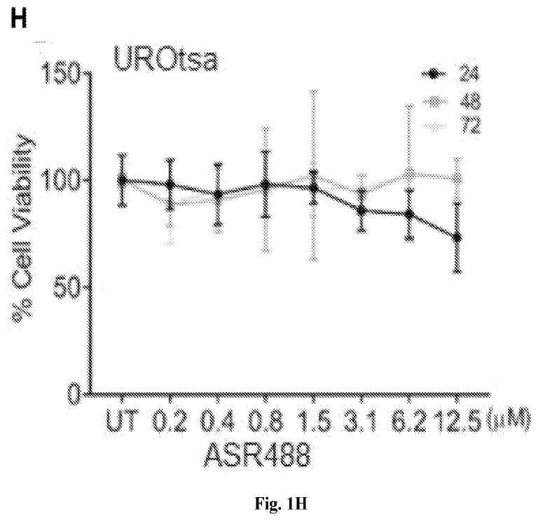

To determine the therapeutic potency of ASR488 (FIG. 1A) on MIBC, the effect of ASR488 treatment on cell viability of TCCSUP and HT1376 cells was examined using the MTT assay. Significant reductions in cell viability were observed in both TCCSUP (IC₅₀ at 800 nM, 480 nM, and 450 nM at 24, 48 and 72 h, respectively) and HT1376 (IC50 at 1.28 μM, 750 nM, and 850 nM at 24, 48, and 72 h, respectively) cell lines (FIGS. 1B, 1C). Induction of apoptosis can be interpreted by observation of increased expression of BAX and Cleaved PARP (FIG. 1D). ASR488 treatment inhibited survival signaling such as downregulation of p65 and Bcl-2 expression in ASR488 treated MIBC cell lines (FIG. 1E). Annexin V-FITC staining further corroborated the results by showing significant increases in apoptosis in both cell lines (TCCSUP: 30.5%, p=0.0382 and HT1376: 23.2%, p=0.0131) after 24 h treatment of ASR488 (FIGS. 1F, 1G). Overall, these results indicate that ASR488 effectively initiated apoptotic signaling, which resulted in significant growth inhibition of MIBC cells.

Identification of Differentially Expressed Genes in ASR488-Treated TCCSUP Cells

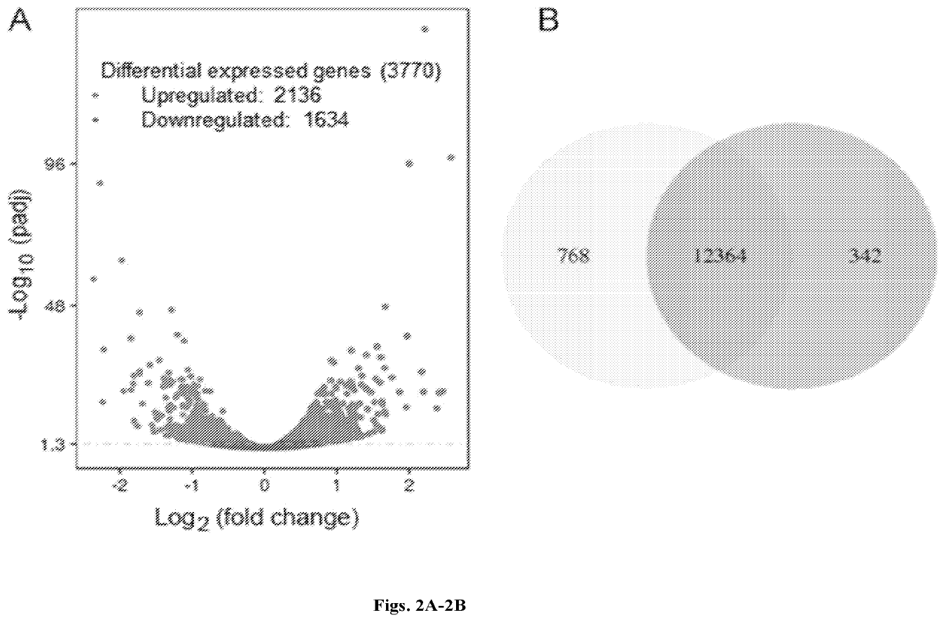

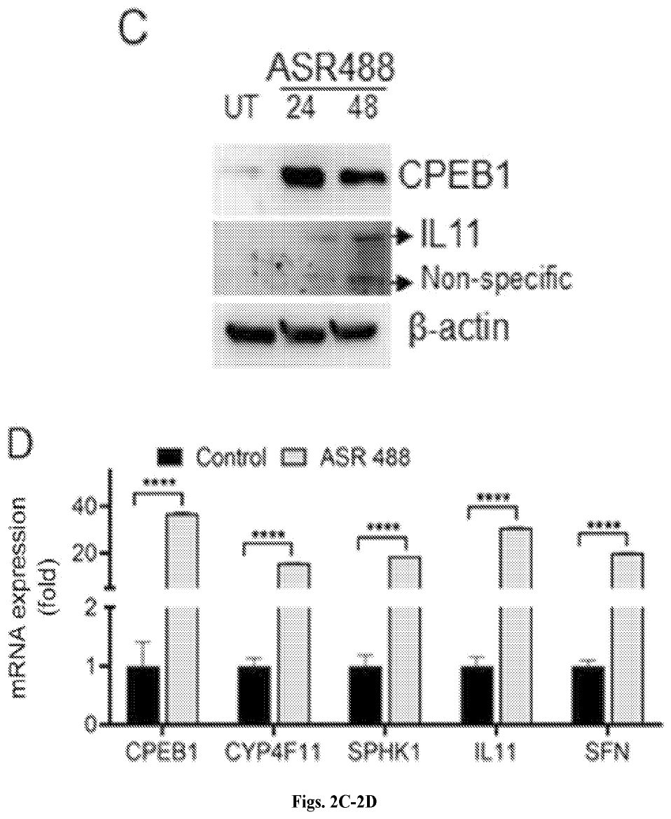

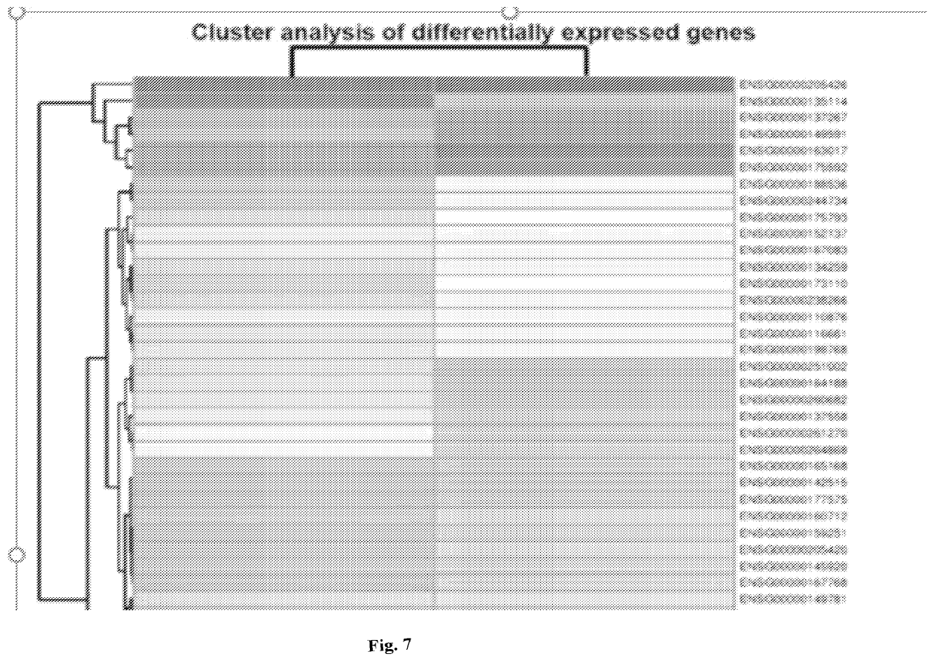

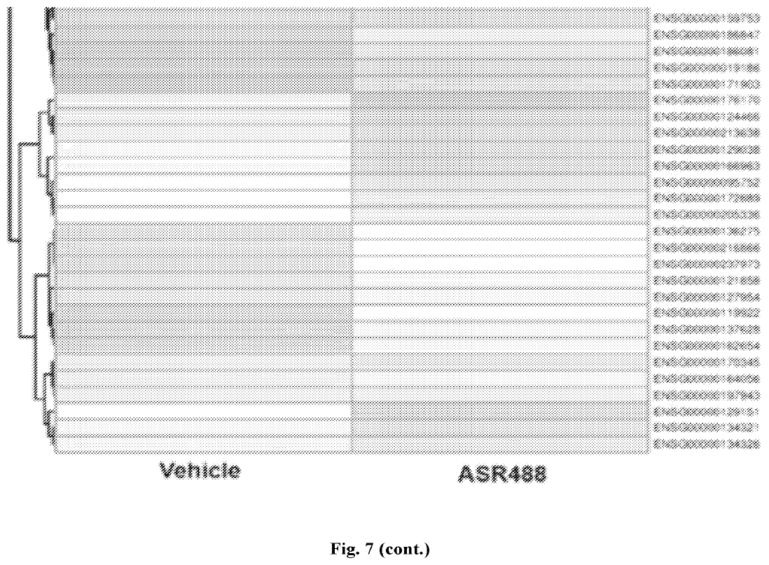

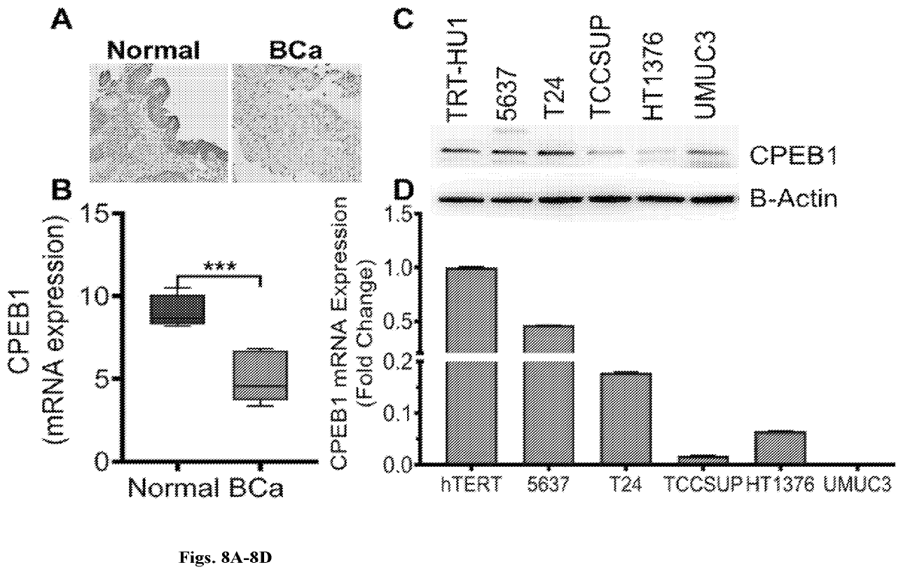

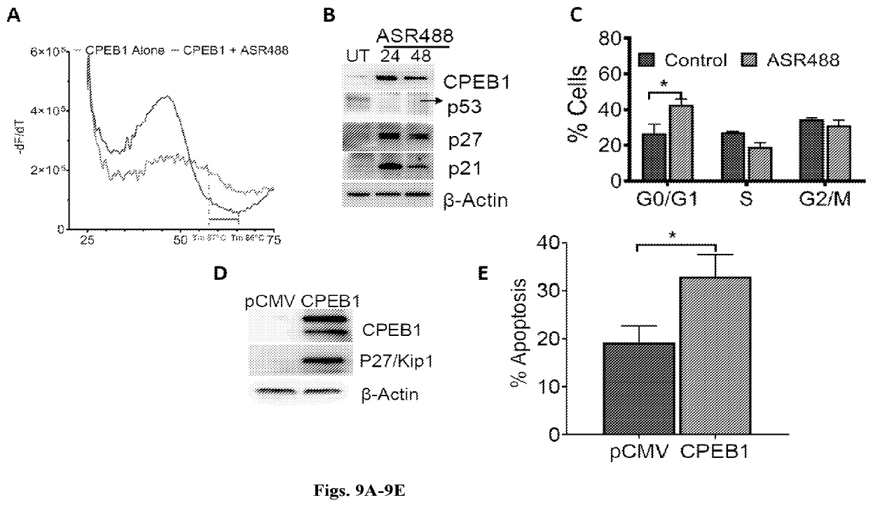

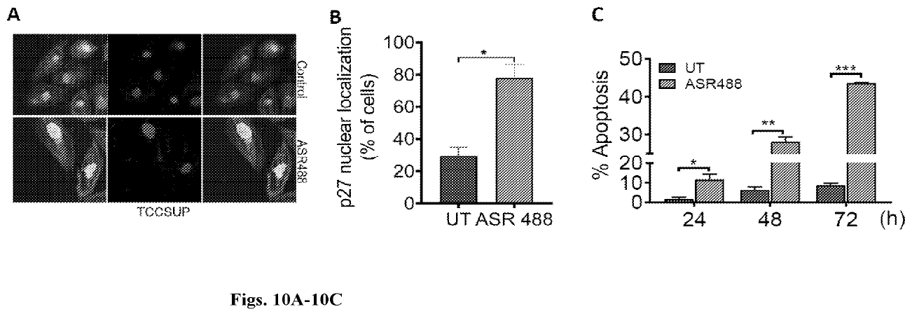

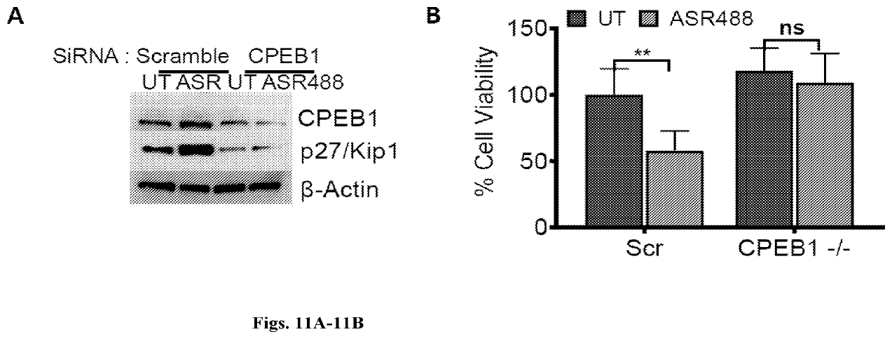

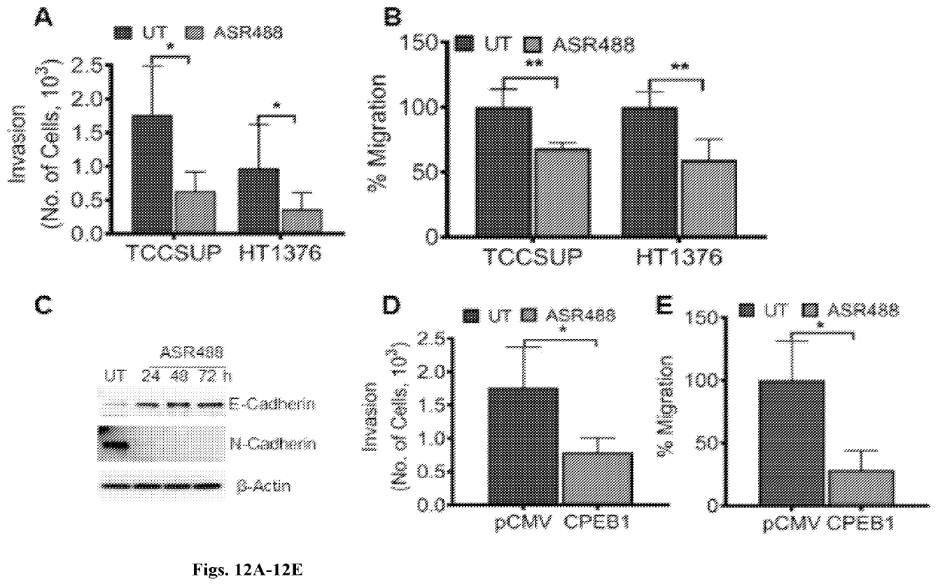

To identify whether there is a significant change in expression of key regulatory genes in ASR488 treated cells, RNASeq data for DEGs were performed and analyzed in ASR488-treated and vehicle-treated TCCSUP cells. A volcano map demonstrated the overall distribution of DEGs between control and ASR488-treated BCa cells. The genes presented as red dots are the upregulated genes, whereas the downregulated genes are represented as green dots, and blue dots represent the genes that remained unchanged (FIG. 2A). The current study obtained 3770 genes that were differentially expressed in TCCSUP cells upon ASR488 treatment, of which 2136 genes were upregulated, and 1634 were downregulated (FIG. 2A). Lists of the ten most upregulated and downregulated genes in ASR488-treated MIBC cells are given in Tables 1 and 2. Specifically, expression levels of CPEB1, ACTG2, SFN, HSPA6, CYP4F11, TAGLN, LINC00707, IL11, MAP1A, SPHK1, and GNGT2 were upregulated in treated TCCSUP cells, whereas expression levels of SFRP4, DDX60, GBP4, BBOX1, RSAD2, OASL, FOS, IFIT2, CMPK2, STEAP4, and IFI44L were the downregulated. The top five upregulated genes were confirmed by qRT-PCR analysis: CPEB1 (36-fold), IL11 (30-fold), SFN (20.12-fold) and CYP4F11 (15.8-fold) (FIG. 2D, primer details: Table 3), while no significant change was observed in downregulated genes. The top two upregulated genes CPEB1 and IL-11 expressions were confirmed by immunoblotting (FIG. 2C). To identify significant DEGs during ASR488 treatment, the expression quantity of each gene in untreated and ASR488-treated TCCSUP cells was also compared pairwise and filtered with [log 2(fold change)] >1 and q value <0.005. 13,474 DEGs were detected in both datasets (FIG. 2B). Among these, 12,364 genes showed significantly differential expression in both groups. Three-hundred-forty-two genes in the ASR488 treated cells and 768 genes in the control cells showed significantly differential expression (FIG. 2B). To visualize the similarities between the two groups and also to determine if the expression profile of ASR488-treated TCCSUP cells and control cells are different, the genes that were differentially expressed in pairwise comparison were clustered. The dendrogram showed that the gene profile from vehicle-treated BCa cells was distant from that of ASR488-treated TCCSUP cells (FIG. 7). These results confirm that treating metastatic BCa cells with ASR488 leads to differential expression of key genes.

Functional Enrichment of DEGs Via GO

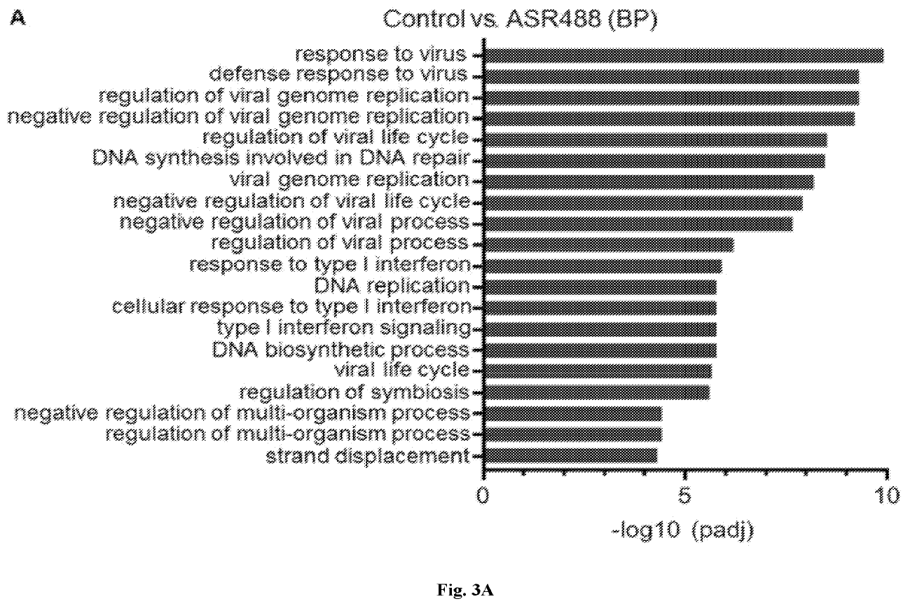

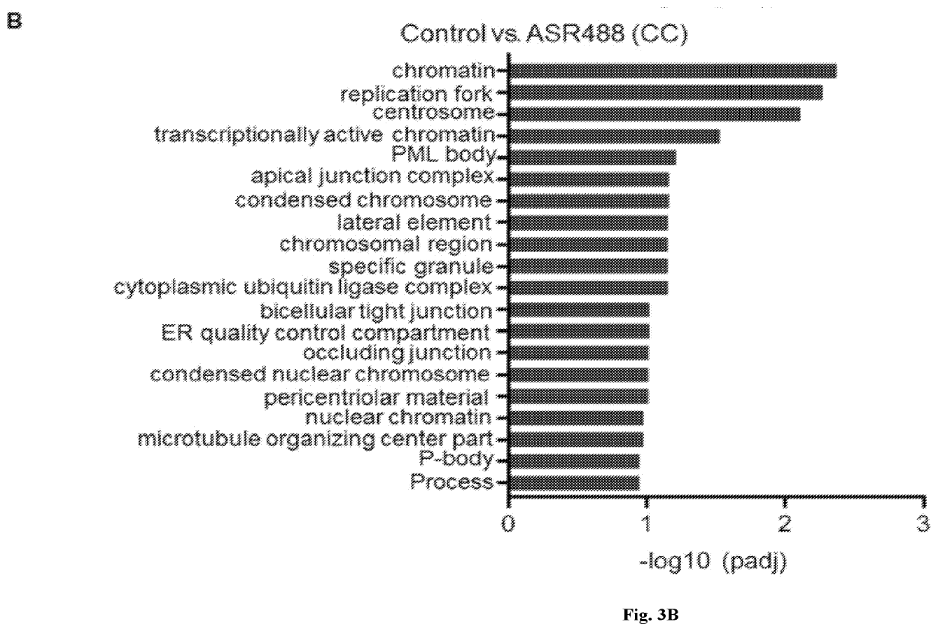

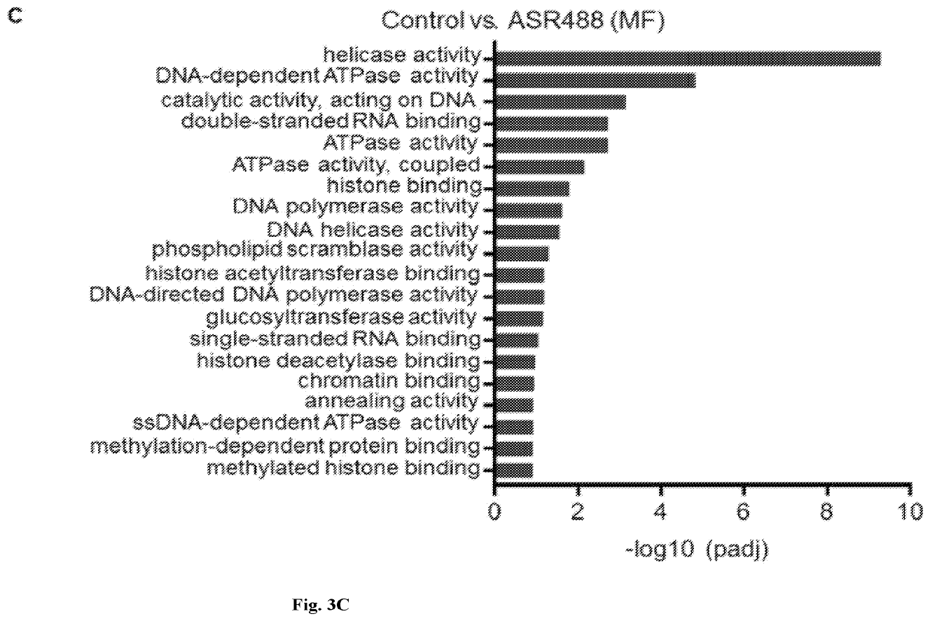

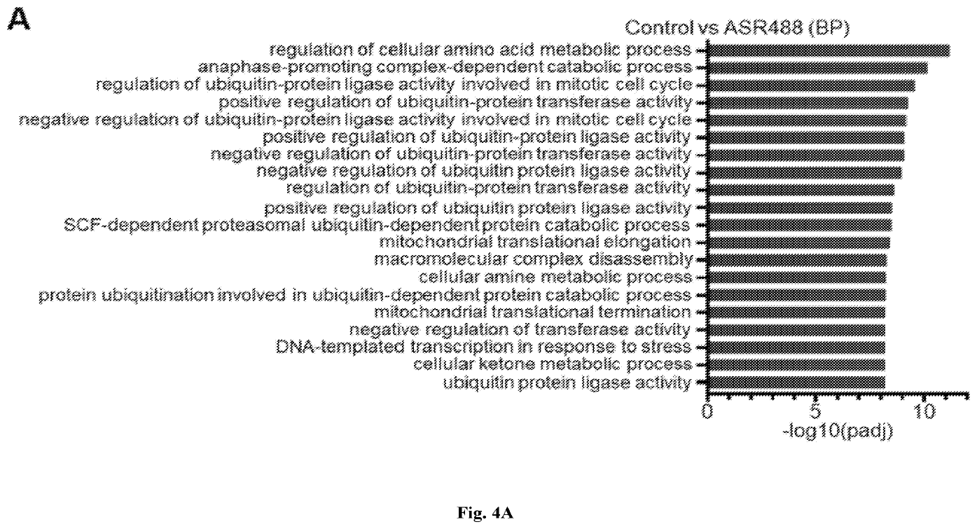

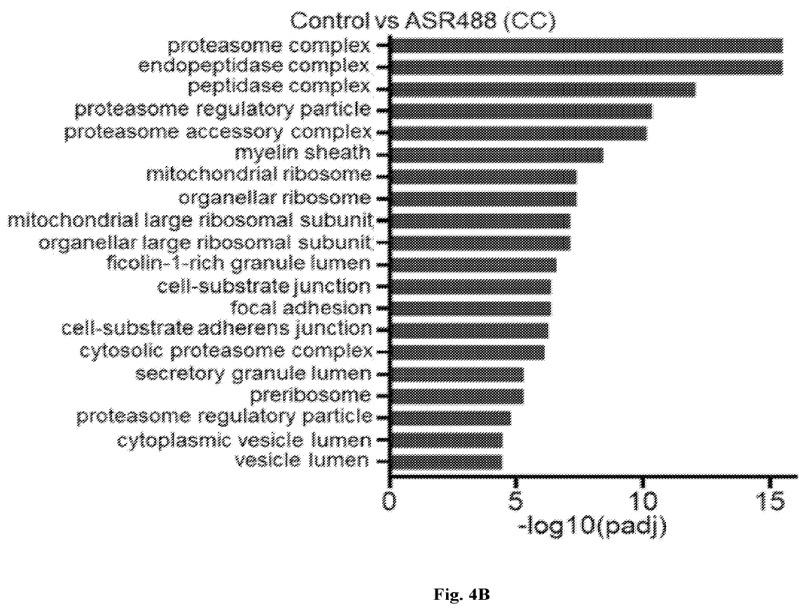

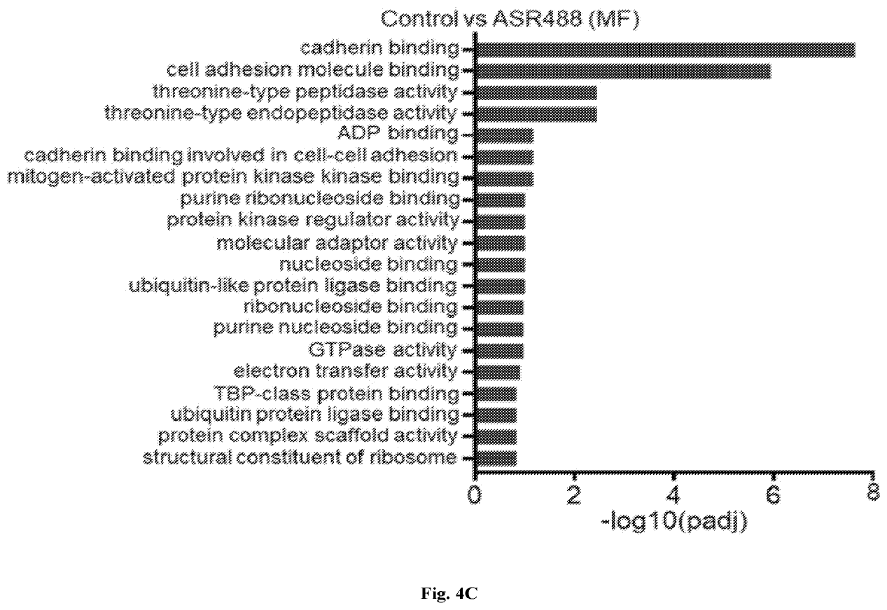

To further visualize the relationship between genes in ASR488-treated TCCSUP in context of their expression, distinct clusters of genes were extracted and submitted to gene set enrichment analysis. The GO terms as well as pathways that were significantly over-represented among genes were identified from the clusters. The top GO terms (from BP (Biological Process), CC (Cellular Component), and MF (Molecular Function) categories) enriched by the upregulated and downregulated DEGs were identified (FIGS. 3 and 4). The results revealed that the downregulated genes were involved in viral defense response, DNA synthesis and repair (BP category, FIG. 3A), transcriptionally active chromatin, endoplasmic reticulum quality (CC Category, FIG. 3B), kinase activity, and DNA polymerase activity (MF category, FIG. 3C). On the other hand, the upregulated genes were mainly associated with regulation of cellular metabolic processes and regulation of ubiquitin-protein ligase activity in the BP category (FIG. 4A). In the CC category, these downregulated genes were involved in the regulation of the proteasome complex, endopeptidase complex, and myelin sheath (FIG. 4B). Whereas, in the MF category, these were mainly involved in cadherin binding, cell molecular adhesion binding, and threonine-type endopeptidase activity (FIG. 4C).

KEGG Pathway Analysis of DEGs.

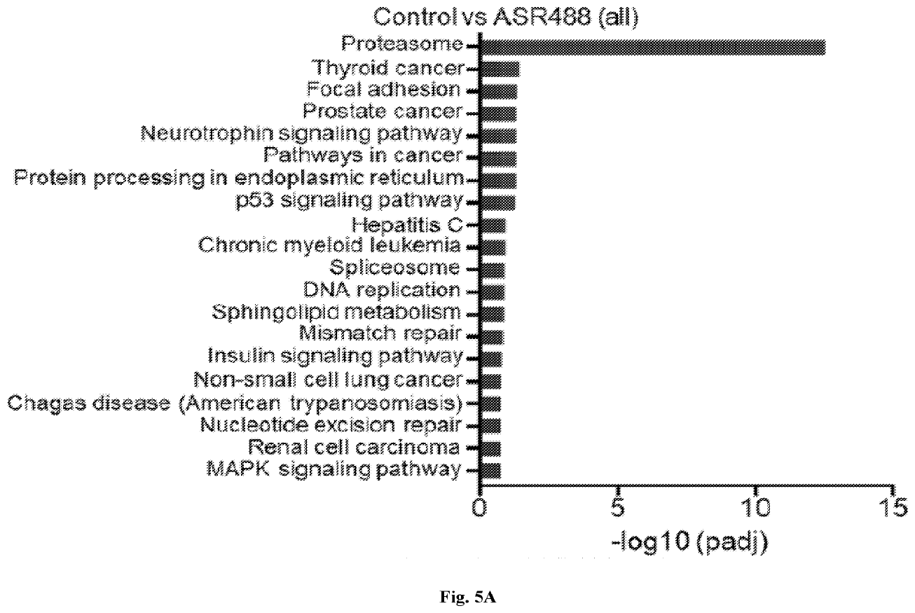

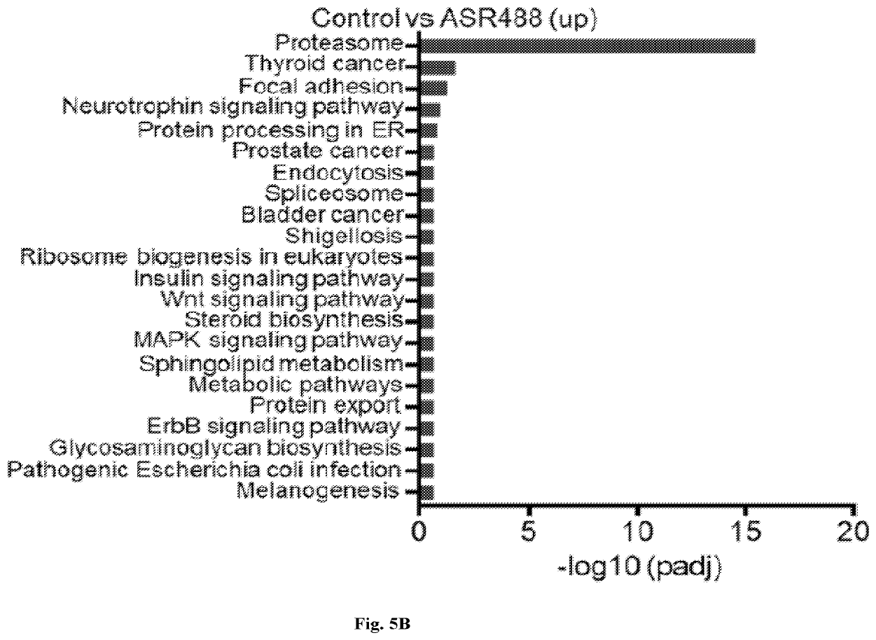

To analyze the functional status of DEGs i.e., which DEGs are activated and suppressed in different classes of pathways, the information we got from gene expression analysis of ASR488-treated TCCSUP cells was mapped to the KEGG pathway. Pathway analysis and functional annotation for up- and down-regulated genes were performed. The analysis revealed that 156 up-regulated genes (padj<0.001, log 2 FC>2) and 82 down-regulated genes (padj<0.001, log 2 FC<−2) were mapped to 238 KEGG pathways. The top 20 enriched pathways are displayed in FIG. 5. The results indicate that the DEGs are highly clustered in several signaling pathways, such as focal adhesion, neurotrophin-signaling, and p53 signaling, as well as in protein processing in the endoplasmic reticulum and BCa (FIG. 5B). The down-regulated pathways in ASR488-treated BCa cells were enriched in DEGs involved in DNA replication, mismatch repair, RNA degradation, nucleotide excision repair, TGFβ signaling, and pathways in cancer (FIG. 5C). Downregulation of the DNA replication, mismatch repair, and pathways in cancer make the ASR488 treated TCCSUP cells less proliferative and invasive, finally contributing to the decreased tumorigenic capacity of the cells.

Reactome Pathway Analysis of DEGs.

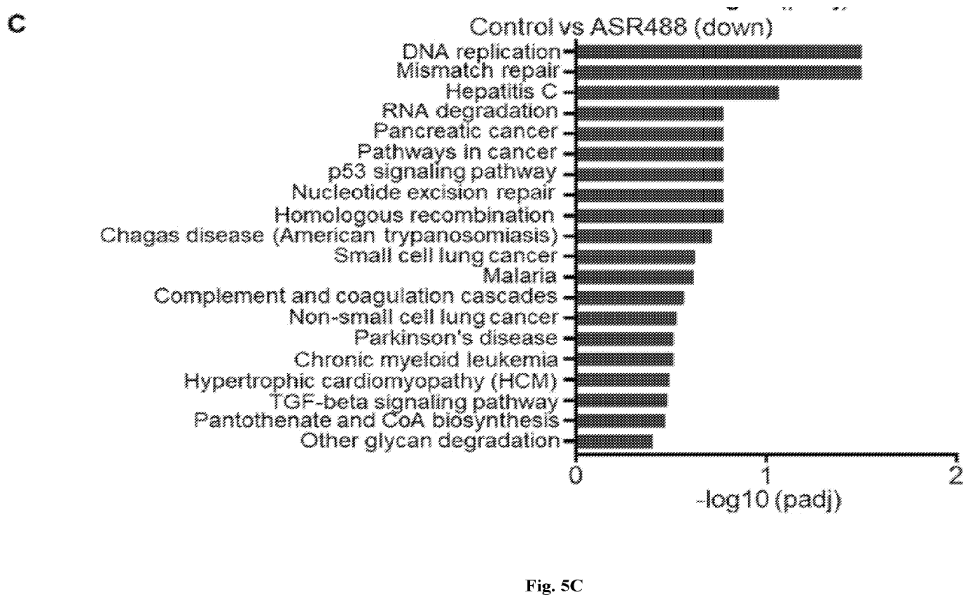

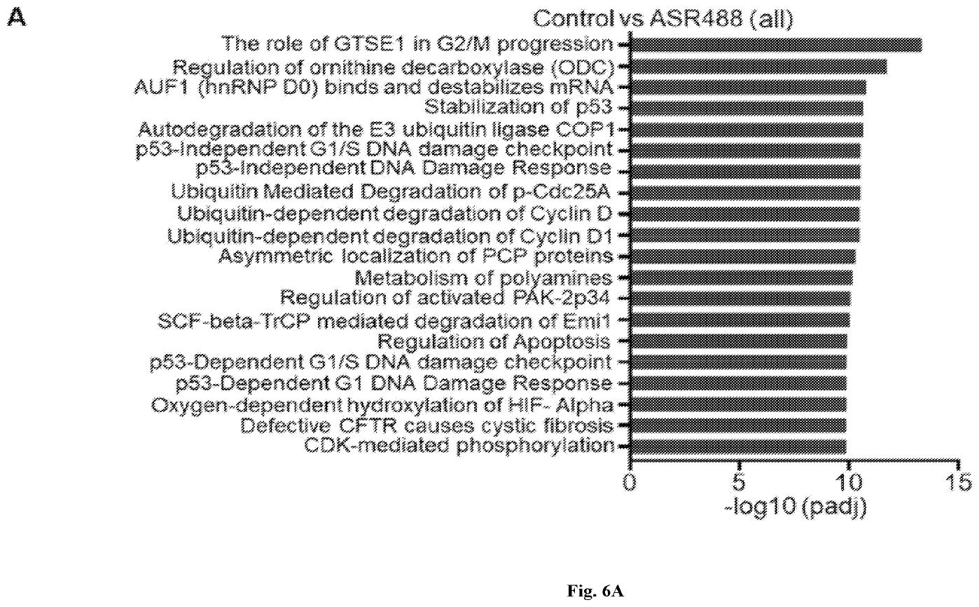

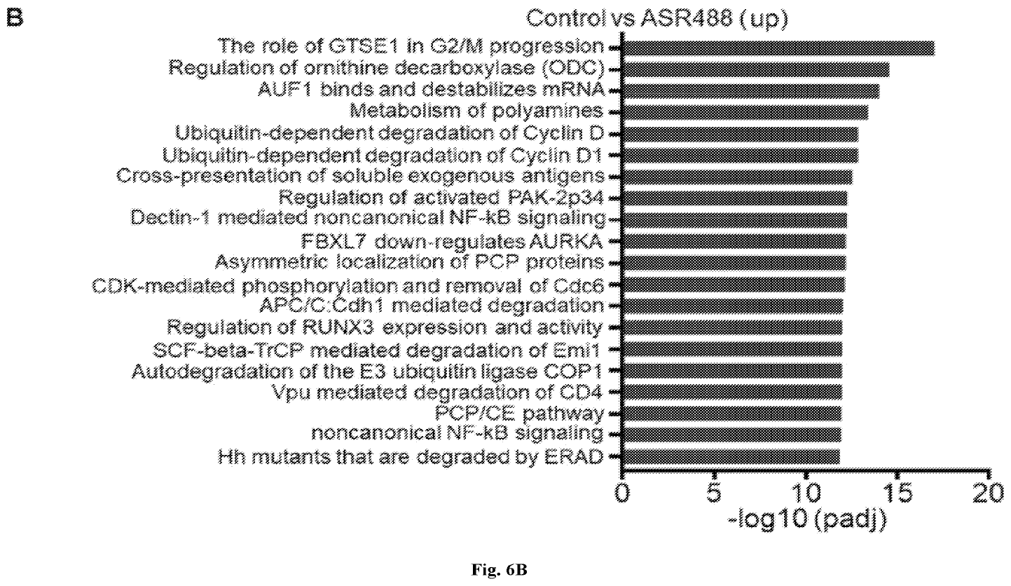

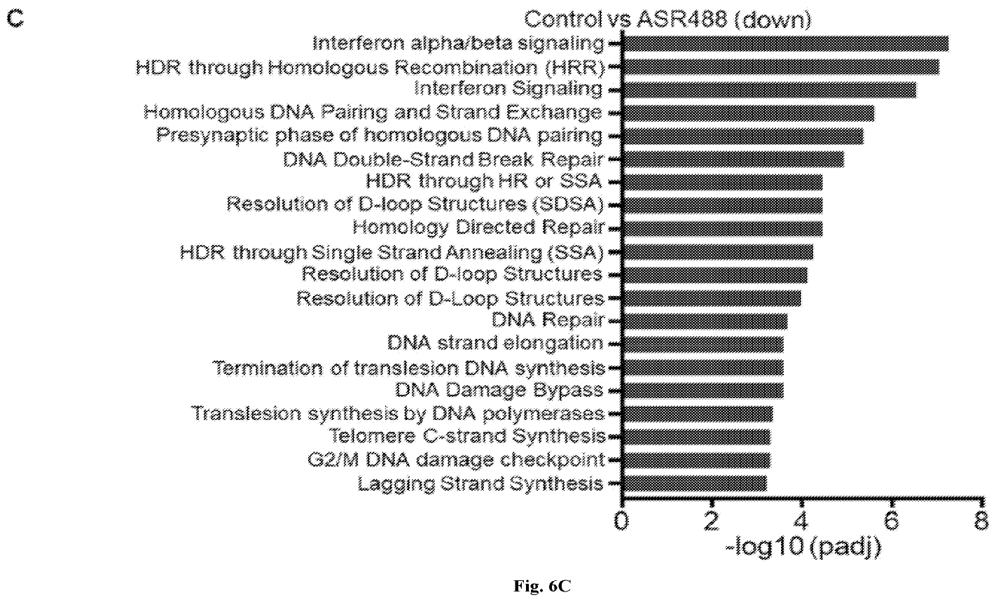

To further analyze gene sets (pre-defined groups of genes that are functionally related), a reactome enrichment analysis was performed (FIG. 6A). It is well established that consistent perturbations over such gene sets frequently cause mechanistic changes. The results demonstrate that the significantly enriched reactome pathways of upregulated DEGs were related to ornithine decarboxylase regulation, regulation of tumor suppressor RUNX3 expression, and non-canonical NF\acute{κ}B signaling (FIG. 6B). The reactome data indicated that gene sets related to ubiquitin-dependent degradation of cyclin D1 were significantly upregulated, which indicated arrest of the cell cycle in the treatment group and supported the growth inhibitory effect of ASR488 treatment in TCCSUP cells (FIG. 6B). The data also demonstrate significant downregulation of gene networks involved in telomere C strand synthesis and DNA damage checkpoints (FIG. 6C).

Among the limited options available to patients with BCa, programmed cell death protein 1 (PD-1) pathway inhibitors are a major category of inhibitors. However, a minority of patients respond, and options after disease progression are a significant unmet need. Over 20 small molecule drugs are being successfully used in cancer treatment after being approved for clinical use. Nevertheless, these are not without limitations. Non-specific binding to multiple molecular targets such as cell surface receptors increase the risk of toxicity. It is thus important to screen the promising small molecules for their effect on crucial pathways, which can remain largely unaffected during treatment of BCa. Analysis of complex signaling networks and genes, which are differentially expressed after treatment, can provide valuable input before progressing to further preclinical as well as clinical trials.

A library of small molecules (analogs of Withaferin A) were screened and significant growth inhibition and induction of apoptosis was observed in MIBC cells with ASR488 treatment. To further explore the mechanism of action of ASR488 in regulating the growth of BCa, the gene expression profiling was analyzed using RNA-seq.

ASR488 treatment significantly affected the expression of key regulatory genes, such as CPEB and IL-11. Depletion of CPEB1 expression levels has been explicitly linked with increased metastatic potential in different cancer types. CPEB1/2 downregulate TWIST1 expression, which is considered one of the main inducers of EMT. It is also been shown that skin and lung cells were able to circumvent the M1 crisis stage of senescence in CPEB knockdown cells by undergoing telomere erosion, and its reintroduction restored the senescence-like phenotype. The knockdown was also followed with recommencement of cellular growth and fewer mitochondria. These cells had reduced respiration and reactive oxygen species (ROS) and resembled transformed cells by having normal ATP levels, and enhanced rates of glycolysis. Mitochondrial translational elongation and telomere C strand synthesis was significantly affected in GO and reactome enrichment analysis, respectively. Additionally, CPEB knockdown cells have p53 mRNA with an unusually short poly(A) tail, which ultimately resulted in a significant decrease (greater than 50%) in p53 protein levels. The reactome analysis herein indicates that ASR488 treatment significantly affected p53 stabilization and the p53 dependent DNA damage checkpoint. Overall, these findings indicate that regulation of mitochondrial processes and p53 stabilization can be mechanisms for cell growth arrest in ASR488-treated BCa cells.

Another significantly upregulated gene in our study, IL-11, has been shown to be dysregulated in human gastric, colon, breast, and bladder cancers. Unlike IL-6, the role of IL-11 in various inflammation-associated cancers is not well studied. Interestingly, IL-11 has generally been considered as an anti-inflammatory cytokine, which is in contrast with the well-studied proinflammatory function of IL-6. Although aggressiveness of several cancer types has been attributed to increased IL-11 levels, a decrease in IL-11 has been specifically recognized as a factor contributing to carcinogenesis of the bladder. The expression of IL-11 was downregulated in human BCa cell lines and transitional cell carcinoma (TCC) when it was compared with primary human bladder cell culture. The same study also demonstrated that the BCa patients samples had reduced urinary levels of IL-11 in comparison to healthy subjects. In the present study, another important signaling immune pathway (the TGFβ pathway) was significantly downregulated in KEGG analysis. It has been demonstrated that levels of EMT markers, such as vimentin, slug, and twist, are downregulated in TGFβ knockout mice, and abrogation of TGFβ pathway depletes tumorigenic and invasive potential in an induced mouse BCa model. As discussed in an earlier section, there is also a proven direct link between CPEB expression and downregulation of twist1, CPEB overexpression combined with downregulation of TGFβ signaling during ASR488 treatment can reduce the metastatic potential of BCa cells.

Another observation from the GO enrichment analysis was the significant downregulation of ATPase activity in ASR488-treated BCa cells. ATPase is considered as an important ion transporter that is involved in signal transduction. It is well established that ATPase expression profile is altered in various tumors, such as breast cancer. Inhibition of ATPase activity significantly reduced cell proliferation, motility, and invasion in breast cancer. More recently, downregulation of longevity assurance homolog 2 of yeast LAG1 (LASS2) has been associated with a poor prognosis in patients with BCa. LASS2 binds directly to subunit C of vacuolar H+-ATPase (V-ATPase) and its silencing resulted in increased ATPase activity, which, in turn activated secreted matrix metalloproteinase (MMP)-2 and MMP-9, and thus enhanced cell proliferation, cell survival, and cell invasion in vitro, as well as increase of BCa growth rate in vivo. This decrease in ATP activity is important to point out as CPEB knockout results in resumption of cell growth, fewer mitochondria, and resembled transformed cells by maintaining normal ATP levels by increasing glycolysis. Hence, with normal ATPase activity or levels, the cells can bypass the M1 crisis stage of senescence and thus act as transformed cells with increased proliferative profile. However, an increased CPEB level and decrease in ATPase activity can be detrimental to the growth of these cancer cells and, ultimately, lead to downregulation of metastatic and proliferative capacities.

In summary, using RNAseq data identifies signaling molecules and pathways that are significantly affected upon ASR488 treatment in MIBC cells. These pathways are interlinked in a way that reduces the proliferative and metastatic efficacy of MIBC cells. This study also indicated that ASR488 can be a small molecule for BCa treatment.

TABLE 1
List of top 10 upregulated genes in ASR488-treated TCCSUP cells.

| Gene symbol | Log₂ (fold-change) | P-value |
|---|---|---|
| CPEB1 | 6.3347 | 4.17 × 10⁻³ |
| CYP4F11 | 8.3568 | 1.09 × 10⁻⁸ |
| SPHK1 | 4.6389 | 1.32 × 10⁻⁴¹ |
| IL11 | 2.9265 | 8.14 × 10⁻¹⁰ |
| SFN | 3.7239 | 1.54 × 10⁻⁹ |
| ACTG2 | 3.0293 | 8.70 × 10⁻¹² |
| HSPA6 | 3.5985 | 6.27 × 10⁻⁹ |
| TAGLN | 2.3661 | 4.54 × 10⁻⁸ |
| MAP1A | 2.1205 | 8.78 × 10⁻⁷ |
| GNGT2 | 2.5802 | 3.62 × 10⁻⁶ |

TABLE 2
List of top 10 downregulated genes
in ASR488-treated TCCSUP cells.

| Gene symbol | Log₂ (fold-change) | P-value |
|---|---|---|
| DDX60 | −2.5069 | 1.59 × 10⁻⁸ |
| GBP4 | −2.2704 | 2.98 × 10⁻⁸ |
| BBOX1 | −2.2384 | 7.26 × 10⁻⁶ |
| RSAD2 | −2.2235 | 9.71 × 10⁻⁸ |
| OASL | −1.9737 | 8.15 × 10⁻⁷ |
| FOS | −1.9465 | 9.91 × 10⁻⁵ |
| IFIT2 | −1.8473 | 1.66 × 10⁻⁶ |
| CMPK2 | −1.8470 | 8.78 × 10⁻⁷ |
| STEAP4 | −1.8141 | 2.70 × 10⁻⁵ |
| IFI44L | −1.9791 | 1.04 × 10⁻⁵ |

TABLE 3
Primers for top 10 upregulated genes in the quantitative PCR analysis.

| Gene | | | | |
|---|---|---|---|---|
| name | Forward primer, 5′-3′ | | Reverse primer, 5′-3′ | |
| CPEB1 | CCTTTCTTCTGTCG | SEQ ID | TTCTGGTTCCGCATCAG | SEQ ID |
| | AGATCAGG | NO: 1 | G | NO: 11 |
| CYP4F11 | CCCTGTTGACTTCT | SEQ ID | AAGGCCAGAGTAACCG | SEQ ID |
| | CTAATCTCTTC | NO: 2 | AGTG | NO: 12 |
| SPHK1 | CCTGCAGCCCACT | SEQ ID | GCTTAGCTGGGAGTCCA | SEQ ID |
| | GATAAAT | NO: 3 | CAT | NO: 13 |
| IL11 | GGGACCACAACCT | SEQ ID | GCAGCCTTGTCAGCACA | SEQ ID |
| | GGATTC | NO: 4 | C | NO: 14 |
| SFN | GACACAGAGTCCG | SEQ ID | ATGGCTCTGGGGACAC | SEQ ID |
| | GCATTG | NO: 5 | AC | NO: 15 |
| ACTG2 | GCGAGGGATCCTA | SEQ ID | TGTAGAAGGAGTGGTG | SEQ ID |
| | ACTCTCA | NO: 6 | CCAGA | NO: 16 |
| HSPA6 | CAAGGTGCGCGTA | SEQ ID | GCTCATTGATGATCCGC | SEQ ID |
| | TGCTAC | NO: 7 | AACAC | NO: 17 |
| TAGLN | GGCTTGGAGGGAA | SEQ ID | AGCCTGCCTGAAATGCA | SEQ ID |
| | GTTGG | NO: 8 | C | NO: 18 |
| MAP1A | CCTCCATGAGAGG | SEQ ID | CTCACTCTGGTGGCAAA | SEQ ID |
| | CTTCCT | NO: 9 | GG | NO: 19 |
| GNGT2 | TAGGGGCCGGTCT | SEQ ID | CCTTCCCACCTCTGAGC | SEQ ID |
| | AGGACT | NO: 10 | AT | NO: 20 |

### Example 2: ASR490, a Small Molecule, Overrides Aberrant Expression of Notch1 in Colorectal Cancer

Hyper-activation of Notch1 plays a significant role in the pathogenesis of cancer. Activation is triggered by binding of ligands to the receptor, which leads to protease (TACE or Kuzbanian proteases) driven sequential cleavages of the receptor followed by cleavage by γ-secretase. The cleaved Notch receptor intracellular domain (NICD) subsequently translocate to the nucleus, which induces the transcriptional activation of Notch target genes, such as HES1. Cleavage of NICD initiates a signaling cascade that has multiple interactive points with other oncogenic pathways. Moreover, HES1 activation has been shown to promote CRC cell resistance to 5-Fu by inducing EMT. Notch induction also activates several other oncogenic pathways and negatively affects pro-apoptotic pathways leading to activation of cell proliferation genes.

In colorectal cancer, Notch1 signaling is a major pathway that governs cancer cell differentiation and proliferation. Its dysregulation has been frequently associated with CRC pathogenesis, which is the second leading cause of cancer death in men and women. Although recent advances in CRC treatment have resulted in dramatic reductions in CRC-related death, CRC-related morbidity in young adults and chemoresistance to existing therapies is a major challenge in curing patients with CRC. The CRC incidence rate in adults aged ≥50 years decreased by 32%, while these incidence rates increased by 22% among adults aged <50 years.

Although, screening at an early stage can significantly improve survival, most of the CRC patients are diagnosed at an advanced stage. Neoadjuvant therapy before surgery, which is followed by chemotherapy, are recommended for such patients. However, pharmacological therapy often is associated with toxic and harmful side effects and patients eventually develop chemoresistance. Changes in cell signaling patterns, such as upregulation of expression or aberrant activation of several important genes such as anti-apoptotic factors (BCL-2 and BCL-XL), survival signaling, and EMT signaling, have been shown to be the main causative factors of chemoresistance in colorectal cancer.

Mutations in negative regulatory region (NRR) have been attributed to ligand-independent activation of Notch1 and resulted in aggressive malignancies. NRR is termed as an activation switch of Notch1 receptor. mAbs targeting NRR have shown promise by inhibiting Notch1 cleavage, which resulted in degradation of NICD. However, there is no report of compounds specifically binding to NRR and affecting Notch1 signaling. Increasing incidences of chemoresistance to existing therapies in advance colorectal cancer and importance of Notch1 signaling in maintenance of oncogenic phenotypes via uncontrolled proliferation, loss of apoptosis, and advancement to metastasis in colorectal cancer make it imperative to further broaden the current treatment paradigm by developing plant-derived novel small molecules, which have low toxicity profile, can target Notch1 signaling and its aberrant activation, and thus overcome these challenges.

Identified herein is a small molecule, ASR490, using structure-activity relationship studies focused on the Withaferin A analogs. ASR490 effectively inhibits colorectal cancer cell growth in both in vitro and in vivo models. The results also indicate that ASR490 effectively suppressed Notch1 signaling, which resulted in inhibition of EMT in colorectal cancer. Targeting the multifaceted functions of Notch1 receptor and several interlinked signaling pathways in colorectal cancer with a plant-derived potent small molecule presents a promising approach for the treatment of colon cancer.

Materials and Methods

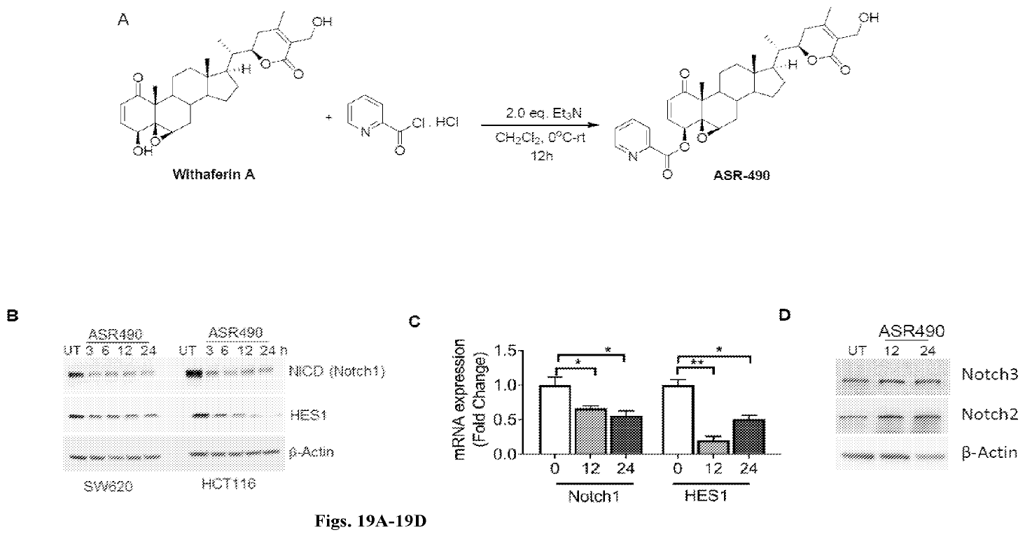

Synthesis of ASR490. ASR490 (Pyridine-2-carboxylic acid {17-[1-(5-hydroxymethyl-4-methyl-6-oxo-3,6-dihydro-2H-pyran-2-yl)-ethyl]-10,13-dimethyl-1-oxo, 4,6,7,8,9,10,11,12,13,14,15,16,17-tetradecahydro-20-oxacyclopropa[5,6]cyclopenta[a]phenanthren-4-yl}ester) was synthesized starting from Withaferin A (4β,5β,6β,22R)-4,27-Dihydroxy-5,6:22,26-diepoxyergosta-2,24-diene-1,26-dione) according to a synthetic strategy shown herein (material Section1 and FIG. 19A) with modifications in earlier reported protocols. Briefly, to a stirred solution of Withaferin A (0.470 g, 1.0 mmol) and triethylamine (0.278 mL, 2.0 mmol) in CH₂Cl₂ (10.0 mL) at 0° C. under nitrogen atmosphere was added pyridine-2-carbonyl chloride hydrochloride (0.195 g, 1.10 mmol) and the resulting reaction mixture was stirred overnight at room temperature. After completion of the reaction as indicated by TLC, the reaction mixture was quenched with saturated NaHCO₃ solution (5 mL). The organic layer was separated, followed with extraction of aqueous layer with CH₂Cl₂ (2×10 mL). The combined organic layers were washed with brine (20 mL), dried over anhydrous Na₂SO₄ and concentrated under reduced pressure to afford the crude ASR490 which was purified by silica gel column chromatography with eluent ethyl acetate:hexane (2:8) to afford the pure ASR490 (0.471 g, 82%) as a white solid The compound was characterized by NMR and MS and its purity (?98%) was determined by HPLC. ¹H NMR (600 MHz, CDCl₃): δ 8.78 (d, J=4.2 Hz, 1H), 8.15 (d, J=7.8 Hz, 1H), 7.86 (td, J=7.8, 1.2 Hz, 1H), 7.52-7.48 (m, 1H), 6.94 (dd, J=6.0, 4.2 Hz, 1H), 6.22 (d, J=10.2 Hz, 1H), 5.30-5.20 (m, 2H), 4.50-4.46 (m, 1H), 4.45-4.35 (m, 2H), 4.14 (q, J=7.2 Hz, 1H), 3.78 (d, J=6.0 Hz, 1H), 3.25 (1H, brs), 2.60-2.50 (m, 1H), 2.20-2.15 (m, 1H), 2.05 (2s, 6H), 2.00-1.98 (m, 1H), 1.90-1.85 (m, 1H), 1.70-1.65 (m, OH, 3H), 1.55-1.48 (m, 2H), 1.40 (s, 3H), 1.30-1.25 (m, 3H), 1.05-1.00 (m, 3H), 0.72 (s, 3H). ESI-MS m/z 576 (M+H)⁺.

Cell culture and supplies: HCT116, SW-620, TCCSUP, UMUC3, HT1376, 5637, T24 and RT4 cells were purchased from ATCC (American Type Culture Collection; Manassas, Va., USA). HCT116, T24, RT4 were maintained in McCoy's medium, TCCSUP, UMUC3, HT1376 in EMEM, SW620 in DMEM and 5637 in RPMI medium, respectively, and supplemented with 10% FBS and penicillin (100 units/mL) and streptomycin (100 units/mL; Millipore Sigma, St Louis, Mich., USA) in the presence of 5% CO₂ at 37° C. pCMV6-NOTCH1, vector pCMV6-Entry (NOTCH′ (NM_017617) Human ORF Clone; Origene) and NOTCH1 Human siRNA Oligo Duplex were obtained from Origene Technologies Inc. (Rockville, Md., USA). Lipofectamine 2000 reagent was used following the manufacturer's (Cat #11668019; ThermoFisher Scientific) instruction, transfection with overexpression vectors was performed with 500 ng plasmid concentration, while the siRNA was used in 25 nM concentration. Cells were allowed to be transfected for 48 hrs and later harvested or treated for further analysis. Neomycin (1 μg/mL) selection media was used to cultivate Notch1-overexpressing HCT116 clones (C1, C2, C3, C4, C5).

Cell proliferation and colony formation assay: The growth inhibitory effect of ASR490 (reconstituted in 10 mM DMSO) was determined by the MTT (3-[4, 5-Dimethylthiazol-2-yl]-2, 5-diphenyltetrazolium bromide) assay. Six biological replicates used for MTT assays and it was repeated twice for each experiment. Colon and bladder cancer cell lines were treated with varying concentrations of ASR490 (0-1.6 μM). The anchorage-independent growth assay was performed and repeated in triplicate.

Binding Studies. The protein-ligand binding was first studied by cellular thermal shift assay (CETSA) by following previously described protocol. Briefly, the cells (3×10⁶) were treated with ASR490, incubated at different temperatures (38-55° C.) to denature and precipitate proteins, performed cell lysis and centrifuged at 13000 g for 10 min to collect the soluble fraction. Equal amount of cell lysate was used for ELISA with NRR antibody (Cat: NBP2-62557; Novus Biologicals). GloMelt Thermal Shift Protein Stability assay was performed as per the kit instructions (GloMelt™ Thermal Shift Protein Stability Kit; Biotium; Fermont, Calif.). Briefly, a qPCR reaction was setup with the purified NRR protein (Origene technologies: TP606288), the GloMelt fluorescent dye and ASR490 (10 μg per reaction). A protein melt run profile was generated and Tm (melting temperature) was calculated using DNA melt curve software. To analyze protein melting, the Tm was considered at the lowest −dF/dT value (at the lowest point on the curve).

Molecular Docking studies. For molecular docking studies, the structure of NRR domain was downloaded with resolution 2 Å from the RCSB database (PDB ID: 3ETO). All bound crystal water molecules and ligands were removed prior to building missing residues through homology modeling using Modeller 9v15. Simultaneously, the structure of ASR490 we was built and optimized using Marvin sketch workspace (arXiv.org). The NRR structure was relaxed using Chiron and Gaia for subsequent docking studies with ASR490 compound. To evaluate the extent of interaction between ASR490 and the NRR domain of Notch1 receptor, molecular docking was performed using MedusaDock. H-bond interactions between Notch1-NRR domain and ASR490 compound as blue dotted lines. Notch1-NRR domain is shown as carton with α-helices in cyan, (3-sheets in magenta, and loops in deep salmon color. ASR490 is shown in green licorice representation and water molecules mediating the interaction between NRR and ASR490 are shown in red spheres.

Flow cytometry analysis: 0.3×10⁶ cells seeded in a 6 well plate and were cultured until 70-80% confluence was achieved. The cells were then treated with ASR490 for 24 h. To quantify apoptosis, flow cytometry analysis of the Annexin V-FITC against Propidium Iodide (PI) assay was performed following a previously described protocol. The Apoptosis detection kit was purchased from BD Pharminogen™, San Diego, Calif., USA. All experiments were repeated in triplicate to achieve statistical relevance.

Cell invasion and migration assays: The invasive capability of pCMV/HCT116 and Notch1/HCT116 was evaluated in Boyden chambers, as described in earlier studies. HCT 116, SW-620, pCMV/HCT116, and Notch1/HCT116 cells were analyzed for migration capability with protocols already described in an earlier study. All experiments were performed in triplicate to achieve statistical relevance.

Protein extraction and western blotting: Mammalian Protein Extraction Reagent (Thermo Scientific, Rockford, Ill., USA) was used to extract total protein from pCMV/HCT116, C₄, and C₅ cells as well as bladder cancer cells were prepared with the according to the manufacturer's instructions. Western blotting was performed using specific antibodies against Notch1 (Cat: 3447S), Bcl-2 (Cat: 2872S), E-cadherin (Cat: 3195S), N-cadherin (Cat: 13116S), Snail (Cat: 3879S), β-catenin (Cat: 8480S), NF-κB (p65), Bax (Cat: 5023S), cleaved PARP (Cat: 9541L) (Cell Signaling Technology; Danvers, Mass., USA), and β-actin (Santa Cruz Biotechnologies, Dallas, Tex., USA), HES1 (Genescript; Lot QC1851, Piscataway, N.J., USA). Actin presented in the images represent the loading control for one or more markers from same cell lysates. Chemiluminescence was used to detect the positive bands on the membrane.

Xenograft studies: Six- to eight-week-old BALB/c athymic nude mice (nu/nu) (Jackson Laboratory: Bar Harbor, Me., USA) were subcutaneously injected with pCMV/HCT116 and C₄ (1×10⁶ cells). The monitoring and measurements were performed.

Immunohistochemistry (IHC): The tumor samples from the pCMV/HCT116 and Notch1/HCT116 xenografts were subjected to IHC analysis. Primary antibodies against Ki67, Notch1, HES1, and p65 were used in this study.

Statistical analysis: The experimental data is presented as the mean±standard deviation (SD or SEM). Unpaired Student's t-test was used to determine the significance of the differences between different test groups. The significant differences were established at p<0.05. Prism 6 software purchased and licensed from GraphPad Software Inc, La Jolla, Calif., USA was used to perform the statistical analyses.

ASR490 Specifically Inhibits Notch1-Mediated Survival of CRC Cells.

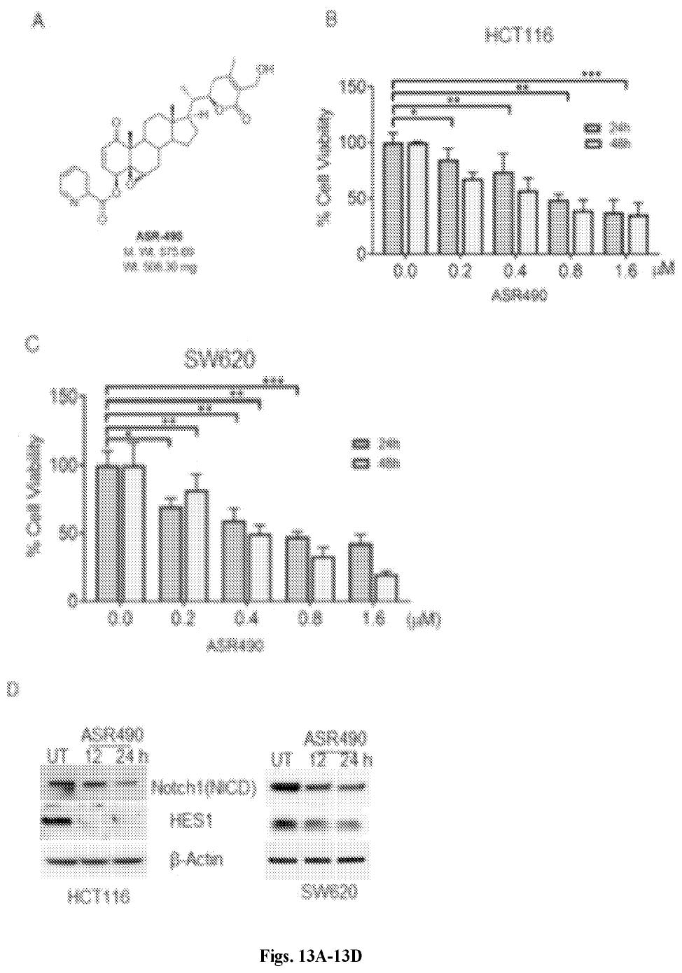

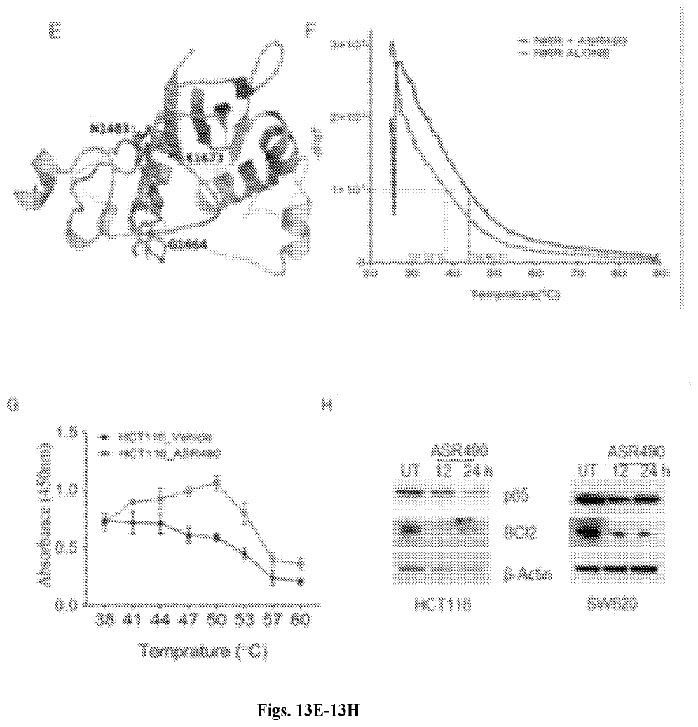

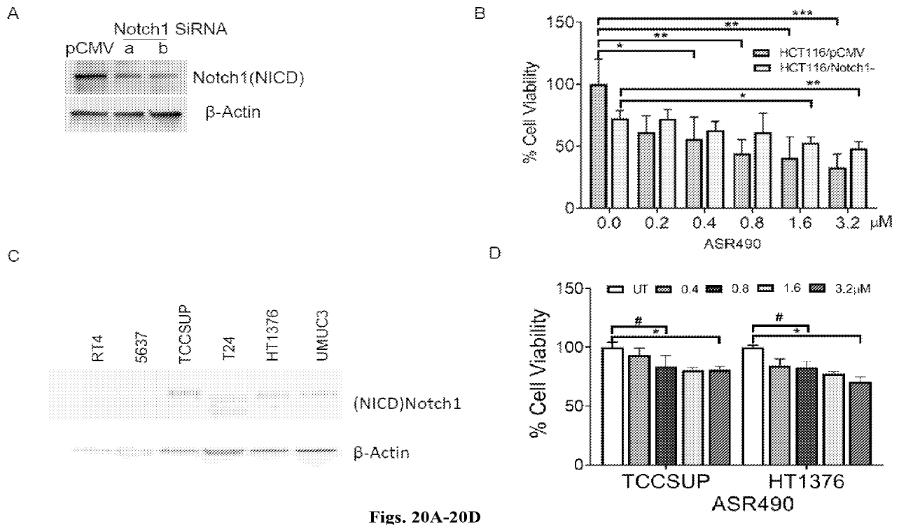

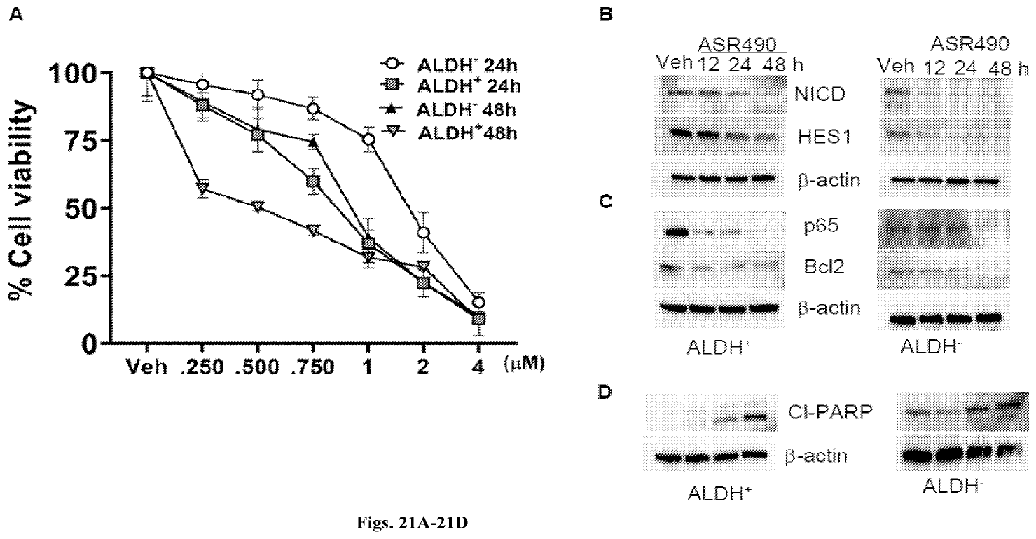

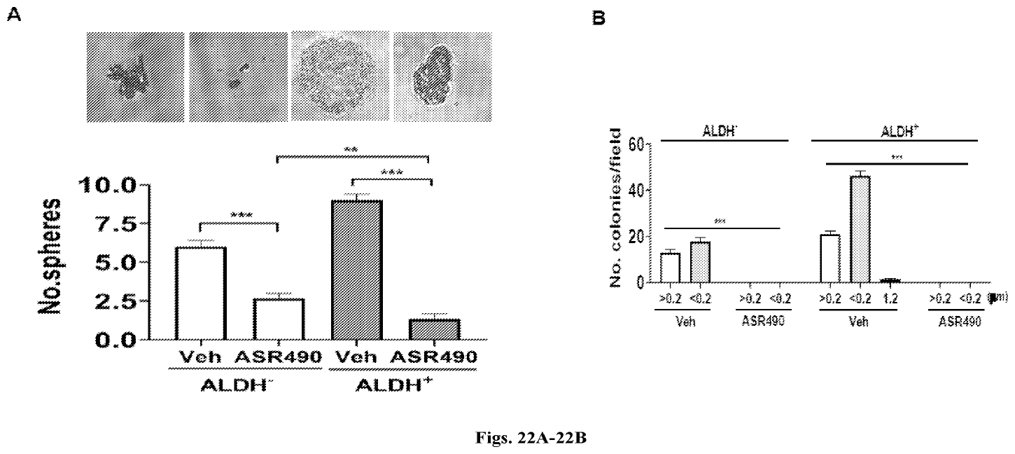

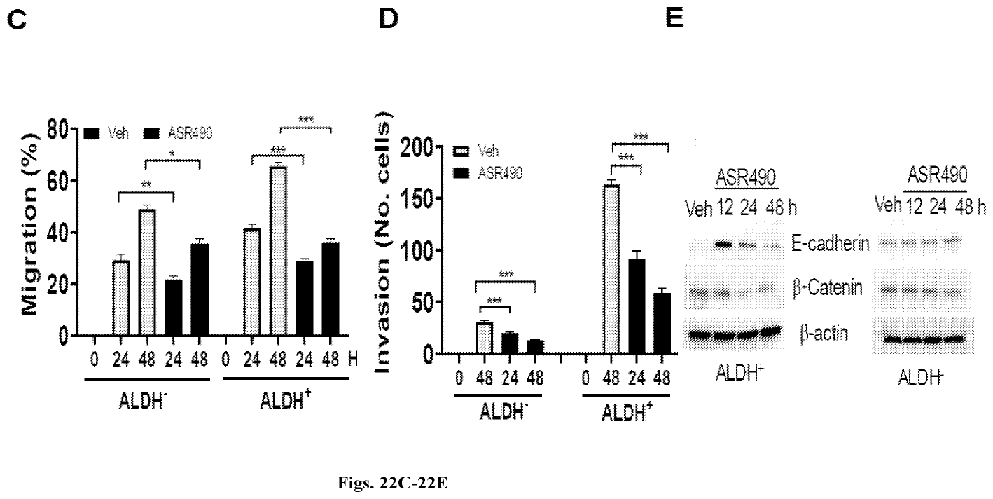

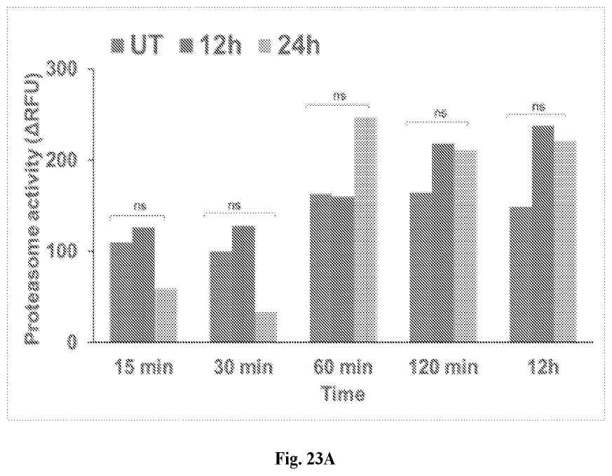

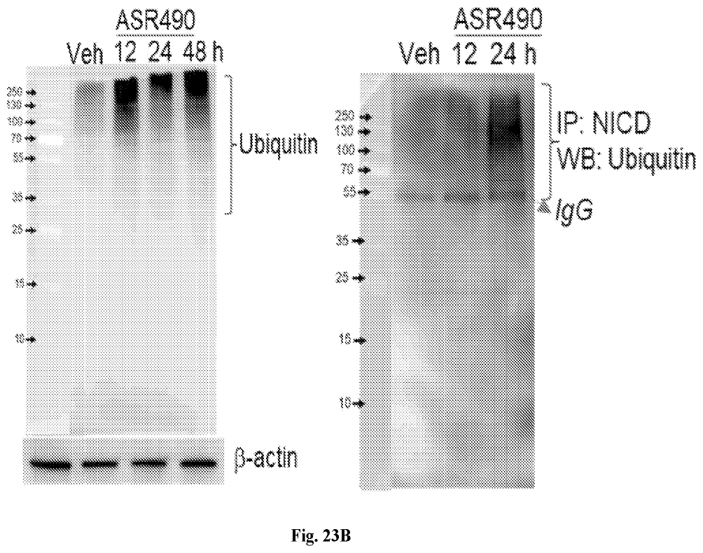

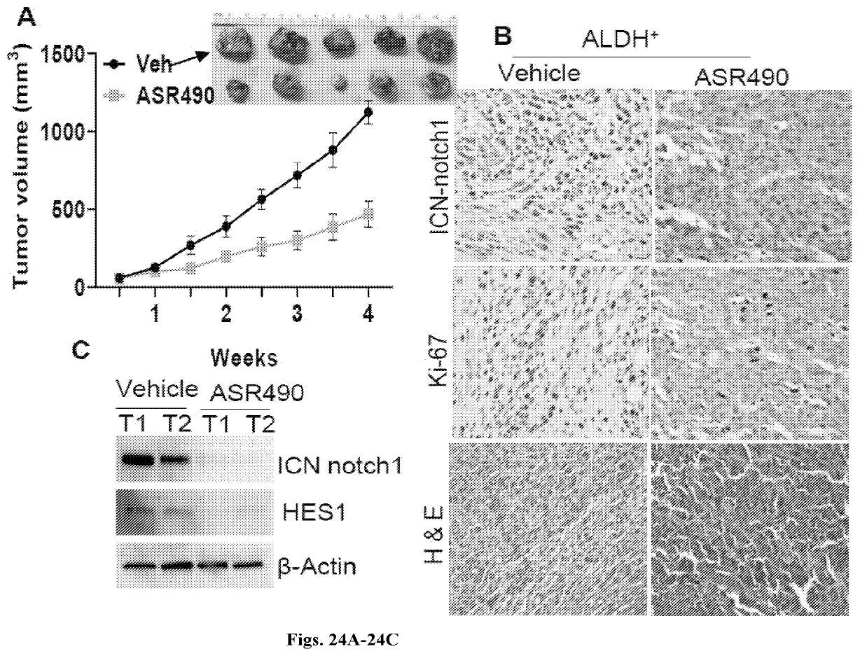

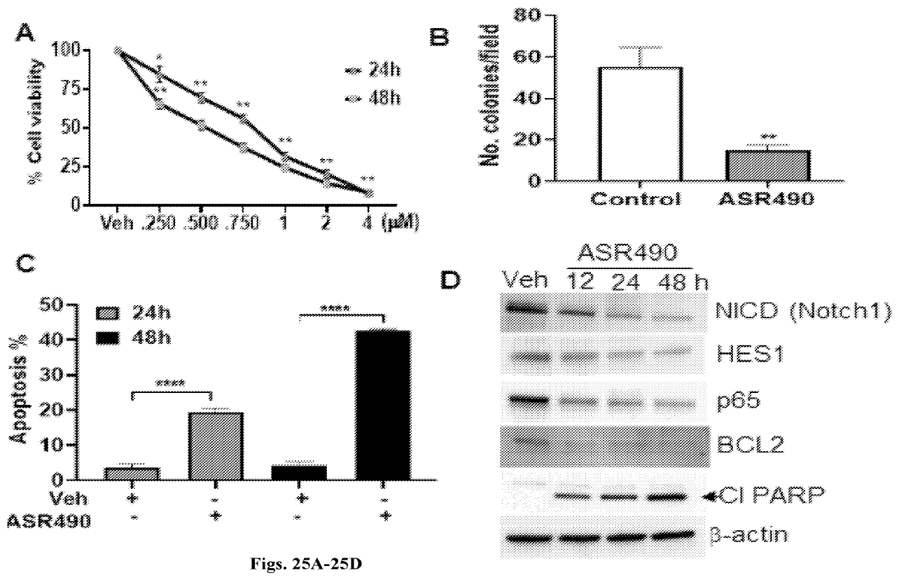

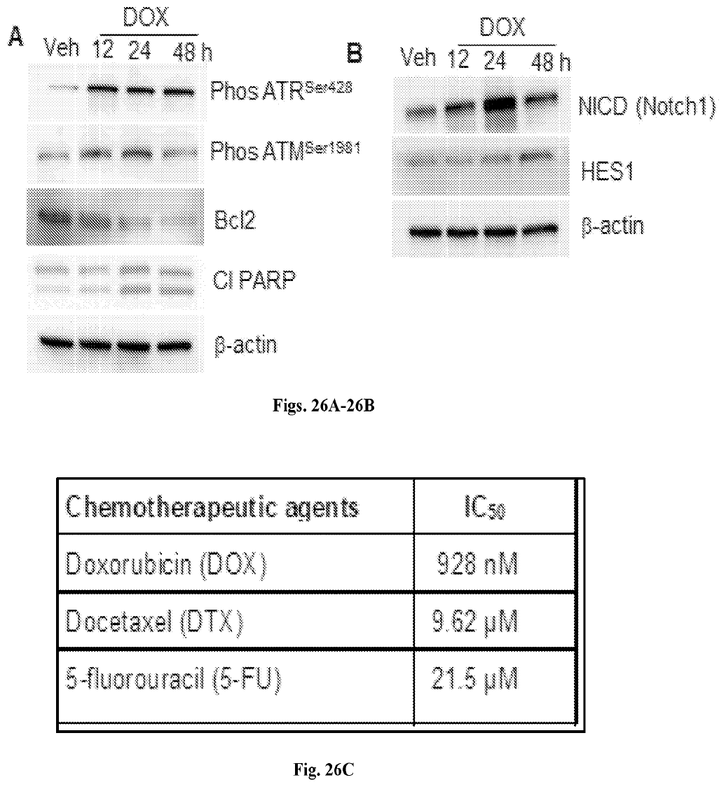

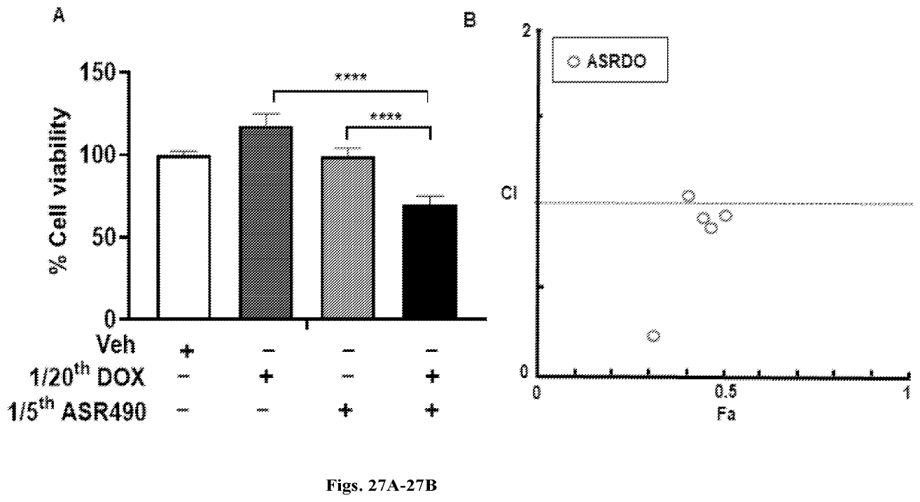

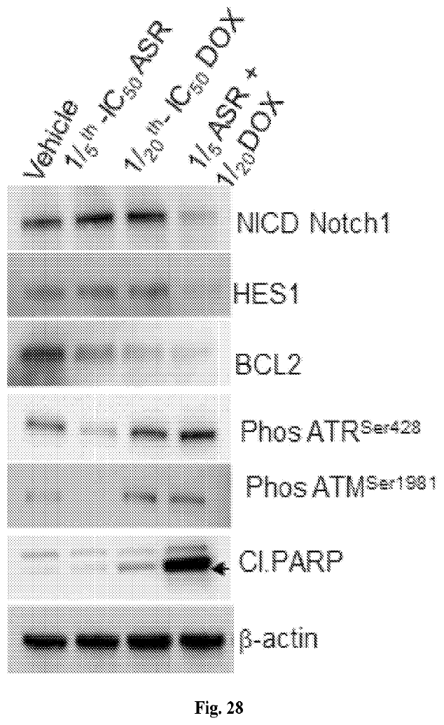

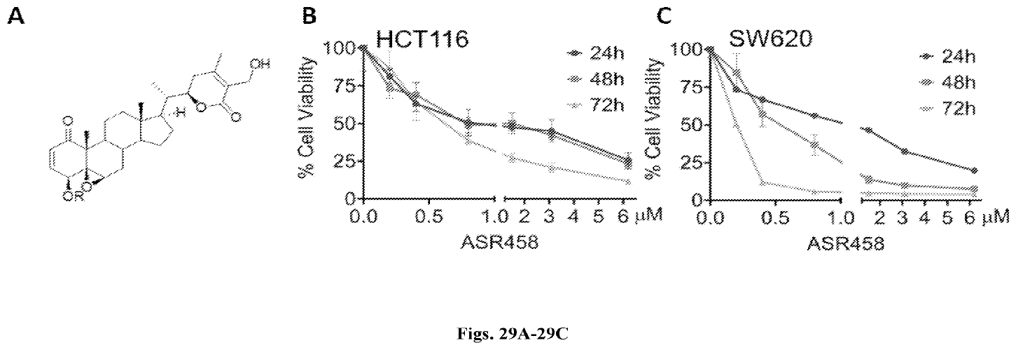

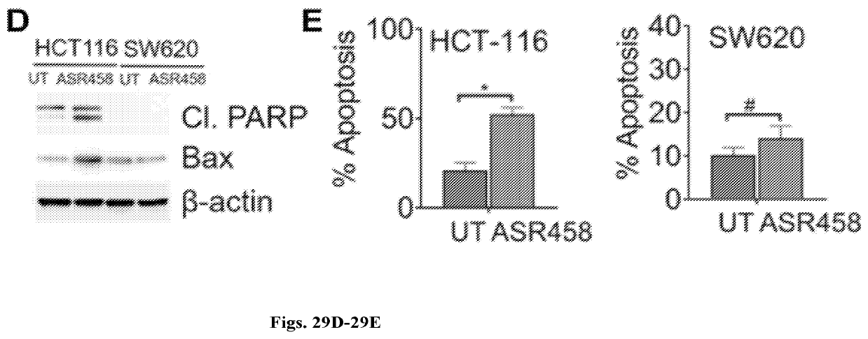

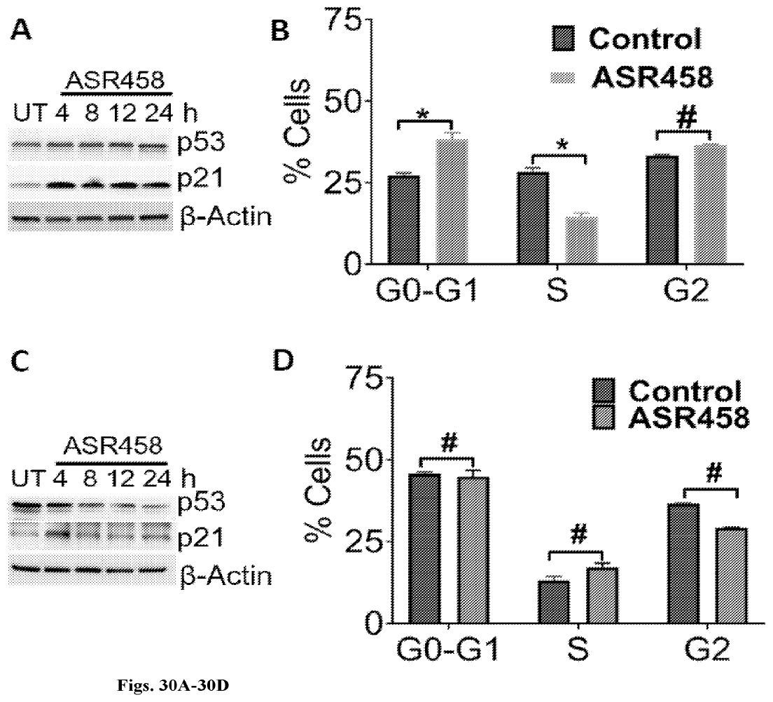

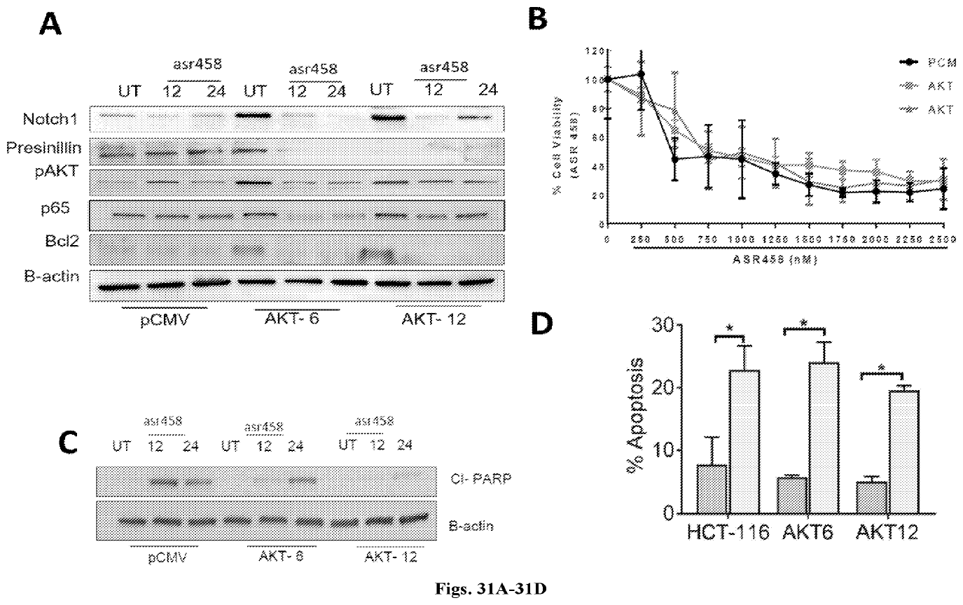

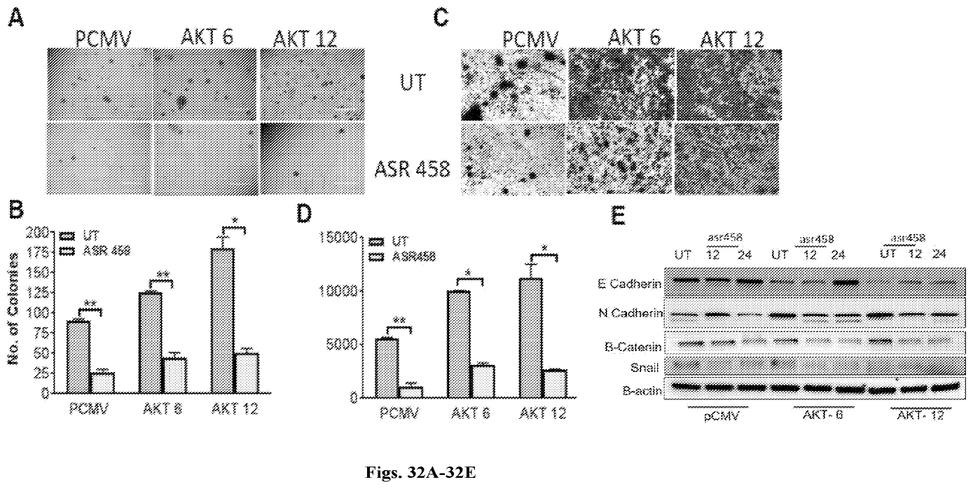

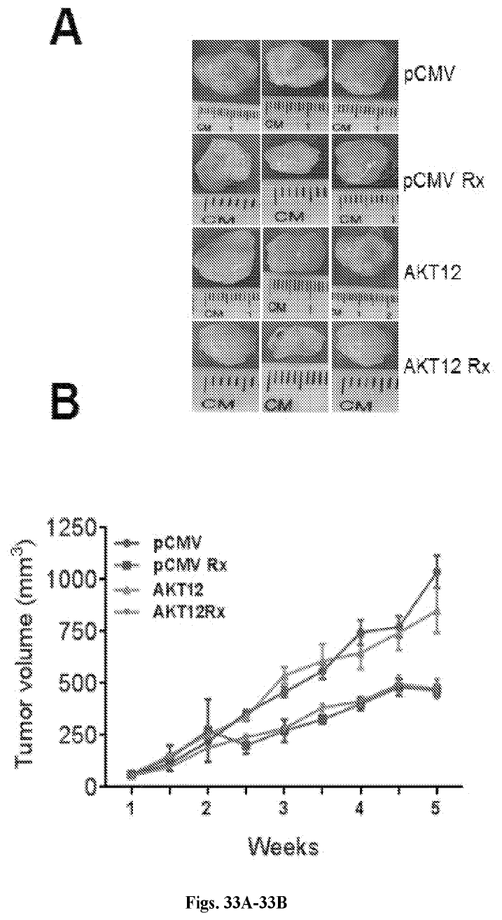

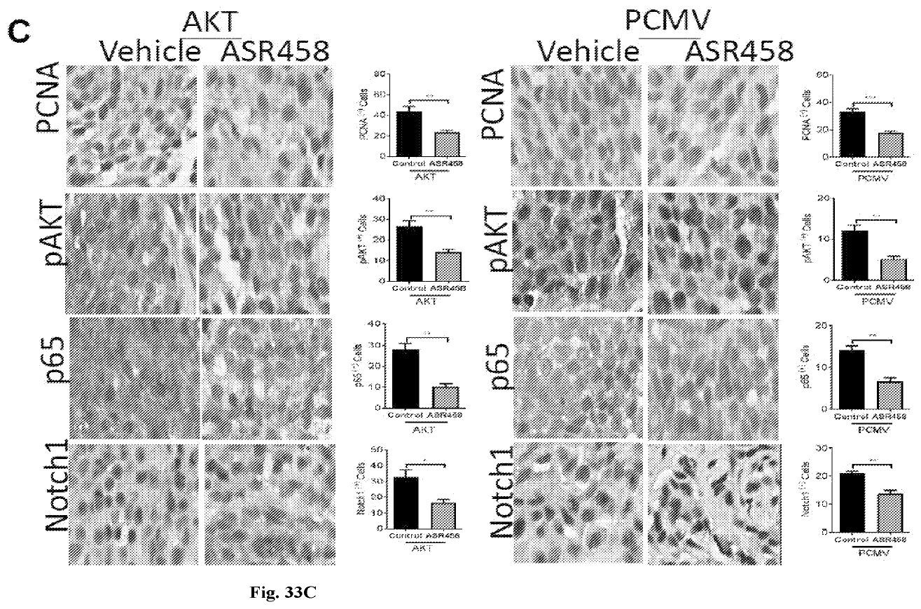

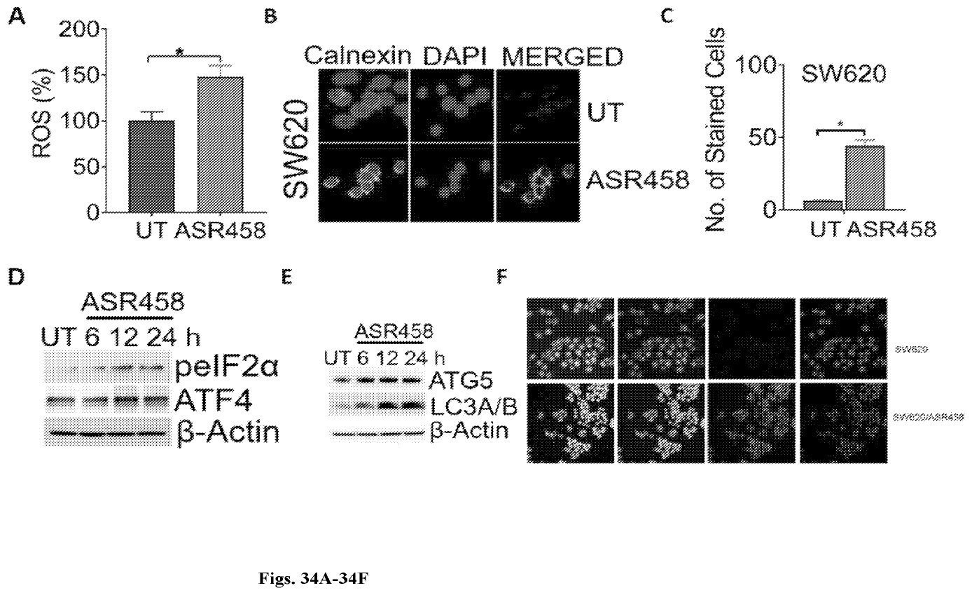

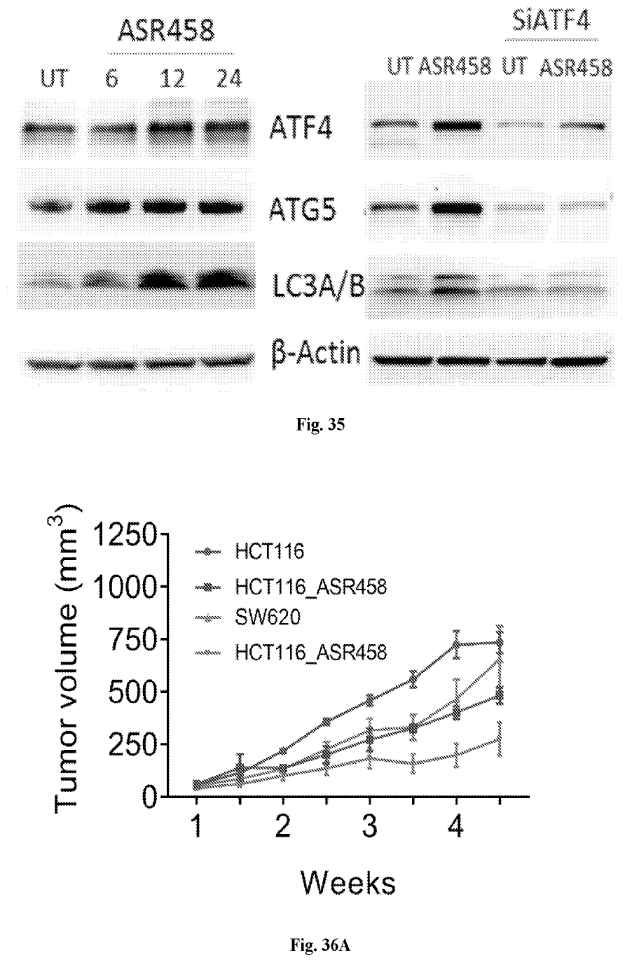

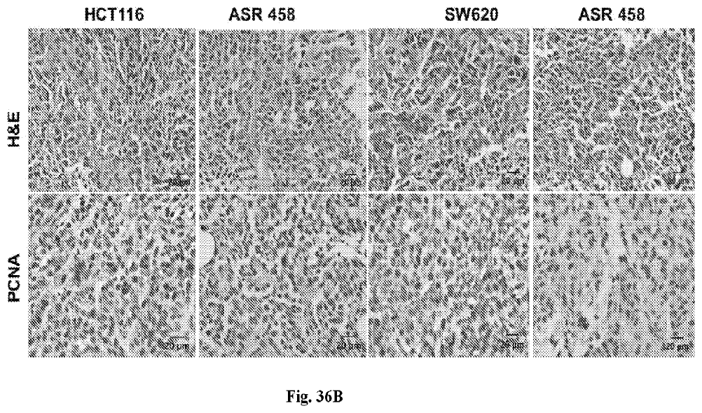

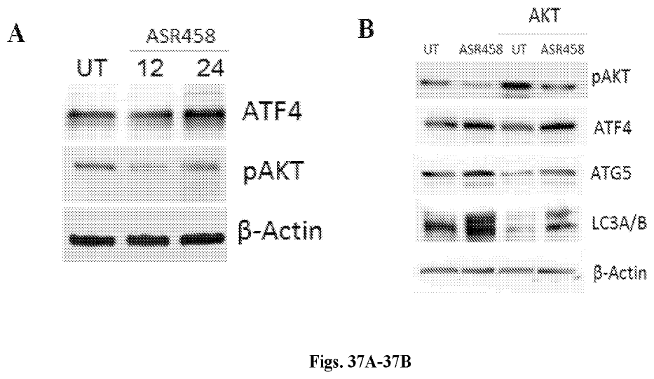

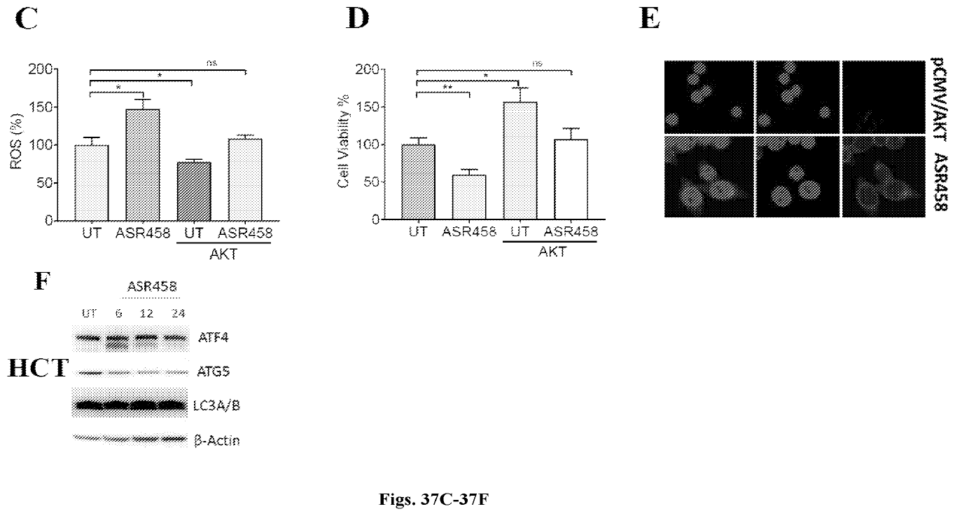

To examine the therapeutic potential of ASR490 (FIG. 13A). in CRC, we assessed the cell viability of ASR490-treated HCT116 and SW620 using the MTT assay. Cell viability was significantly reduced with 24-h (HCT 116, IC₅₀: 750 nM; p=0.007 and SW-620, IC₅₀:1.2 μM; p=0.0008) and 48-h (HCT 116, IC₅₀: 600 nM; p=0.005 and SW-620, IC₅₀: 850 nM; p=0.007) treatment (FIGS. 13B, 13C). To determine the molecular mechanism by which ASR490 inhibits the growth of CRC cells (HCT116 and SW620), the cell lines were treated with ASR490 and immunoblot analysis was performed. Significant downregulation in the expression of NICD and its downstream effector HES1 protein were observed in HCT116 and SW620 cells (FIG. 13D; FIG. 19B). An apparent decline in Notch1 and HES1 mRNA expression was observed (FIG. 19C), whereas no change in Notch2 and Notch3 expressions were seen in ASR490-treated HCT116 cells (FIG. 19D). To confirm that ASR490 inhibits the CRC cell growth through Notch1, Notch1 expression was silenced by siRNA in HCT116 cells (FIG. 20A), then the cells were treated with vehicle or ASR490. As seen in FIG. 20B, ASR490 failed to inhibit the growth of Notch1 silenced HCT116 cells as compared to scrambled transfected HCT116 cells. Bladder Cancer cells have low basal level of Notch1, these cells were treated with ASR490, which failed inhibit their growth (FIGS. 20C and 20D). These two experiments indicate that Notch1 can be a target for ASR490.

Next, to analyze whether ASR490 binds directly to Notch1, molecular docking studies were performed. The CASTp predictions confirmed binding sites of ASR490 in Negative Regulatory Region (NRR) of Notch1 (FIG. 13E). The catalytic pocket in NRR is lined by the residues: Lys-1462, Cys-1464, Asp-1479, Cys-1480, Leu-1482, Asn-1483, Ala-1708, Gly-1711, Leu-1713, Asn-1714, Ile-1715, Tyr-1717, Lys-1718, Ile-1719, and Glu-1720. The estimated binding energy between the NRR domain and ASR490 was −52.55 kcal/mol which signifies strong interaction between ASR490 and NRR domain. The residue-wise interaction analysis estimated three hydrogen-bond interactions between ASR490 and NRR residues Asn-1483, Glu-1673, and Gly-1664 mediated by water molecules (FIG. 13E). To further confirm the binding at protein level we performed Protein Thermal Shift differential scanning fluorimetry assay with purified NRR protein (FIG. 13F). The results indicate that an increased stabilization of NRR protein in presence of ASR490 than with vehicle (DMSO). Further, traditional CETSA was performed and NRR specific antibody was used in ELISA as detection method. The absorbance profile (495 nm for TMB substrate) of ASR490-treated HCT116 cells confirmed that ASR490 binds directly to NRR (FIG. 13G). To further confirm that the inhibition of Notch1 activation alters the expression of key genes that regulate cancer cell survival signaling in CRC cells, the effect of ASR490 treatment on pro-survival genes was analyzed. As shown in FIG. 13H, ASR490 treatment significantly inhibited p65 and Bcl-2 expression in colorectal cancer cells.

Notch1 Inhibition Resulted in EMT Downregulation in CRC Cells

To examine whether Inhibition of Notch1 signaling facilitates induction of pro-apoptotic signaling, we performed apoptotic assays in ASR490 treated CRC cells. Induction of apoptosis in ASR490-treated HCT116 (19.9%, p=0.01; 24 h) as well as SW-620 (9.57%, p=0.011; 24 h) cells in FACS analysis showed significant apoptotic cell death (FIGS. 14A, 14B). A time-dependent up-regulation of the pro-apoptotic markers Bax and cleaved PARP expression was observed in ASR490-treated HCT116 and SW620 cells (FIGS. 14C, 14D). More importantly, inhibition of the migratory (25.18%, p=0.05; HCT116 and 32.36%, p=0.032; SW620) capability of CRC cells was observed in response to ASR490 treatment for 24 h (FIGS. 14E, 14F). Additionally, the time-dependent increase in the E-cadherin (an epithelial marker) and a significant decrease in mesenchymal markers N-cadherin and β-catenin expression were observed in CRC cells treated with ASR490 for both 12 and 24 h (FIGS. 14G, 14H).

ASR490 Overcame Notch1 Overexpression and Inhibited the Growth of Notch1/HCT116 Transfectants.

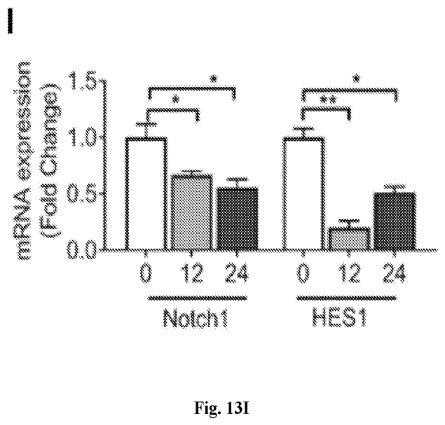

To assess the proliferative attribute of Notch1 in CRC, Notch1 expressing stable HCT116 cell lines, i.e. clones C1, C2, C3, C4, and C5, were generated (FIG. 15A). C4 and C5 were used for further studies as they expressed higher Notch1 compared to other clones. pCMV/HCT116 and Notch1/HCT116 clones C4 and C5 were assessed for cellular growth. Notch1 transfectants showed a significantly higher growth compared to the control (pCMV/HCT116) cells (C4: 42%, p=0.0226; and C5: 25.8%, p=0.0236; FIG. 15B). ASR490 treatment significantly inhibited cell growth in both C4 (IC₅₀: 800 nM; p=0.0016) and C5 (IC₅₀: 1.1 μM; p=0.0028) transfectants, showing the ability of ASR490 to override Notch1 mediated overgrowth of CRC cells (FIG. 15B). To understand the effect of Notch1 in increasing the tumorigenic capability of CRC cells, a colony forming assay was performed. The colony forming ability in Notch1 transfectants increased significantly (C4 −42%; p=0.0238 and C5 −32.4%; p=0.0238) compared to vector-transfected HCT116 cells. However, ASR490 treatment significantly reduced the colony forming ability of pCMV/HCT116 (22.3%; p=0.0169), C4 (38%; p=0.0406), and C5 (26.66%; p=0.0127) cells (FIG. 15C). Immunoblot and densitometry analysis of ASR490-treated transformants (C4 and C5) demonstrated inhibition of Notch1 and HES1 expression (FIGS. 15D, 15E). Additionally, p65 and BCl2 expression (survival markers) was downregulated (FIGS. 16A, 16B). Next, increase in expression of pro-apoptotic genes were observed, such as cleaved-PARP and Bax (FIG. 16C) along with induction of apoptosis in ASR490-treated pCMV/HCT116 (25.8%, p=0.0165) as well as C4 (13.4%, p=0.0102) and C5 (13.2%, p=0.0112) cells during FACS analysis (FIG. 16D). ASR490 overcame Notch1-induced EMT and decreased tumorigenicity of CRC cells.

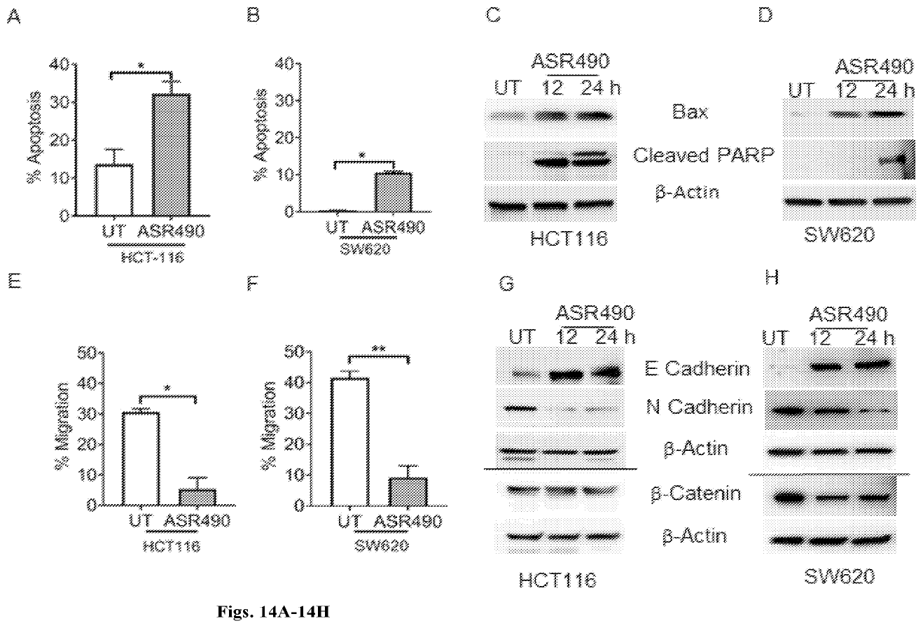

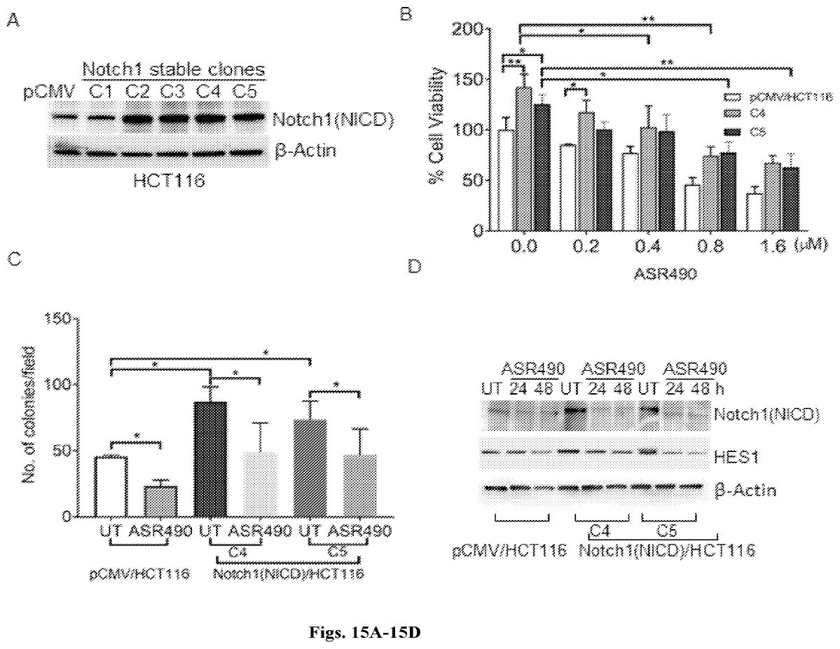

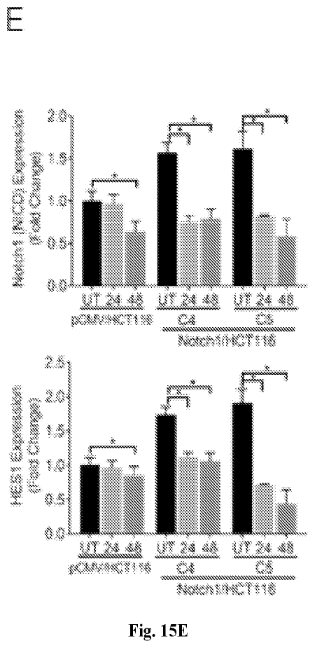

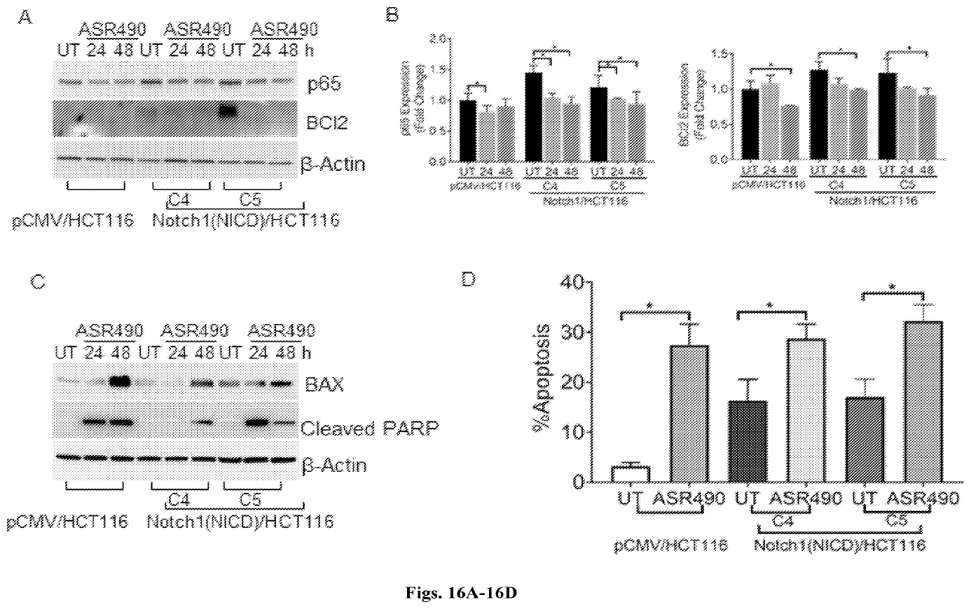

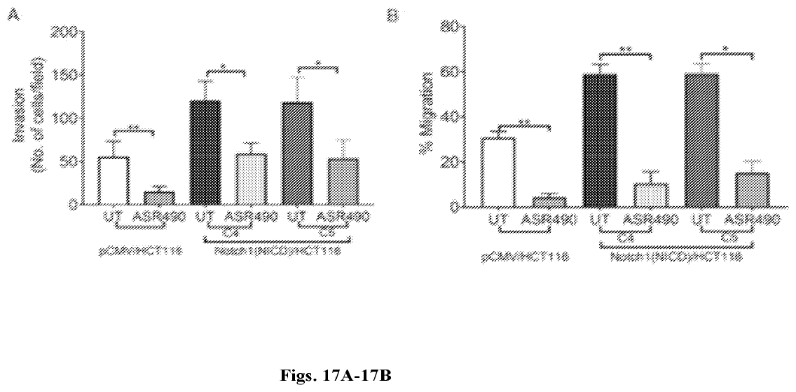

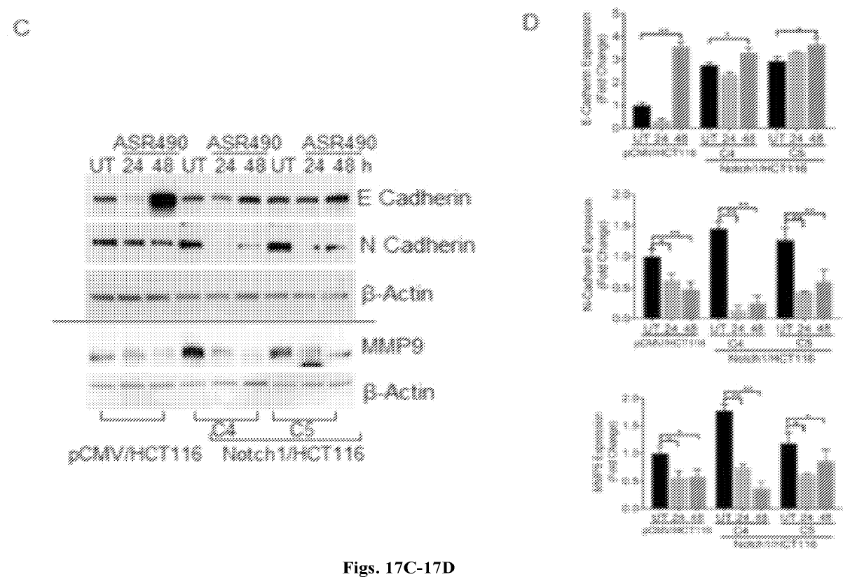

Next, whether Notch1 overexpression influences EMT signaling in Notch1/HCT116 cells was determined. The invasive capability in C4 and C5 cells increased by 65.5% (p=0.0316) and 63.5% (p=0.0253), respectively (FIG. 17A). Similarly, a 29% (p=0.036) and 28.68% (p=0.0474) increase was observed in the migratory capability of C4 and C5 CRC cells, respectively, (FIG. 17B) compared to pCMV/HCT116 cells. To analyze whether ASR490 treatment can inhibit the enhanced migratory and invasive capability of C₄ and C₅ cells, both transfectants were treated with the respective IC₅₀ doses of ASR490 for 24 h. A significant decline in the migratory potential of pCMV/HCT116 (25.11%; p=0.0073) and Notch1 transfectants (C4 −49.3%; p=0.0031 and C5 −44%; p=0.0130) and the invasive capacity of pCMV/HCT116 (40%; p=0.0047) and both C4 (60.75%; p=0.0305) and C5 cells (65.5%; p=0.0301) was observed (FIGS. 17A, 17B). Next ASR490-treated pCMV/HCT116, C4, and C5 cells were analyzed for expression of genes that regulate EMT. EMT markers such as N-cadherin, and MMP-9 were significantly downregulated, while the epithelial marker E-cadherin expression upregulated, which are hallmarks of EMT (FIGS. 17C, 17D). Notch1 plays an active role in the EMT process, and the results collectively indicate that ASR490 can overcome Notch1-induced EMT signaling in colorectal cancer cells.

ASR490 Overcomes Notch1 Induced Tumor Growth in Xenotransplanted Mice

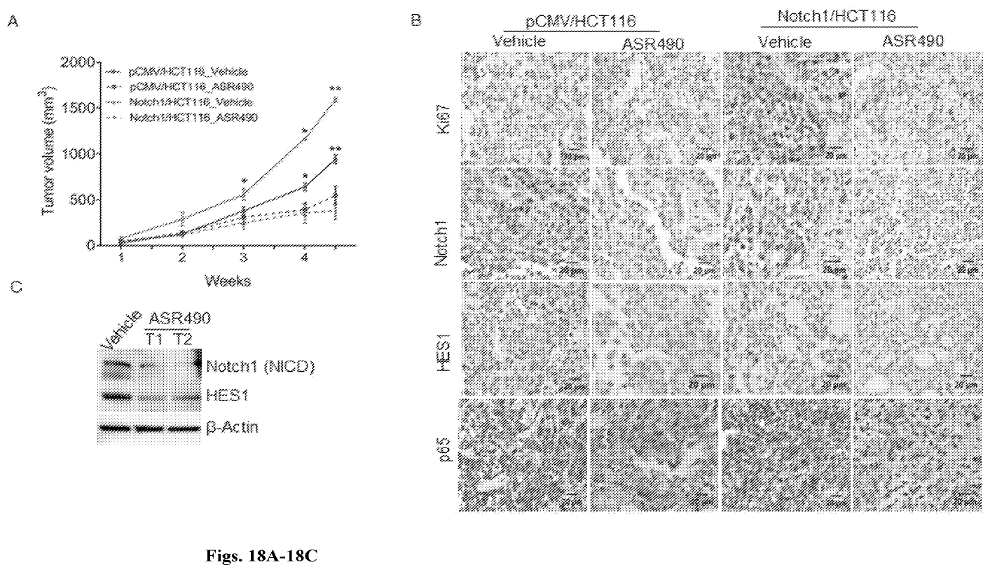

Earlier report shows that colorectal cancer xenografts with overexpression of AKT (Notch1 and AKT signaling are interlinked) are significantly aggressive compared with control-transfected colorectal cancer xenografts. To determine antitumor effect of ASR490, pCMV/HCT116 and Notch1/HCT116 (clone 4; C₄) cells were subcutaneously injected into nu/nu mice. The MTD was checked for ASr490 in nu/nu mice, and ASR490 was found to be safe till 500 mg/kg dose. Notch1/HCT116 tumors showed rapid and aggressive growth compared with pCMV/HCT116 tumors (FIG. 18A). On the other hand, significant tumor growth inhibition was noted in both the ASR490-treated (5 mg/kg of mouse body weight for 4 weeks) pCMV/HCT116 and Notch1/HCT116 (C4) xenografts.

The survival (p65, Notch1, HES1) and proliferation (Ki67) was assessed in both pCMV/HCT116 and Notch1/HCT116 (C4) tumors. Notch1 and HES1 expression was higher in Notch1/HCT116 tumors compared with pCMV/HCT116 tumors (FIG. 18B). ASR490 treatment resulted in significant reduction of Ki67 expression (cellular proliferation) in pCMV/HCT116, as well as Notch1-overexpressing Notch1/HCT116 (C4) tumors (FIG. 18B). In addition, ASR490 treatment significantly reduced the expression of the prosurvival marker and p65 in all tumors (FIG. 18B). Next, to assess the effect of ASR490 treatment on Notch1 signaling in xenografts, Notch1 and HES1 protein expression was analyzed in pCMV/HCT116 and Notch1/HCT116 (C4) tissues. Consistent with the IHC results, an inhibition in Notch1 and HES1 protein levels were observed (FIG. 18C).

The current study demonstrated that aberrant Notch1 overexpression causes CRC cells to grow rapidly and demonstrate aggressive migratory behavior and our newly identified small molecule, ASR490, overrides aberrant overexpression of Notch1 in in vitro and in vivo CRC models to achieve antiproliferative and antitumorigenic effects.

Modern treatment concepts in CRC are multimodal and use interdisciplinary approaches, including the use of adjuvant, neo-adjuvant chemotherapy, radiotherapy, and immunotherapy, are followed based on the CRC stage and localization. However, it is well understood that the need for optimization of adjuvant therapies and increasing instances of resistance in neo-adjuvant therapies warrant identification of new therapeutic targets and support the search for new compounds with low toxicity profiles and better bioavailability.

The current study has demonstrated that aberrant Notch1 overexpression causes colorectal cancer cells to grow rapidly and demonstrates aggressive migratory behavior, and our newly identified small molecule, ASR490, overrides aberrant overexpression of Notch1 in in vitro and in vivo colorectal cancer models to achieve antiproliferative and antitumorigenic effects.

Modern treatment concepts in colorectal cancer are multimodal and use interdisciplinary approaches, including the use of adjuvant, neoadjuvant chemotherapy, radiotherapy, and immunotherapy, which are followed based on the colorectal cancer stage and localization. However, it is well understood that the need for optimization of adjuvant therapies and increasing instances of resistance in neoadjuvant therapies warrant identification of new therapeutic targets and support the search for new compounds with low toxicity profiles and better bioavailability.

The recent progress in determination of the crystal structure of the NRR has improved the understanding of mechanisms that are responsible the self-inhibitory effects of HD domain on the processing and activation of NOTCH receptors. Recently, mAbs have been used to target NRR region in order to stabilize the region and prevent ligand-independent activation and wild-type Notch1 activation and thus decrease in NICD expression. In our results, molecular docking studies indicate ASR490 binds to NRR region of Notch1. Further, the CETSA and GloMelt protein thermal shift assay were performed with NRR-specific antibody of Notch1 on the purified NRR protein, and those results further confirmed that ASR binds to NRR of Notch1 and downregulated Notch1 expression. Similarly, Notch3 antibodies against NRR domain have shown to inhibit expressions of NICD and HES1, whereas the anti-LBD antibody failed to achieve that. It is possible that ASR490 can elicit the similar response as anti-NRR antibodies, although the exact mechanism needs to be elucidated in detail. In addition to downregulation of NICD expression, we have also seen inhibition of Notch1 gene expression. However, the exact mechanism by which ASR490 inhibits Notch1 gene expression is yet to be elucidated.

Notch1 activation is associated with early development of cancer, and activation of its downstream events such as overexpression of HES1 has been linked with colorectal cancer progression and metastasis. Silencing Notch1 activity through lentiviral-encoding Notch-1-siRNA and Notch1 inhibitors such as DAPT (N—[N-(3,5-difluorophenacetyl)-L-alanyl]-S-phenylglycine t-butyl ester) has demonstrated capability to induce apoptosis in colorectal cancer cells, proving that Notch1 can be an effective target for colorectal cancer management. However, the current landscape of inhibitors, particularly gamma secretase inhibitors (GSI) such as LY-411,575 or DAPT, can have unintended biological implications because of broad substrate profile of gamma secretase. Natural compounds such as Butein and more recently compounds isolated from Nerium indicum have been reported as inhibitors of Notch1. Keeping in mind the low toxicity profiles of compounds derived from natural sources and high bioavailability, results from our study showing the detrimental effect on Notch1 signaling by ASR490 derived from a natural compound are encouraging.

Notch1-mediated survival has been shown to be a primary driver of cell proliferation and tumor recurrence in vivo. Moreover, its aberrant activation has been found to be responsible for uncontrolled cellular growth in several cancer types. Inhibition of its expression and downstream signaling has resulted in induction of apoptosis and thus growth arrest in HT29 cells. A tripeptide of GSIs category inhibited the proliferation of MDA-MB231 cells, whereas natural compounds such as genistein induce apoptosis in cancer cells by downregulating survival signaling, particularly NF-κB expression. Similar alteration in survival as well as apoptotic signaling was seen in ASR490-treated colorectal cancer cells in the current study. The Notch1-overexpressing transfectants mimicking aberrant overexpression conditions also showed downregulation of proapoptotic and prosurvival markers, indicating that uncontrolled growth of colorectal cancer cells in the case of Notch1 activation can be managed by ASR490 treatment.

Notch1 signaling is also recognized as a major regulator of EMT in several cancer types including colon cancer. Activation of Notch1 signaling accelerates EMT by positively regulating Snail, a slug family protein, and repressing E-cadherin function. This in turn affects the progression of tumors in cancer cells. In addition, elevated HES1 expression has been correlated with several neoplastic conditions. Its interaction with multiple signaling pathways has been attributed to its contribution toward promotion of cell metastasis by evading tumor cell differentiation. Alleviation of Notch1-induced EMT can well be a direct result of the inhibition of Notch1/HES1/NFκB-p65 signaling. The ability of ASR490 to overcome Notch1 signaling and inhibit tumorigenic capacity was shown in our preclinical models of colorectal cancer. Our studies indicate that ASR490 is safe up to a dose of 500 mg/kg, which is 100 times more than the dose used in our efficacy studies indicating a high therapeutic index.

In summary, the results indicate that ASR490, a potent small molecule, overcomes Notch1-mediated prosurvival signaling and EMT, which resulted in growth inhibition in preclinical models of colorectal cancer. Additional studies can require optimizing the therapeutic efficiency of ASR490 that can lead its translation to clinical settings.

### Example 3: Endoplasmic Reticulum (ER), a Potential Therapeutic Target for Mutant p53 Colorectal Cancer

Inactivating p53 mutations contribute to tumor progression and treatment-resistance, resulting in poor patient survival in colorectal cancer patients (CRC). The goal of this study is to identify novel small molecules that therapeutically target mutant p53 in CRC.

The effects of small molecule (ASR458) on p53-wild type (p53-wt; HCT116) and mutant p53 (p53-mut; SW620) colon cancer cells were analyzed by phenotypic, molecular and in vivo assays.

ASR458 treatment significantly inhibited the proliferation of both HCT116 and SW620 cells at nM concentrations. In p53-wt HCT116 cells, ASR458 caused induction of p53 that resulted in caspase-mediated cell death in both in vitro and in vivo models. On the contrary, ASR458 treatment induced ER-stress signaling (i.e., phosphorylation of ERK and eIF2-α) in p53-mut SW620 cells, which triggered ATF4 activation and subsequent induction of cascade of autophagy events (Atg family proteins, LC3B and Lamp1), causing autophagy-mediated cell death. Silencing ER stress marker ATF-4, a key regulator of autophagy, caused resistance to ASR458 and abrogated autophagy signaling in SW620 cells. This indicated that induction of ER-stress is critical for the cytotoxic effects of ASR458 in p53-mut CRC. Preliminary knockdown studies indicate that silencing of ER markers causes resistance to ASR458 in vitro, further confirming ER-stress as the mechanism of ASR458 action in p53-mut CRC. ASR458 significantly inhibited the growth of SW620 tumors in xenograft. Tissue analysis confirmed the ER-stress signaling observed in vitro.

The results demonstrate ASR458 as a therapeutic agent with distinct targets in p53-wt and p53-mut CRC. This study also indicates that ATF4 mediated autophagy in unmanaged ER stress can reduce CRC pathogenesis. Further investigation into the pharmacokinetics and pharmacodynamics of ASR458 helps clinical translation of this agent.

SEQUENCES
SEQ ID NO: 1
CCTTTCTTCTGTCGAGATCAGG
SEQ ID NO: 2
CCCTGTTGACTTCTCTAATCTCTTC
SEQ ID NO: 3
CCTGCAGCCCACTGATAAAT
SEQ ID NO: 4
GGGACCACAACCTGGATTC
SEQ ID NO: 5
GACACAGAGTCCGGCATTG
SEQ ID NO: 6
GCGAGGGATCCTAACTCTCA
SEQ ID NO: 7
CAAGGTGCGCGTATGCTAC
SEQ ID NO: 8
GGCTTGGAGGGAAGTTGG
SEQ ID NO: 9
CCTCCATGAGAGGCTTCCT
SEQ ID NO: 10
TAGGGGCCGGTCTAGGACT
SEQ ID NO: 11
TTCTGGTTCCGCATCAGG
SEQ ID NO: 12
AAGGCCAGAGTAACCGAGTG
SEQ ID NO: 13
GCTTAGCTGGGAGTCCACAT
SEQ ID NO: 14
GCAGCCTTGTCAGCACAC
SEQ ID NO: 15
ATGGCTCTGGGGACACAC
SEQ ID NO: 16
TGTAGAAGGAGTGGTGCCAGA
SEQ ID NO: 17
GCTCATTGATGATCCGCAACAC
SEQ ID NO: 18
AGCCTGCCTGAAATGCAC
SEQ ID NO: 19
CTCACTCTGGTGGCAAAGG
SEQ ID NO: 20
CCTTCCCACCTCTGAGCAT
(polypeptide sequence of Notch1)
SEQ ID NO: 21
MPPLLAPLLCLALLPALAARGPRCSQPGETCLNGGKCEAANGTEACVCGG
AFVGPRCQDPNPCLSTPCKNAGTCHVVDRRGVADYACSCALGFSGPLCLT
PLDNACLTNPCRNGGTCDLLTLTEYKCRCPPGWSGKSCQQADPCASNPCA
NGGQCLPFEASYICHCPPSFHGPTCRQDVNECGQKPGLCRHGGTCHNEVG
SYRCVCRATHTGPNCERPYVPCSPSPCQNGGTCRPTGDVTHECACLPGFT
GQNCEENIDDCPGNNCKNGGACVDGVNTYNCRCPPEWTGQYCTEDVDECQ
LMPNACQNGGTCHNTHGGYNCVCVNGWTGEDCSENIDDCASAACFHGATC
HDRVASFYCECPHGRTGLLCHLNDACISNPCNEGSNCDTNPVNGKAICTC
PSGYTGPACSQDVDECSLGANPCEHAGKCINTLGSFECQCLQGYTGPRCE
IDVNECVSNPCQNDATCLDQIGEFQCICMPGYEGVHCEVNTDECASSPCL
HNGRCLDKINEFQCECPTGFTGHLCQYDVDECASTPCKNGAKCLDGPNTY
TCVCTEGYTGTHCEVDIDECDPDPCHYGSCKDGVATFTCLCRPGYTGHHC
ETNINECSSQPCRHGGTCQDRDNAYLCFCLKGTTGPNCEINLDDCASSPC
DSGTCLDKIDGYECACEPGYTGSMCNINIDECAGNPCHNGGTCEDGINGF
TCRCPEGYHDPTCLSEVNECNSNPCVHGACRDSLNGYKCDCDPGWSGTNC
DINNNECESNPCVNGGTCKDMTSGYVCTCREGFSGPNCQTNINECASNPC
LNQGTCIDDVAGYKCNCLLPYTGATCEVVLAPCAPSPCRNGGECRQSEDY
ESFSCVCPTGWQGQTCEVDINECVLSPCRHGASCQNTHGGYRCHCQAGYS
GRNCETDIDDCRPNPCHNGGSCTDGINTAFCDCLPGFRGTFCEEDINECA
SDPCRNGANCTDCVDSYTCTCPAGFSGIHCENNTPDCTESSCFNGGTCVD
GINSFTCLCPPGFTGSYCQHDVNECDSQPCLHGGTCQDGCGSYRCTCPQG
YTGPNCQNLVHWCDSSPCKNGGKCWQTHTQYRCECPSGWTGLYCDVPSVS
CEVAAQRQGVDVARLCQHGGLCVDAGNTHHCRCQAGYTGSYCEDLVDECS
PSPCQNGATCTDYLGGYSCKCVAGYHGVNCSEEIDECLSHPCQNGGTCLD
LPNTYKCSCPRGTQGVHCEINVDDCNPPVDPVSRSPKCFNNGTCVDQVGG
YSCTCPPGFVGERCEGDVNECLSNPCDARGTQNCVQRVNDFHCECRAGHT
GRRCESVINGCKGKPCKNGGTCAVASNTARGFICKCPAGFEGATCENDAR
TCGSLRCLNGGTCISGPRSPTCLCLGPFTGPECQFPASSPCLGGNPCYNQ
GTCEPTSESPFYRCLCPAKFNGLLCHILDYSFGGGAGRDIPPPLIEEACE
LPECQEDAGNKVCSLQCNNHACGWDGGDCSLNFNDPWKNCTQSLQCWKYF
SDGHCDSQCNSAGCLFDGFDCQRAEGQCNPLYDQYCKDHFSDGHCDQGCN
SAECEWDGLDCAEHVPERLAAGTLVVVVLMPPEQLRNSSFHFLRELSRVL
HTNVVFKRDAHGQQMIFPYYGREEELRKHPIKRAAEGWAAPDALLGQVKA
SLLPGGSEGGRRRRELDPMDVRGSIVYLEIDNRQCVQASSQCFQSATDVA
AFLGALASLGSLNIPYKIEAVQSETVEPPPPAQLHFMYVAAAAFVLLFFV
GCGVLLSRKRRRQHGQLWFPEGFKVSEASKKKRREPLGEDSVGLKPLKNA
SDGALMDDNQNEWGDEDLETKKFRFEEPVVLPDLDDQTDHRQWTQQHLDA
ADLRMSAMAPTPPQGEVDADCMDVNVRGPDGFTPLMIASCSGGGLETGNS
EEEEDAPAVISDFIYQGASLHNQTDRTGETALHLAARYSRSDAAKRLLEA
SADANIQDNMGRTPLHAAVSADAQGVFQILIRNRATDLDARMHDGTTPLI
LAARLAVEGMLEDLINSHADVNAVDDLGKSALHWAAAVNNVDAAVVLLKN
GANKDMQNNREETPLFLAAREGSYETAKVLLDHFANRDITDHMDRLPRDI
AQERMHHDIVRLLDEYNLVRSPQLHGAPLGGTPTLSPPLCSPNGYLGSLK
PGVQGKKVRKPSSKGLACGSKEAKDLKARRKKSQDGKGCLLDSSGMLSPV
DSLESPHGYLSDVASPPLLPSPFQQSPSVPLNHLPGMPDTHLGIGHLNVA
AKPEMAALGGGGRLAFETGPPRLSHLPVASGTSTVLGSSSGGALNFTVGG
STSLNGQCEWLSRLQSGMVPNQYNPLRGSVAPGPLSTQAPSLQHGMVGPL
HSSLAASALSQMMSYQGLPSTRLATQPHLVQTQQVQPQNLQMQQQNLQPA
NIQQQQSLQPPPPPPQPHLGVSSAASGHLGRSFLSGEPSQADVQPLGPSS
LAVHTILPQESPALPTSLPSSLVPPVTAAQFLTPPSQHSYSSPVDNTPSH
QLQVPEHPFLTPSPESPDQWSSSSPHSNVSDWSEGVSSPPTSMQSQIARI
PEAFK

1. A compound of Formula I, Formula II, or Formula III: 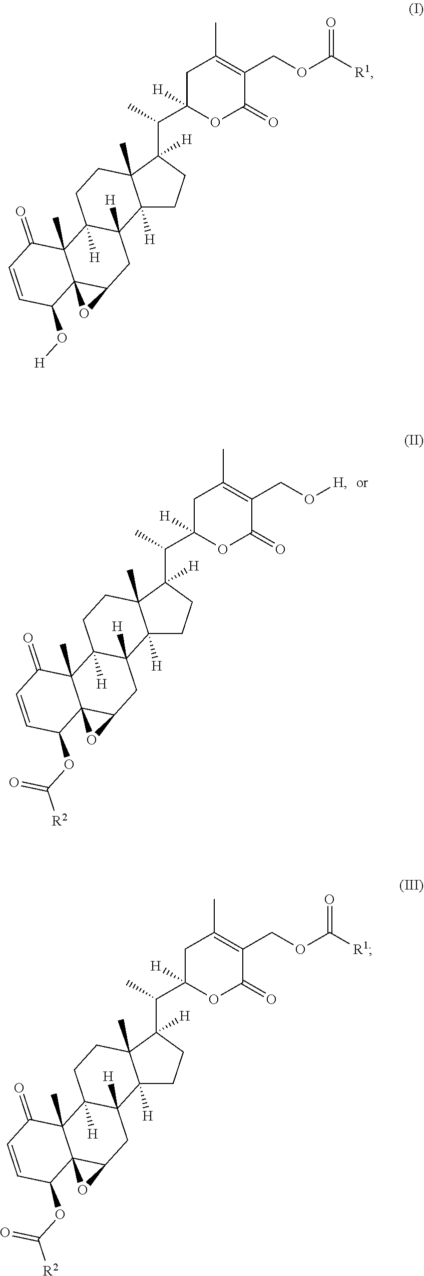
or a pharmaceutically acceptable salt thereof;
wherein:
R¹ and R² are independently selected from C₁-C₆ haloalkyl, N(R³)(R⁴), 3- to 6-membered monocyclic heterocyclyl, and 5- to 10-membered monocyclic or bicyclic heteroaryl, each of which may be optionally substituted with one or more R⁵ groups;
R³ and R⁴ are independently selected at each occurrence from hydrogen, C₁-C₆ alkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, each of which may be substituted with one or more R⁵ groups;
or R³ and R⁴ are brought together with the nitrogen to which they are attached to form a 3- to 6-membered monocyclic heterocycle ring optionally substituted with one or more R⁵ groups;
R⁵ is independently selected at each occurrence from halo, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—;
Rₓ and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more (for example, 1, 2, 3, or 4) Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more (for example 1, 2, 3, or 4) Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

2. (canceled)

3. The compound of claim 1, wherein R¹ is selected from C₁-C₃ fluoroalkyl and C₁-C₃ chloroalkyl.

4. The compound of claim 1, wherein R¹ is dichloromethyl.

5. (canceled)

6. (canceled)

7. (canceled)

8. The compound of claim 1, wherein R¹ is 5- to 10-membered monocyclic or bicyclic heteroaryl optionally substituted with 1, 2, 3, or 4 R⁵ groups.

9. The compound of claim 1, wherein R¹ is selected from pyrrolyl, furanyl, thienyl, pyridyl, benzofuranyl, or quinolinyl optionally substituted with 1, 2, 3, or 4 R⁵ groups.

10. The compound of claim 1, wherein R¹ and/or R² is 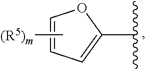
wherein m is 0, 1, 2, or 3.

11. The compound of claim 1, wherein R¹ and/or R² is 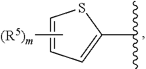
wherein m is 0, 1, 2, or 3.

12. The compound of claim 1, wherein R¹ and/or R² is 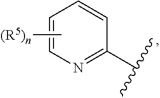
wherein n is 0, 1, 2, 3, or 4.

13. The compound of claim 1, wherein R¹ and/or R² is 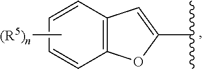
wherein n is 0, 1, 2, 3, or 4.

14. The compound of claim 1, wherein R¹ and/or R² is 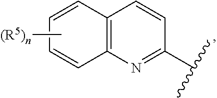
wherein n is 0, 1, 2, 3, or 4.

15. (canceled)

16. The compound of claim 1, wherein R² is selected from C₁-C₃ fluoroalkyl and C₁-C₃ chloroalkyl.

17. The compound of claim 1, wherein R⁸ is dichloromethyl.

18. (canceled)

19. (canceled)

20. (canceled)

21. The compound of claim 1, wherein R² is 5- to 10-membered monocyclic or bicyclic heteroaryl optionally substituted with 1, 2, 3, or 4 R⁵ groups.

22. The compound of claim 1, wherein R² is selected from pyrrolyl, furanyl, thienyl, pyridyl, benzofuranyl, or quinolinyl optionally substituted with 1, 2, 3, or 4 R⁵ groups

23. (canceled)

24. (canceled)

25. (canceled)

26. (canceled)

27. (canceled)

28. The compound of claim 1 selected from: 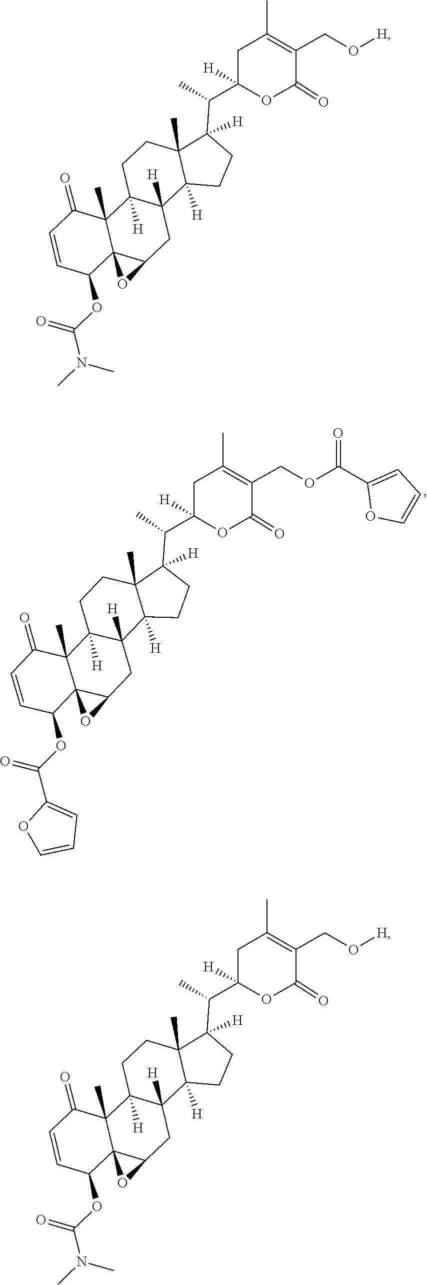 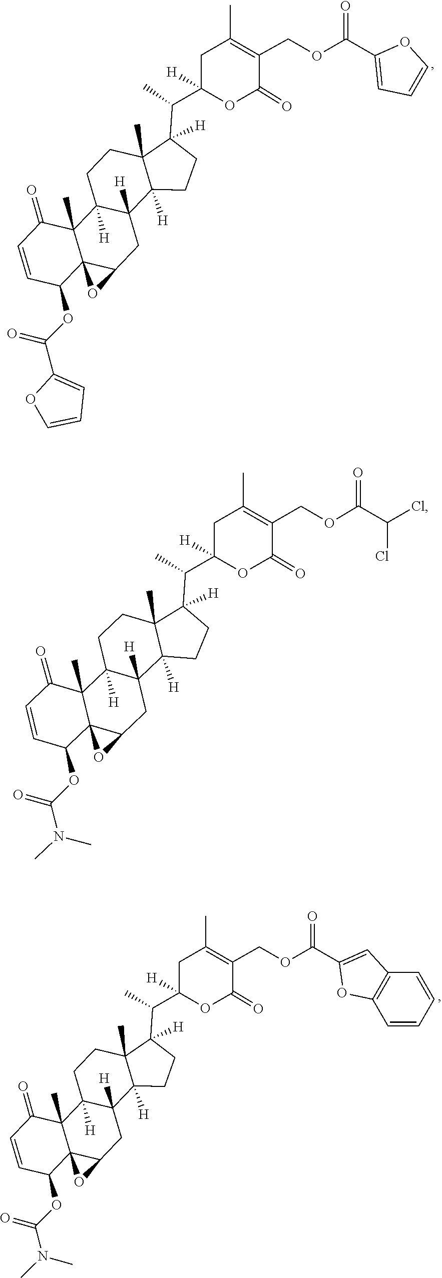 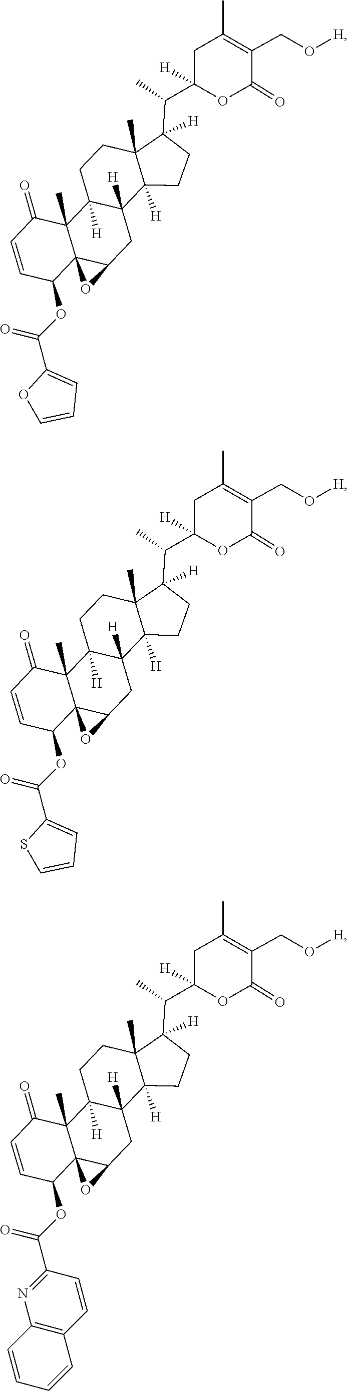 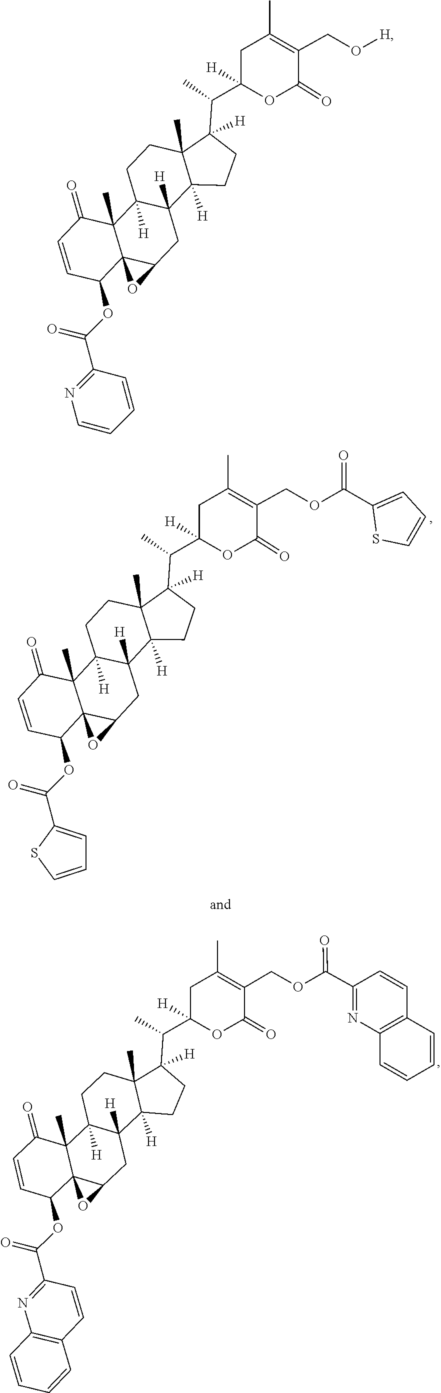
or a pharmaceutically acceptable salt thereof.

29. A pharmaceutical composition comprising the compound of claim 1 and a pharmaceutically acceptable carrier or excipient.

30. A method of treating a cancer, treating an infectious disease, treating a neurological disorder, or reduce Notch1 signaling in a subject, comprising administering to the subject a therapeutically effective amount of the compound of claim 1.

31-40. (canceled)

41. A method of reducing Notch1 signaling in a cell with increased levels of Notch1 signaling comprising contacting the cell with a therapeutically effective amount of the compound of claim 1.

42. (canceled)
